(19) United States
(12) Patent Application Publication
(10) Pub. No.: US 2019/0309264 A1
(43) Pub. Date: Oct. 10, 2019

(54) Three Dimensional Bioprinted Tumor Models for Drug Testing

(71) Applicants: Organovo, Inc., San Diego, CA (US); Oregon Health & Science University, Portland, OR (US)

(72) Inventors: Sharon C. PRESNELL, San Diego, CA (US); Shelby Marie KING, San Diego, CA (US); Deborah Lynn Greene NGUYEN, San Diego, CA (US); Minji JO, San Diego, CA (US); Rosalie Sears PELZ, Portland, OR (US); Brittany ALLEN-PETERSEN, Portland, OR (US); Ellen LANGER, Portland, OR (US)

(21) Appl. No.: 16/325,523

(22) PCT Filed: Aug. 15, 2017
(86) PCT No.: PCT/US2017/046983

§ 371 Date: Feb. 14, 2019

Related U.S. Application Data
(60) Provisional application No. 62/375,016, filed on Aug. 15, 2016.
(60) Provisional application No. 62/430,015, filed on Dec. 5, 2016.

(51) Int. Cl. C12N 5/071; C12N 5/00; C12N 5/077; C12N 5/09; A01K 67/027; G01N 33/50; A61K 49/00; B33Y 70/00; B33Y 80/00

(52) U.S. Cl. CPC C12N 5/0697; A01K 2207/30; C12N 5/0656; C12N 5/0693; A01K 67/0271; G01N 33/5011; A61K 49/0008; B33Y 70/00; B33Y 80/00; A01K 2207/12; C12N 2513/00; A01K 2207/15; A01K 2227/105; A01K 2267/0331; C12N 2503/04; C12N 2503/02; C12N 5/0062

(57) ABSTRACT

Described are three-dimensional, engineered, biological cancer models, methods of producing the same, and methods of identifying a therapeutic agent for cancer in an individual utilizing the three-dimensional, engineered, biological cancer models.

### STATEMENT REGARDING FEDERALLY-SPONSORED RESEARCH AND DEVELOPMENT

This work was performed as part of an SBIR Phase I Contract funded by the NIH/National Cancer Institute (Contract HHSN261201400024C). The U.S. government has certain rights to the invention.

### BACKGROUND OF THE INVENTION

The interaction between cancer cells and the surrounding stromal cells, comprised of fibroblasts, endothelial cells, adipocytes, and immune cells, plays a critical role in cancer initiation, progression, and metastasis. The stromal cells play a structural support role for the epithelium-derived cancer cells, modulate cell signaling and influence angiogenesis and metastasis to distant target tissues.

Dissociation of human tumor samples causes fundamental alterations in the cancer cells as they are removed from their normal three-dimensional environment and results in a loss of heterogeneity that is required to accurately mimic the human disease. Researchers originally addressed the problem by using animal models where small pieces of a human tumor are implanted into immunocompromised mice. Following tumor engraftment, the animals could be used to determine the efficacy of individual chemotherapeutic drugs, or could be used to serially expand the human tumor samples to provide larger amounts of patient tumor material for in vitro or in vivo drug screening.

A significant drawback to a tumor xenograft mouse models is that it is based on an animal that, in comparison to humans, is significantly smaller, has a much higher metabolic rate, is inbred, and has a short life span. Another important difference is the tumor stroma. Because the tumor stroma will be of murine origin, all of its constituent cells will be murine. These stromal cell types include endothelial cells, pericytes, fibroblasts, tumor-associated macrophages, and myeloid-derived suppressor cells. The stromal cells are not merely a scaffold on which tumor cells grow. Rather, they participate actively in tumor formation, progression, and metastasis; produce numerous unique combinations of cytokines/chemokines; and manufacture an extracellular matrix with a variety of adhesion molecules. The presence of the animal stromal cells in the transplanted tumor negatively impacts measurement of biological activities and/or the efficacy of therapeutics being tested, particularly for molecularly targeted therapeutics that disrupt ligand/receptor interactions that rely on species specificity. Additionally, the use of immunocompromised animals prevents the ability to test immunomodulatory therapies.

### BRIEF SUMMARY OF THE INVENTION

The invention provides a three-dimensional, engineered, biological cancer model that is useful in a high-throughput three-dimensional ex vivo system and method for measuring cellular engraftment, remodeling, and proliferation of human tissue in a human bioprinted stromal microenvironment. This system measures the engraftment potential of tumor tissue, bioprinted or normal tissue in a human stromal microenvironment or in the presence of candidate therapies (e.g., chemotherapeutics, immunotherapies, radiotherapies, and cryotherapies). In one embodiment, the ex vivo system described herein advantageously allows more than a 28-day analysis window for measuring the proliferation and engraftment of target tissue. The described human system may be used as an alternative to animal-based xenografts currently in use. And, the system and method provides a more accurate screening of candidate therapies including individual targeted cancer therapeutics.

In one embodiment, provided is a three-dimensional, engineered tissue construct consisting of connective tissue cells derived from the mesoderm and exhibiting a capsule of fibroblasts or fibroblast-like cells on the outer surface of the tissue construct. In one embodiment, the three-dimensional, engineered tissue construct does not comprise a mature perfusable vascular network, does not comprise mature red blood cells, does not comprise innervation, does not comprise neural tissue, or combinations thereof, e.g., at the time of manufacture, use or implantation. In one embodiment, the connective tissue cells are stromal cells. In one embodiment, the stromal cells are breast stromal cells, lung stromal cells, liver stromal cells, kidney stromal cells, prostate stromal cells, intestinal stromal cells, pancreatic stromal cells or skin stromal cells. In one embodiment, the three-dimensional, engineered tissue construct comprises fibroblasts or fibroblast-like cells and at least one other stromal cell type selected from the group consisting of endothelial cells, adipocytes, pre-adipocytes, myoblasts, pericytes, osteocytes, chondrocytes and stellates. In one embodiment, the stromal cells are human mammary fibroblasts, human endothelial cells, human adipocytes, preadipocytes or a mixture of human adipocytes and human preadipocytes.

In one embodiment, the three-dimensional, engineered tissue construct is 1 to 3 mm on each side. In another embodiment, the three-dimensional, engineered tissue construct is 0.25 to 1 mm on each side.

In one embodiment, the capsule of fibroblasts provides a firmness that permits penetration of the construct and deposition of a cellular material within the construct while maintaining the outer form of the construct. In one embodiment, the capsule of fibroblasts provides a firmness that permits incision of the construct and deposition of a cellular material within the construct while maintaining the outer form of the construct. In one embodiment, the capsule of fibroblasts provide a firmness that permits penetration of the construct with a needle and deposition of a cellular material within the construct while maintaining the outer form of the construct.

Also provided is a three-dimensional, engineered tissue construct further comprising at least one type of immune cells. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof. In one embodiment, the immune cells are lymphocytes. In one embodiment, the lymphocytes are selected from the group consisting of natural killer (NK) cells, T cells, B cells and combinations thereof.

Also provided is a method of making the three-dimensional, engineered tissue construct, comprising

(a) preparing a bio-ink comprising the connective tissue cells derived from the mesoderm;

(b) depositing the bio-ink on a biocompatible surface to form an array of cells;

(c) maturing the deposited array of cells in a cell culture media under non-static conditions thereby producing the three-dimensional, engineered, tissue construct with fibroblasts on the outer surface of the construct.

In one embodiment, the bio-ink is deposited by bioprinting. In one embodiment, the bio-ink comprises 55%-75% fibroblasts, 15%-35% endothelial cells, and 0%-20% adipocytes, preadipocytes, or a mixture thereof. In one embodiment, the deposited array of cells is matured in the cell culture medium for 4 to 10 days. In one embodiment, the non-static conditions apply shear stress to the deposited array of cells. In one embodiment, the non-static conditions are created by maturing the deposited array of cells in a rolling bioreactor.

In one embodiment, the bio-ink further comprises at least one type of immune cells. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes, and combinations thereof.

In one embodiment, the immune cells are lymphocytes. In one embodiment, the lymphocytes are selected from the group consisting of natural killer (NK) cells, T cells, B cells and combinations thereof.

Also provided is a three-dimensional, engineered, biological cancer model comprising

(a) a three-dimensional, engineered tissue construct comprising connective tissue cells derived from the mesoderm and exhibiting a capsule of fibroblasts or fibroblast-like cells on the outer surface of the tissue construct, and

(b) an undissociated, primary tumor, primary tumor fragment(s), primary tumor cells or immortalized cells inside the three-dimensional, engineered tissue construct of (a).

In one embodiment, the (a) three-dimensional, engineered tissue construct consists of connective tissue cells derived from the mesoderm and exhibiting a capsule of fibroblasts or fibroblast-like cells on the outer surface of the tissue construct

Also provided is a three-dimensional, engineered, biological cancer model comprising

(a) a three-dimensional, engineered tissue construct comprising connective tissue cells derived from the mesoderm and optionally exhibiting a capsule of fibroblasts or fibroblast-like cells on the outer surface of the tissue construct, and

(b) an undissociated intestinal, lung, gastric, prostate, kidney, skin, ovarian, cervical, uterine, liver, bladder, esophageal, pancreatic or testicular primary tumor(s), primary tumor fragment(s), primary tumor cells or immortalized cells inside the three-dimensional, engineered tissue construct of (a).

In one embodiment, the three-dimensional, engineered tissue construct does not comprise a mature perfusable vascular network, does not comprise mature red blood cells, does not comprise innervation, does not comprise neural tissue, or combinations thereof, e.g., at the time of manufacture, use or implantation.

In one embodiment, the tumor, tumor fragment(s), tumor cells or immortalize cells are breast, lung, liver, kidney, prostate, intestinal, pancreatic or skin tumors, tumor fragment(s), tumor cells or immortalized cells. In one embodiment, the connective tissue cells are stromal cells. In one embodiment, the stromal cells are fibroblasts, endothelial cells, adipocytes, preadipocytes, a mixture of adipocytes and preadipocytes, myoblasts, pericytes, osteocytes, chondrocytes and stellates. In one embodiment, the stromal cells are human mammary fibroblasts, human endothelial cells, human adipocytes, human preadipocytes, or a mixture of human adipocytes and human preadipocytes.

In one embodiment, the three-dimensional, engineered biological cancer model is 1 to 3 mm on each side. In another embodiment, the three-dimensional, engineered biological cancer model is 0.25 to 1 mm on each side.

In one embodiment, a plurality of the cancer models are in the wells of a multi-well plate.

In one embodiment, the three-dimensional, engineered biological cancer model further comprises at least one type of immune cells. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof immune cells are lymphocytes. In one embodiment, the lymphocytes are selected from the group consisting of natural killer (NK) cells, T cells, B cells and combinations thereof.

The invention also provides a three-dimensional, engineered biological cancer model comprising a plurality of (i) undissociated, primary tumors, primary tumor fragments, primary tumor cells or immortalized cells or (ii) a plurality of undissociated intestinal, lung, gastric, prostate, kidney, skin, ovarian, cervical, uterine, liver, bladder, esophageal, pancreatic or testicular primary tumors, primary tumor fragments, primary tumor cells or immortalized cells within the three dimensional, engineered tissue construct comprising connective tissue from the mesoderm. In one embodiment, the plurality of (i) or (ii) are present in separate compartments within the three-dimensional, engineered tissue construct of (a).

In one embodiment, each of the plurality of (i) undissociated, primary tumors, primary tumor fragments, primary tumor cells or immortalized cells or (ii) a plurality of undissociated intestinal, lung, gastric, prostate, kidney, skin, ovarian, cervical, uterine, liver, bladder, esophageal, pancreatic or testicular primary tumors, primary tumor fragments, primary tumor cells or immortalized cells represents a subtype of one or more types of cancer.

Also provided is a three-dimensional, engineered biological cancer model disposed on a solid support. In one embodiment, the model is disposed on a biocompatible membrane that is disposed on the solid support. In one embodiment, the solid support is a multi-well plate.

Also provided is a non-human animal model of cancer comprising a non-human animal implanted therein the three-dimensional, engineered, biological cancer model described herein. In one embodiment, the non-human animal is an immunodeficient rodent.

Also provided is a plurality of the three-dimensional, engineered, biological cancer models in the form of an array. In one embodiment, the array is disposed on a solid support. In one embodiment, the array is disposed on a biocompatible membrane that is disposed on a solid support. In one embodiment, the solid support is a multi-well plate. In one embodiment, each cancer model represents a subtype of one or more types of cancer.

In one embodiment, the plurality of the three-dimensional, engineered, biological cancer models comprises breast cancer tumor(s), tumor fragment(s), tumor cells or immortalized cells. In one embodiment, the array comprises at least two breast cancer models selected from the group consisting of breast cancer subtypes luminal A, luminal B, HER2-enriched (HER2E), basal-like, and normal breast-like. In one embodiment, the array comprises at least two breast cancer models expressing markers selected from the group consisting of ER+, ER−, PR+, PR−, HER2+, HER2−, and ER−/PR−/HER2−.

In one embodiment, the plurality of the three-dimensional, engineered, biological cancer models comprises intestinal cancer tumor(s), tumor fragment(s), tumor cells or immortalized cells. In one embodiment, the array comprises at least two colorectal cancer models selected from the group consisting of colorectal subtypes CMS1, CMS2, CMS3, and CMS4. In one embodiment, the array comprises at least two colorectal models expressing markers selected from the group consisting of MLH1, MLH2, MSH3, MSH6, PMS2, POLE and POLD1.

In one embodiment, the plurality of the three-dimensional, engineered, biological cancer models comprises lung cancer tumor(s), tumor fragment(s), tumor cells or immortalized cells. In one embodiment, the array comprises at least two lung cancer models selected from the group consisting of lung cancer subtypes squamous cell carcinoma, adenocarcinoma, large cell carcinoma, small cell lung carcinoma, and lung carcinoid tumor. In one embodiment, the array comprises at least two lung cancer models expressing markers selected from the group consisting of iNTR, TUBB3, RRM1, ECC1, BRCA1, p53, BCL-2, ALK, MRP2, MSH2, TS, mucin, BAG-1, pERK1/2, pAkt-1, p2′7, PARP-1, ATM and TopIIA.

In one embodiment, the plurality of the three-dimensional, engineered, biological cancer models comprises gastric cancer tumor(s), tumor fragment(s), tumor cells or immortalized cells. In one embodiment, the array comprises at least two gastric cancer models selected from the group consisting of gastric cancer subtypes mesenchymal-like type, microsatellite-unstable type, tumor protein 53 (TP53)-active type and TP53-inactive type. In one embodiment, the array comprises at least two gastric cancer models expressing markers selected from the group consisting of the micro RNAs miR-1, miR-20a, miR-27a, miR-34, miR-196a, miR-378, miR-221, miR376c, miR-423-5p, let-7a, miR-17-5p, miR-21, miR-106a/b, miR-199a-3p, miR-218, miR-223, miR-370, miR-451, miR-486, miR-21, miR-106a, miR-129, and miR-421; TP53; the PTKs TIE-1 and MKK4; FYN; PLK1; GISP/RegIV; EGFR; ERBB2; VEGF; TGF; c-MET; IL-6; IL-11; Cyclin E; Bc1-2; Fas; surviving; Runx3; E-cadherin; WNTSA; IL-1; IL-10; carcinoembryonic antigen (CEA); alpha-fetoprotein (AFP); CA 19-9; CA 72-4; free beta-subunit of human choriogonadotropin (B-HCG), and pepsinogen I/II.

In one embodiment, the plurality of the three-dimensional, engineered, biological cancer models comprises prostate cancer tumor(s), tumor fragment(s), tumor cells or immortalized cells. In one embodiment, the array comprises at least two prostate cancer models selected from the group consisting of prostate cancer subtypes expressing gene fusions ERG, ETV1, ETV4 and FLI1 or selected from the group consisting of prostate cancer subtypes expressing mutations SPOP, FOXA1 and IDH1. In one embodiment, the array comprises at least two prostate cancer models expressing markers selected from the group consisting of NKX3.1, MYC, TMPRSS2-ERG translocations, PTEN, Akt/mTOR, Erk (p42/44), Her2/Neu or SRC tyrosine kinases, WNT, APC, k-RAS, β-catenin, FGFR1, FGF10, EZH2, PCA3, and AR.

In one embodiment, the plurality of the three-dimensional, engineered, biological cancer models comprises kidney cancer tumor(s), tumor fragment(s), tumor cells or immortalized cells. In one embodiment, the array comprises at least two kidney cancer models selected from the group consisting of kidney cancer subtypes renal cell carcinoma and transitional cell carcinoma. In one embodiment, the renal cell carcinoma is selected from the group consisting of clear cell (conventional) (RCC), papillary RCC, chromophobe RCC, renal oncocytoma RCC, unclassified RCC, collecting duct carcinoma, medullary RCC and carcomatoid DCC. In one embodiment, the array comprises at least two kidney cancer models expressing markers selected from the group consisting of neuron-specific enolase (NSE), TRAF-1, Hsp27, IL-1, IL-6, TNF-α, serum amyloid A (SAA), C-reactive protein (CRP), gamma-glutamyl transferase (GGT), tumor necrosis factor-related apoptosis-inducing ligand (TRAIL), cytokeratins (CK), serum M65 (the intact form of cytokeratin 18), hypoxia-inducible transcriptional factors (HIF-1α and HIF-1β), VEGF, Von Hippel-Lindau (VHL), prolyl hydroxylase-3 (PHD3), pyruvate kinase isoenzyme type M2 (TuM2-PK), thymidine kinase 1 (TK1), 20S proteasome, Fetuin A, Osteopontin (OPN), Osteoprotegerin, NMP-22, NGAL, KIM-1, MMPs, and PLIN2.

In one embodiment, the plurality of the three-dimensional, engineered, biological cancer models comprises skin cancer tumor(s), tumor fragment(s), tumor cells or immortalized cells. In one embodiment, the array comprises at least two skin cancer models selected from the group consisting of skin cancer subtypes actinic keratosis, basal cell carcinoma, melanoma, Karposi sarcoma, merkel cell carcinoma, and squamous cell carcinoma. In one embodiment, the melanoma is selected from the group consisting of mutant BRAF, mutant RAS, mutant NF1, and triple-wild type. In one embodiment, the array comprises at least two skin cancer models expressing markers selected from the group consisting of mutant BRAF, mutant RAS, mutant NF1, Triple-WT (wild type), BRAF, NRAS, CDKN2A/B, TP53, PTEN, RAC1, MAP2K1, PPP6C, ARID2, F1, IDH1, RB1, DDX3X, RAC1, IDH1, MRPS31, RPS27, TERT, phospho-MAP2K1/MAP2K2 (MEK1/2), MAPK1/MAPK3 (ERK1/2), CDK4, and CCND1.

In one embodiment, the plurality of the three-dimensional, engineered, biological cancer models comprises ovarian cancer tumor(s), tumor fragment(s), tumor cells or immortalized cells. In one embodiment, the array comprises at least two ovarian cancer models selected from the group consisting of ovarian subtypes serous, endometrioid, clear cell and mucinous. In one embodiment, the array comprises at least two ovarian cancer models expressing markers selected from the group consisting of B-RAF, K-RAS, TP53, BRCA1/2, CA125, CA 19.9, CA 15.3, TAG.72, MSH2, MLH1, MLH6, PMS1, PMS2, ESR2, BRIP1, MSH6, RAD51C, RAD51D, CDH1, CHEK2, PALB2, RAD50, OVX1, sFas, CYFRA 21.1, VEGF, human kallikrein 10 (hK10), Alpha-fetoprotein (αFP), M-CSF, and LDH, inhibin α, betaA, and betaB subunits.

In one embodiment, the plurality of the three-dimensional, engineered, biological cancer models comprises cervical cancer tumor(s), tumor fragment(s), tumor cells or immortalized cells. In one embodiment, the array comprises at least two cervical cancer models selected from the group consisting of cervical cancer subtypes squamous cell carcinoma and adenocarcinoma. In one embodiment, the array comprises at least two cervical cancer models expressing markers selected from the group consisting of p16ink4a, MCM 3 and 5, CDC6, Geminin, Cyclins A-D, TOPO2A, CDCA1, BIRC5, UBE2C, CCNB1, CCNB2, PLOD2, NUP210, MELK, CDC20, IL8, INDO, ISG15, ISG20, AGRN, DTXL, MMP1, MMP3, CCL18, STAT1, ribosomal protein S12, the mitochondrial subunit NADH dehydrogenase 4, 16S ribosomal RNA (rRNA), and capping protein muscle Z-line al.

In one embodiment, the plurality of the three-dimensional, engineered, biological cancer models comprises uterine cancer tumor(s), tumor fragment(s), tumor cells or immortalized cells. In one embodiment, the array comprises at least two uterine cancer models selected from the group consisting of uterine cancer subtypes endometrioid, adenocarcinoma, serous adenocarcinoma, adenosquamous carcinoma and carcinomasarcoma. In one embodiment, the array comprises at least two uterine cancer models expressing markers selected from the group consisting of MLH1, MSH2, MSH6, PMS2, EPCAM, PTEN, BRCA1, BRCA2, TP53, MUTYH, CDKN2A, PGR, and CHEK2.

In one embodiment, the plurality of the three-dimensional, engineered, biological cancer models comprises liver cancer tumor(s), tumor fragment(s), tumor cells or immortalized cells. In one embodiment, the array comprises at least two liver cancer models selected from the group consisting of liver cancer subtypes hepatocellular carcinoma (HCC), cholangiocarcinoma, angiosarcoma, and hepatoblastoma. In one embodiment, the array comprises at least two liver cancer models expressing markers selected from the group consisting of AFP-L1, AFP-L2, AFP-L3, HSP70, HSP27, Glypican-3 (GPC3), squamous cell carcinoma antigen (SCCA), Golgi protein 73 (GP73, also known as Golph2 and GOLM1), Tumor-associated glycoprotein 72 (TAG-72), Zinc-α2-glycoprotein (ZAG), Des-γ-carboxyprothrombin (DCP), γ-glutamyl transferase (GGT), α-1-fucosidase (AFU), Transforming growth factor-β1 (TGF-β1), VEGF, microRNAs such as miR-500, miR-122, miR-29, and miR-21; Δ-like 1 homolog (DLK1), Villin1 (Vil1), TP53, CD34, RGS5, THY1, ADAMTS1, MMP2, MMP14, keratin 17, keratin 19, and mucin 1.

In one embodiment, the plurality of the three-dimensional, engineered, biological cancer models comprises bladder cancer tumor(s), tumor fragment(s), tumor cells or immortalized cells. In one embodiment, the array comprises at least two bladder cancer models selected from the group consisting of bladder cancer subtypes urothelial carcinoma, squamous cell carcinoma, adenocarcinoma, sarcoma and small cell anaplastic cancer. In one embodiment, the array comprises at least two bladder cancer models expressing markers selected from the group consisting of HRAS, NRAS, KRAS2, FGFR3, ERBB2, CCND1, MDM2, E2F3, RASSF1A, FHIT, CDKN2A, PTCH, DBC1, TSC1, PTEN, RB1, TP53, SULF1, the lysosomal cysteine proteinases cathepsins B, K, and L; RGS1, RGS2, THBS1, THBS2, VEGFC, NRP2, CTSE, MMP2, CCNA2, CDC2, CDC6, TOP2A, SKALP PRKAG1, GAMT, ACOX1, ASAH1, SCD, AF1Q, AREG, DUSP6, LYAR, MAL, and RARRES

In one embodiment, the plurality of the three-dimensional, engineered, biological cancer models comprises esophageal cancer tumor(s), tumor fragment(s), tumor cells or immortalized cells. In one embodiment, the array comprises at least two esophageal cancer models selected from the group consisting of esophageal cancer subtypes squamous-cell carcinoma and adenocarcinoma. In one embodiment, the array comprises at least two esophageal cancer models expressing markers selected from the group consisting of SMYD3, RUNX1, CTNNA3, RBFOX1, CDKN2A/2B, CDK14, ERBB2, EGFR, RB1, GATA4/6, CCND1, MDM2, TP53, ARID1A, and SMARCA4.

In one embodiment, the plurality of the three-dimensional, engineered, biological cancer models comprises pancreatic cancer tumor(s), tumor fragment(s), tumor cells or immortalized cells. In one embodiment, the array comprises at least two pancreatic cancer models selected from the group consisting of pancreatic cancer subtypes exocrine and pancreatic neuroendocrine tumors (PNETs). In one embodiment, the array comprises at least two pancreatic cancer models selected from the group consisting of pancreatic cancer subtypes squamous, pancreatic progenitor, immunogenic and aberrantly differentiated endocrine exocrine (ADEX). In one embodiment, the array comprises at least two pancreatic cancer models expressing markers selected from the group consisting of TP53, KDM6A, MLL2, MLL3, PDX1, MNX1, GATA6, HNF1B, transcription factors PDX1, MNX1, HNF4G, HNF4A, HNF1B, HNF1A, FOXA2, FOXA3, HES1, NR5A2, MIST1 (also known as BHLHA15A), and RBPJL; INS, NEUROD1, NKX2-2, MAFA, AMY2B, PRSS1, PRSS3, CEL, and INS.

In one embodiment, the plurality of the three-dimensional, engineered, biological cancer models comprises testicular cancer tumor(s), tumor fragment(s), tumor cells or immortalized cells. In one embodiment, the array comprises at least two testicular cancer models selected from the group consisting of testicular cancer subtypes germ cell and stromal tumors. In one embodiment, the array comprises at least two testicular cancer models expressing markers selected from the group consisting of AFP, HCG, LDH, HMGA1, HMGA2, OCT3/4 (a transcription factor of the family of octamer-binding proteins (also known as the POU homeodomain proteins)), SOX2, SOX17, CDK10 and genetic loci located within KITLG, TERT, SPRY4, BAK1, DMRT1, ATF7IP, HPGDS, SMARCAD1, SEPT4, TEX14, RAD51C, PPM1E, TRIM37, MAD1L1, TEX14, SKA2, SMARCAD1, RFWD3, and RAD51C.

In one embodiment, the plurality of the three-dimensional, engineered, biological cancer models further comprises at least one type of immune cells in culture media that is in contact with and/or within the cancer models. In one embodiment, the immune cells are myeloid-lineage cells. In one embodiment, the myeloid cells are selected from the group consisting of monocytes, macrophages, pre-differentiated macrophages, neutrophils, basophils, eosinophils, dendritic cells, megakaryocytes, and combinations thereof. In one embodiment, the immune cells are lymphocytes. In one embodiment, the lymphocytes are selected from the group consisting of natural killer (NK) cells, T cells, B cells and combinations thereof.

In one embodiment, the plurality of the three-dimensional, engineered, biological cancer models are in culture media under non-static culture conditions. In one embodiment, the non-static culture conditions is lateral flow across the cancer models. In one embodiment, the cancer models are in culture media under static culture conditions.

Also provided is the plurality of the three-dimensional, engineered, biological cancer models for use in a high throughput assay.

Also provided is a method of making the three-dimensional, engineered biological cancer model, comprising

(a) creating an opening in the three-dimensional, engineered tissue construct,

(b) inserting an undissociated, primary tumor, primary tumor fragment(s), primary tumor cells or immortalized cells into the opening, and

(c) maturing the three-dimensional, engineered biological cancer model in cell culture media to allow the opening to close.

Also provided is a method of identifying a therapeutic agent for the treatment of cancer, comprising

(a) contacting a candidate therapeutic agent with the three-dimensional, engineered biological cancer model or the plurality of the three-dimensional, engineered, biological cancer models;

(b) measuring an effect on the primary tumor, primary tumor fragment(s), primary tumor cells or immortalized cells and/or the connective tissue cells derived from the mesoderm; and

(c) selecting the therapeutic agent for treatment of cancer based upon the measured effect.

In one embodiment, the method is for identifying a therapeutic agent for treatment of cancer in an individual and the tumor, tumor fragment(s), tumor cells or immortalized cells derived are from that individual.

In one embodiment, the cancer is breast cancer, lung cancer, liver cancer, kidney cancer, prostate cancer, intestinal cancer, pancreatic cancer or skin cancer. In one embodiment, the cancer is gastric cancer, ovarian cancer, cervical cancer, uterine cancer, bladder cancer, esophageal cancer, or testicular cancer.

In one embodiment, the stromal cells are breast stromal cells, and the tumor, tumor fragment(s), tumor cells or immortalized cells are derived from an individual having breast cancer.

In one embodiment, the stromal cells are lung stromal cells, and the tumor, tumor fragment(s), tumor cells or immortalized cells are derived from an individual having lung cancer.

In one embodiment, the stromal cells are liver stromal cells, and the tumor, tumor fragment(s), tumor cells or immortalized cells are derived from an individual having liver cancer.

In one embodiment, the stromal cells are kidney stromal cells, and the tumor, tumor fragment(s), tumor cells or immortalized cells are derived from an individual having kidney cancer.

In one embodiment, the stromal cells are prostate stromal cells, and the tumor, tumor fragment(s), tumor cells or immortalized cells are derived from an individual having prostate cancer.

In one embodiment, the stromal cells are intestinal stromal cells, and the tumor, tumor fragment(s), tumor cells or immortalized cells are derived from an individual having intestinal cancer.

In one embodiment, the stromal cells are pancreatic cells, and the tumor, tumor fragment(s), tumor cells or immortalized cells are derived from an individual having pancreatic cancer.

In one embodiment, the stromal cells are skin cells, and the tumor, tumor fragment(s), tumor cells or immortalized cells are derived from an individual having skin cancer.

In one embodiment, the stromal cells are gastric stromal cells, and the tumor, tumor fragment(s), tumor cells or immortalized cells are derived from an individual having gastric cancer.

In one embodiment, the stromal cells are ovarian stromal cells, and the tumor, tumor fragment(s), tumor cells or immortalized cells are derived from an individual having ovarian cancer.

In one embodiment, the stromal cells are cervical stromal cells, and the tumor, tumor fragment(s), tumor cells or immortalized cells are derived from an individual having cervical cancer.

In one embodiment, the stromal cells are uterine stromal cells, and the tumor, tumor fragment(s), tumor cells or immortalized cells are derived from an individual having uterine cancer.

In one embodiment, the stromal cells are bladder stromal cells, and the tumor, tumor fragment(s), tumor cells or immortalized cells are derived from an individual having bladder cancer.

In one embodiment, the stromal cells are esophageal stromal cells, and the tumor, tumor fragment(s), tumor cells or immortalized cells are derived from an individual having esophageal cancer.

In one embodiment, the stromal cells are testicular stromal cells, and the tumor, tumor fragment(s), tumor cells or immortalized cells are derived from an individual having testicular cancer.

In one embodiment, each cancer model of the plurality of cancer models represent subtypes of a particular type of cancer.

In one embodiment, the cancer models are in culture media under non-static culture conditions. In one embodiment, the non-static conditions is lateral flow across the cancer models. In one embodiment, the cancer models are in culture media under static culture conditions. In one embodiment, the assay is carried out in a high throughput assay format.

In one embodiment, the effect on the primary tumor, primary tumor fragment(s), primary tumor cells or immortalized cells is measured by one or more of

(a) detecting any reduction of the size of the primary tumor or primary tumor fragment(s);

(b) detecting any reduction in the growth of the primary tumor, primary tumor fragment(s), primary tumor cells or immortalized cells;

(c) detecting apoptosis in the primary tumor, primary tumor fragment(s), primary tumor cells or immortalized cells;

(d) detecting the extent of damage of the primary tumor, primary tumor fragment(s), primary tumor cells or immortalized cells;

(e) detecting reduced viability of the primary tumor, primary tumor fragment(s), primary tumor cells or immortalized cells;

detecting the appearance, level or disappearance of cell markers on the primary tumor, primary tumor fragment(s), primary tumor cells or immortalized cells;

(g) detecting a change in the rate of proliferation or growth of the primary tumor, primary tumor fragment(s), primary tumor cells or immortalized cells;

(h) detecting a change in the staining of the primary tumor, primary tumor fragment(s), primary tumor cells or immortalized cells;

detecting a change in RNA or DNA and/or expression thereof in the primary tumor, primary tumor fragment(s), primary tumor cells or immortalized cells;

detecting a change in protein expression in the primary tumor, primary tumor fragment(s), primary tumor cells or immortalized cells;

(k) detecting a change in cytokine expression and/or secretion and/or level in the primary tumor, primary tumor fragment(s), primary tumor cells or immortalized cells; or

(l) detecting T-cell recruitment, myeloid-lineage cell recruitment, infiltration and/or activation in the primary tumor, primary tumor fragment(s), primary tumor cells or immortalized cells.

Also provided is a method of screening a candidate therapeutic agent for treatment of cancer, comprising:

(a) contacting the non-human animal model described herein with the candidate therapeutic agent;

(b) measuring an effect on the primary tumor, primary tumor fragment(s), primary tumor cells or immortalized cells and/or the connective tissue cells derived from the mesoderm; and

(c) selecting the therapeutic agent for treatment of cancer based upon the measured effect.

In some embodiments, the effect on the primary tumor, primary tumor fragment(s), primary tumor cells or immortalized cells is measured by one or more of

(a) detecting any reduction of the size of the primary tumor or primary tumor fragment(s);

(b) detecting any reduction in the growth of the primary tumor, primary tumor fragment(s), primary tumor cells or immortalized cells;

(c) detecting apoptosis in the primary tumor, primary tumor fragment(s), primary tumor cells or immortalized cells;

(d) detecting the extent of damage of the primary tumor, primary tumor fragment(s), primary tumor cells or immortalized cells;

(e) detecting reduced viability of the primary tumor, primary tumor fragment(s), primary tumor cells or immortalized cells;

(f) detecting the appearance, level or disappearance of cell markers on the primary tumor, primary tumor fragment(s), primary tumor cells or immortalized cells;

(g) detecting a change in the rate of proliferation or growth of the primary tumor, primary tumor fragment(s), primary tumor cells or immortalized cells;

(h) detecting a change in the staining of the primary tumor, primary tumor fragment(s), primary tumor cells or immortalized cells;

(i) detecting a change in RNA or DNA and/or expression thereof in the primary tumor, primary tumor fragment(s), primary tumor cells or immortalized cells;

(j) detecting a change in protein expression in the primary tumor, primary tumor fragment(s), primary tumor cells or immortalized cells;

(k) detecting a change in cytokine expression and/or secretion and/or level in the primary tumor, primary tumor fragment(s), primary tumor cells or immortalized cells; or

(l) detecting T-cell recruitment, myeloid-lineage cell recruitment, infiltration and/or activation in the primary tumor, primary tumor fragment(s), primary tumor cells or immortalized cells.

Also provided is a non-human animal model of cancer comprising: (a) a three-dimensional, engineered, biological cancer model comprising a three-dimensional, engineered tissue construct comprising a stromal tissue and a tumor tissue, wherein the tumor tissue is inside the stromal tissue, and the stromal tissue was bioprinted from a stromal bio-ink; and (b) a non-human animal comprising the three-dimensional, engineered, biological cancer model, provided that the cancer model is implanted into the non-human animal after the tumor tissue is cohered to the stromal tissue.

In one embodiment, the non-human animal is a genetically engineered rodent.

In one embodiment, the non-human animal is an immunodeficient rodent.

In one embodiment, the three-dimensional, engineered, biological cancer model does not comprise a mature perfusable vascular network, does not comprise mature red blood cells, does not comprise innervation, does not comprise neural tissue, or combinations thereof.

In one embodiment, the tumor tissue comprises a plurality of undissociated, primary tumor, primary tumor fragments, primary tumor cells or immortalized cells.

In one embodiment, the stromal tissue comprises stromal cells selected from the group consisting of fibroblasts, endothelial cells, adipocytes, pre-adipocytes, a mixture of adipocytes and preadipocytes, myoblasts, pericytes, osteocytes, chondrocytes and stellates.

In one embodiment, the stromal tissue comprises breast stromal cells, lung stromal cells, liver stromal cells, kidney stromal cells, prostate stromal cells, intestinal stromal cells, pancreatic stromal cells or skin stromal cells.

In one embodiment, the tumor tissue comprises a tumor tissue selected from the group consisting of intestinal, lung, gastric, prostate, kidney, skin, ovarian, cervical, uterine, liver, bladder, esophageal, pancreatic and testicular tumor tissue.

In one embodiment, the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231.

In one embodiment, the tumor tissue is a pancreatic tumor tissue, and the pancreatic tumor tissue comprises markers from a pancreatic cell line. In one embodiment, the pancreatic cell line is selected from the group consisting of OPTR3099C, CAPAN1, CAPAN2, PANC1, MIAPACA2, CFPAC1, ASPC1, COL0357, PANC89, or HPAFII.

In one embodiment, the tumor tissue is surrounded on all sides by the stromal tissue.

In one embodiment, the cancer model is substantially free of pre-formed scaffold.

In one embodiment, the tumor tissue was bioprinted.

In one embodiment, the cancer model is about 1 to about 3 mm on each side.

In one embodiment, the cancer model is about 0.25 to about 1 mm on each side.

In one embodiment, the non-human animal model of cancer further comprises at least one type of immune cells. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof. In one embodiment, the immune cells are lymphocytes. In one embodiment, the lymphocytes are selected from the group consisting of natural killer (NK) cells, T cells, B cells and combinations thereof.

In one embodiment, the three-dimensional, engineered, biological cancer model of (a) was subcutaneously implanted into the non-human animal.

In one embodiment, the stromal tissue comprises connective tissue cells derived from a mesoderm.

In one embodiment, the tumor tissue comprises primary cancer cells from a patient tumor.

Also provided is a method of making a non-human animal model of cancer comprising: depositing a stromal bio-ink by bioprinting, wherein the stromal bio-ink comprises a stromal tissue; depositing a tumor tissue inside the stromal tissue; maturing the deposited stromal tissue and the deposited tumor tissue in a cell culture media to allow the stromal tissue to cohere to the tumor tissue to form a three-dimensional, engineered, biological cancer model; and implanting the cohered three-dimensional, engineered, biological cancer model into a non-human animal.

Also provided is a method of identifying a therapeutic agent for cancer comprising: depositing a stromal bio-ink by bioprinting, the stromal bio-ink comprising a stromal tissue; depositing a tumor tissue inside the stromal tissue, wherein the tumor tissue comprises a plurality of cancer cells; maturing the deposited stromal tissue and the deposited tumor tissue in a cell culture media to allow the stromal tissue to cohere to the tumor tissue to form a three-dimensional, engineered, biological cancer model; implanting the cohered three-dimensional, engineered, biological cancer model into a non-human animal; applying a candidate therapeutic agent to the cancer model; measuring viability of the cancer cells; and selecting a therapeutic agent based on the measured viability of the cancer cells.

In one embodiment of any of the above methods, the non-human animal is a genetically engineered rodent. In one embodiment of any of the above methods, the non-human animal is an immunodeficient rodent.

In one embodiment of any of the above methods, the three-dimensional, engineered, biological cancer model does not comprise a mature perfusable vascular network, does not comprise mature red blood cells, does not comprise innervation, does not comprise neural tissue, or combinations thereof.

In one embodiment of any of the above methods, the tumor tissue comprises a plurality of undissociated, primary tumor, primary tumor fragments, primary tumor cells or immortalized cells.

In one embodiment of any of the above methods, the stromal tissue comprises stromal cells selected from the group consisting of fibroblasts, endothelial cells, adipocytes, pre-adipocytes, a mixture of adipocytes and preadipocytes, myoblasts, pericytes, osteocytes, chondrocytes and stellates.

In one embodiment of any of the above methods, the stromal tissue comprises breast stromal cells, lung stromal cells, liver stromal cells, kidney stromal cells, prostate stromal cells, intestinal stromal cells, pancreatic stromal cells or skin stromal cells.

In one embodiment of any of the above methods, the tumor tissue comprises a tumor tissue selected from the group consisting of intestinal, lung, gastric, prostate, kidney, skin, ovarian, cervical, uterine, liver, bladder, esophageal, pancreatic and testicular tumor tissue.

In one embodiment of any of the above methods, the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231.

In one embodiment of any of the above methods, the tumor tissue is a pancreatic tumor tissue, and the pancreatic tumor tissue comprises markers from a pancreatic cell line, such as OPTR3099C, CAPAN1, CAPAN2, PANC1, MIAPACA2, CFPAC1, ASPC1, COLO357, PANC89, or HPAFII.

In one embodiment of any of the above methods, the tumor tissue is surrounded on all sides by the stromal tissue.

In one embodiment of any of the above methods, the cancer model is substantially free of pre-formed scaffold.

In one embodiment, any of the above methods further comprises the step of depositing the tumor tissue by bioprinting.

In one embodiment of any of the above methods, the bioprinting is by extrusion.

In one embodiment of any of the above methods, the cancer model is about 1 to about 3 mm on each side.

In one embodiment of any of the above methods, the cancer model is about 0.25 to about 1 mm on each side.

In one embodiment, any of the above methods further comprises the step of depositing at least one type of immune cells. In one embodiment, the step of depositing the at least one type of immune cells is by bioprinting. In one embodiment, the bioprinting is by extrusion. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof. In one embodiment, the immune cells are lymphocytes. In one embodiment, the lymphocytes are selected from the group consisting of natural killer (NK) cells, T cells, B cells and combinations thereof.

In one embodiment of any of the above methods, the step of implanting the cancer model into the non-human animal is by subcutaneous implantation. In one embodiment, the tumor model is implanted into a flank of the non-human animal.

In one embodiment of any of the above methods, the stromal tissue comprises connective tissue cells derived from a mesoderm.

In one embodiment of any of the above methods, the cancer cells are primary cancer cells from a patient tumor.

In one embodiment of any of the above methods, the candidate therapeutic agent is applied to the implanted cancer model.

In one embodiment, any of the above methods further comprises the step of removing the implanted cancer model from the non-human animal, wherein the candidate therapeutic agent is applied to the cancer model after the cancer model is removed from the non-human animal.

In one embodiment of any of the above methods, the candidate therapeutic agent is an immunotherapy. In one embodiment, the immunotherapy is an adoptive T cell transfer, an immune checkpoint inhibitor to activate Tc and NK cells, or an immune cell reprogramming and depletion.

Also provided is a three-dimensional, engineered, biological breast cancer model comprising: (a) breast stromal tissue, the stromal tissue comprising fibroblasts, endothelial cells, adipocytes, and monocytes; and (b) breast cancer tumor tissue, the tumor tissue comprising breast cancer cells, fibroblasts, endothelial cells, and monocytes; the tumor tissue surrounded on all sides by the stromal tissue to form the three-dimensional, engineered, biological breast cancer model; provided that the stromal tissue was bioprinted from a stromal bio-ink, the tumor tissue was bioprinted from a tumor bio-ink, or both the stromal tissue and the tumor tissue were bioprinted from their respective bio-inks.

In one embodiment, the model is substantially free of pre-formed scaffold.

In one embodiment, the breast cancer cells are derived from a breast cancer cell line. In one embodiment, the breast cancer cell line is selected from the group consisting of ER+, ER−, PR+, PR−, HER2+, HER2−, and ER−/PR−/HER2−.

In one embodiment, the breast cancer cells are primary cancer cells from a patient tumor.

In one embodiment, the breast cancer tumor tissue is completely surrounded on all sides by the breast stromal tissue to form the three-dimensional, engineered, biological breast cancer model.

In one embodiment, the breast cancer model further comprises a plurality of macrophages that were differentiated from the monocytes.

Also provided is a method of fabricating a three-dimensional, engineered, biological breast cancer model comprising: (a) preparing a stromal bio-ink, the stromal bio-ink comprising a plurality of stromal cell types, the stromal cell types comprising: an extrusion compound, fibroblasts, endothelial cells, monocytes, and adipocytes; (b) preparing a tumor bio-ink, the tumor bio-ink comprising: an extrusion compound, a breast cancer cell type, fibroblasts, and monocytes; (c) depositing the stromal bio-ink and the tumor bio-ink such that the tumor bio-ink is embedded in the stromal bio-ink and in contact with the stromal bio-ink on all sides, and (d) maturing the deposited bio-ink in a cell culture media to remove the extrusion compound to allow the cells to cohere to form a three-dimensional, engineered, biological breast cancer model.

In one embodiment, the bio-ink is deposited by bioprinting.

In one embodiment, the breast cancer cell type comprises a breast cancer cell line.

In one embodiment, the breast cancer cell line is selected from the group consisting of ER+, ER−, PR+, PR−, HER2+, HER2−, and ER−/PR−/HER2−.

In one embodiment, the cancer cell type comprises primary breast cancer cells from a patient tumor.

In one embodiment, the method further comprises the step of allowing the monocytes to differentiate into a plurality of macrophages. In one embodiment, the method further comprises the step of allowing the macrophages to migrate towards the breast cancer cell types.

In one embodiment, the method further comprises the steps of applying a candidate therapeutic agent to the three-dimensional, engineered, biological breast cancer model; measuring viability of the cancer cells; and selecting a therapeutic agent for the individual based on the measured viability of the cancer cells. In one embodiment, the candidate therapeutic agent is an immunotherapy. In one embodiment, the immunotherapy is an adoptive T cell transfer, an immune checkpoint inhibitor to activate Tc and NK cells, or an immune cell reprogramming and depletion.

### BRIEF DESCRIPTION OF THE DRAWINGS/FIGURES

FIG. 1 shows bioprinted stromal tissue or stromal box.

FIG. 2 shows the hematoxylin and eosin staining (H&E stain) of primary breast tumor tissue perfused in a rolling reactor for 7 days prior to implantation into bioprinted breast stromal tissue.

FIGS. 3A-3E show the H&E staining of primary breast tumor tissue post implantation into bioprinted breast stromal tissues and perfused in a rolling reactor for 7 days or 28 days.

FIGS. 4A-4C show proliferation of stromal tissue in the presence of implanted primary tumor tissue using proliferating cell nuclear antigen (PCNA) (green in color figures).

FIGS. 5A-5C show organization of endothelial cell networks in the stromal tissue in the presence of implanted primary tumor tissue by staining for CD31 (Abcam Catalog No. 7653300).

FIGS. 6A-6E show the H&E staining of remolded primary breast tumor tissue (increased epithelial cells in starting material) excised and implanted into stromal tissue and perfused in a rolling reactor for 7 days.

FIGS. 7A-7E depict lateral flow of media across a bioprinted breast 3D cancer model comprising MCF7 breast cancer cells surrounded by stroma comprising mammary fibroblasts, endothelial cells, and adipocytes. FIG. 7A is a photograph of 6 bioreactors (from Kiyatec, Inc.) that are perfused in parallel with cell culture medial to provide lateral flow. FIGS. 7B-7E are micrographs showing that flow conditions enhance ECM organization and tissue cohesion.

FIGS. 8A-8B show that 3D bioprinted breast cancer models subject to flow perfusion exhibit increased resistance to doxorubicin, thus providing a more accurate model compared to 2D co-cultured cells and static 3D bioprinted cells. FIG. 8A is a graph showing doxorubicin toxicity to 3D breast cancer models for vehicle (control) and increasing concentrations of doxorubicin. FIG. 8B is a graph showing the doxorubicin LD₅₀ (μM) of cultured 2D normal human mammary fibroblasts (NHMF), 2D human umbilical vein endothelial cells (HUVEC), 2D subcutaneous pre-adipocytes (SPA), 2D MCF7 cancer cells, 2D co-cultured cells (a mixture of all cell types), 3D breast cancer models with static culture and 3D breast cancer models with flow perfusion (the 3D models had the same ratio of cell types as in the 2D co-cultured cells).

FIG. 9 depicts micrographs showing that cell-type specific effects can be observed in 3D breast cancer models subject to flow perfusion. The upper left hand panel is a micrograph showing stromal cells contacted with vehicle with flow perfusion. The upper right hand panel is a micrograph showing the effect of 10 μM doxorubicin on stromal cells subject to flow perfusion. The lower left hand panel is a micrograph showing cancer cells contacted with vehicle with flow perfusion. The lower right hand panel is a micrograph showing the effect of 10 μM doxorubicin on cancer cells subject to flow perfusion.

FIGS. 10A-C shows the H&E staining (FIG. 10A) and immunofluorescence (FIG. 10B) of a 3D bioprinted breast tissue containing breast cancer cells, and the H&E staining of a xenograft derived from a bioprinted breast tissue containing breast cancer cells (FIG. 10C).

FIGS. 11A-B shows the growth of a 3D bioprinted tissue containing pancreatic cancer cells subcutaneously xenografted into three individual immunodeficient mice over time (FIG. 11A) and a representative H&E staining of pancreatic tumor tissue generated from the xenografted, 3D bioprinted pancreatic tissue (FIG. 11B).

FIGS. 12A-B show the H&E staining, of a breast cancer tissue construct comprising immune cells at day 7 post bio-printing. FIGS. 12C-D show the H&E staining of this breast cancer tissue construct comprising immune cells at day 14 post bio-printing.

### DETAILED DESCRIPTION OF THE INVENTION

In one embodiment, the invention provides a three-dimensional, engineered, biological cancer model comprising a plurality of connective tissue cells derived from the mesoderm surrounding on all sides an undissociated, primary, cancer tumor, primary tumor fragment (s) or primary tumor cells. In one embodiment, the plurality connective tissue cells derived from the mesoderm are stromal cells. In another embodiment, the stromal cell types are human mammary fibroblasts, human endothelial cells, and human adipocytes or a mixture of human adipocytes and human preadipocytes. In another embodiment, the cancer tumor or tumor fragment(s) are derived from a different patient than the plurality of stromal cell types. In another embodiment, the three-dimensional, engineered, biological cancer model (a) does not comprise a mature perfusable vascular network, (b) does not comprise mature red blood cells, (c) does not contain innervation, (d) does not contain neural tissue, or combinations of (a)-(d).

In another embodiment, the invention provides bioprinted constructs that may be matured in cell culture media to provide a three-dimensional, engineered, biological tissue model. The bioprinted construct comprises a plurality of connective tissue cells derived from the mesoderm in a number of stacked arrays. In one embodiment, the plurality of connective tissue cells derived from the mesoderm are part of a bio-ink. In another embodiment, the bio-ink further comprises an extrusion compound. In another embodiment, the plurality of connective tissue cells derived from the mesoderm are limited to human mammary fibroblasts, human endothelial cells, and human preadipocytes or a mixture of human preadipocytes and adipocytes. In another embodiment, the plurality of connective tissue cells derived from the mesoderm comprise myofibroblasts.

In another embodiment, the invention provides methods of fabricating a three-dimensional, engineered, biological tissue model, the method comprising: preparing a bio-ink, the bio-ink comprising a plurality of connective tissue cells derived from the mesoderm cell types; depositing the bio-ink on a biocompatible surface to give a plurality of connective tissue cells derived from the mesoderm cell types in a number of stacked arrays; and maturing the plurality of connective tissue cells derived from the mesoderm cell types in the number of stacked arrays in a cell culture media to allow the cells to cohere to form a three-dimensional, engineered, tissue model. In one embodiment the bio-ink further comprises an extrusion compound. In another embodiment, the extrusion compound is removed when the plurality of stromal cell types in the stacked arrays are matured in culture media. In another embodiment, the plurality of connective tissue cells derived from the mesoderm are stromal cells. In another embodiment, the stromal cells in the bio-ink are limited to human mammary fibroblasts, human endothelial cells, and human preadipocytes. In one embodiment, upon maturation in the cell culture media, the preadipocytes mature to form adipocytes as are found in stromal breast tissue.

In another embodiment, the invention provides methods of fabricating a three-dimensional, engineered, biological cancer model, the method comprising: inserting primary cancer tumors cells, an undissociated, primary, patient-derived cancer tumor and/or tumor fragment(s) or immortalized cells into the three-dimensional, engineered, tissue model; and maturing in a cell culture media to give the three-dimensional, engineered, biological cancer model. In one embodiment, the tumor cells, tumor and/or tumor fragment(s) or immortalized cells are inserted into the three-dimensional, engineered, tissue by injection, and then matured in cell culture media to allow the opening caused by the injection to close. In another embodiment, the tumor cells, tumor or tumor fragment(s) or immortalized cells are inserted by incision into the three-dimensional, engineered, tissue, and tumor cells tumor or tumor fragment(s) are inserted into the incision, and matured in cell culture media to allow the opening caused by the incision to close. In one embodiment, the plurality of stromal cell types are limited to human mammary fibroblasts, human endothelial cells, and human adipocytes. In another embodiment, the cancer cells, tumor or tumor fragment(s) are derived from a different patient than the plurality of stromal cell types.

In another embodiment, the invention provides methods of identifying a therapeutic agent for the treatment of cancer, the method comprising: contacting the three-dimensional, engineered biological cancer model with a candidate therapeutic agent; and measuring an effect on the cancer tumor, tumor fragment(s) or tumor cells. In one embodiment, the method is for identifying a therapeutic agent for the treatment of cancer in an individual and the tumor, tumor fragment(s) or tumor cells or immortalized cells are from that individual. In one embodiment, the effect is the extent, if any, of reduction in the size of the tumor or of the tumor growth. In another embodiment, the effect is the extent of apoptosis, the extent of damage, a reduced viability, the appearance, level or disappearance of tumor cell markers, appearance, level or disappearance of secreted cytokines, or the rate of proliferation of the tumor, tumor fragment(s), tumor cells or immortalized cells. In a further embodiment, if the therapeutic agent is effective against the tumor, tumor fragment, tumor cells or immortalized cells, the invention provides administering the therapeutic agent to the individual.

### Certain Definitions

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

A “non-human animal” may be any species other than human. In one embodiment, a non-human animal is a mammal. In another embodiment, a non-human animal is a vertebrate. In another embodiment, a non-human animal is selected from the group consisting of murine, ovine, canine, bovine, porcine and non-human primates.

As used herein “therapeutic agent” means any molecule, biologic, compound or composition that is approved to treat a disease, under investigation to treat a disease, or that elicits a biological response such as changes in DNA, RNA, peptide, polypeptide or protein.

As used herein, “tissue” means an aggregate of cells.

As used herein, “connective tissue cells derived from the mesoderm” refers to mesoderm derived cells that form connective tissue.

As used herein, “stroma” refers to the connective, supportive framework of a biological cell, tissue, or organ. In one embodiment, the stromal cells are primary stromal cells from a human. Commercially available stromal cells useful in the practice of the invention include WPMY-1 (ATCC® CRL-2854™), GMMe (ATCC® CRL-2674™), S1/S14 hSCF220 (ATCC® CRL-2453™), GMMs (ATCC® CRL-2675™), KMC8.8 (ATCC® CRL-2212™), KM114 (ATCC® TIB-242™), KM201 (ATCC® TIB-240™), KM703 (ATCC® CRL-1896™), KM81 (ATCC® TIB-241™), M2-10B4 (ATCC® CRL-1972™), RWPE-1 (ATCC® CRL-11609™), EML Cell Line, Clone 1 (ATCC® CRL-11691™), PWR-1E (ATCC® CRL-11611™), PS/2 (ATCC® CRL-1911™), M/K-1.9 (ATCC® CRL-1910™), M/K-2.7 (ATCC® CRL-1909™), KMI6 (ATCC® CRL-2179™), S1/S14 (ATCC® CRL-2452™), CCD-1086Sk (ATCC® CRL-2103™), Hs 574.T (ATCC® CRL-7345™), MD2 (ATCC® HB-229™), 5E10 (ATCC® CRL-2698™), CDR1 (ATCC® HB-213™), T HESCs (ATCC® CRL-4003™), S1/S14 hSCF248 (ATCC® CRL-2454™), AFT024 (ATCC® SCRC-1007™), D16 (ATCC® CRL-3281™), X9 (ATCC® CRL-3282™), D12 (ATCC® CRL-3280™), D1 ORL UVA (ATCC® CRL-12424™), HS-5 (ATCC® CRL-11882™), 7F2 (ATCC® CRL-12557™), W-20-17 (ATCC® CRL-2623™), 2E8 (ATCC® TIB-239™), HS-27A (ATCC® CRL-2496™), MYC 1-9E10.2 (ATCC® CRL-1729™), BEND (ATCC® CRL-2398™), VIII-6G10 (ATCC® HB-10519™), Primary Dermal Fibroblasts; Normal, Human, Adult (ATCC® PCS-201-012™), SR-4987 (ATCC® CRL-2028™), AFT024 IRR (ATCC® SCRC-1007.1™), WPE1-NB11 (ATCC® CRL-2851™), WPE1-NB26 (ATCC® CRL-2852™), WPE1-NB14 (ATCC® CRL-2850™), WPE1-NA22 (ATCC® CRL-2849™), WPE-stem (ATCC® CRL-2887™), RWPE2-W99 (ATCC® CRL-2853™), WPE1-NB26-64 (ATCC® CRL-2889™), WPE-int (ATCC® CRL-2888™), and WPE1-NB26-65 (ATCC® CRL-2890™) (American Type Culture Collection, Manassas, Va.).

Stromal cells can be derived from human-induced pluripotent stem cells via highly efficient, lineage-specific differentiation. One approach uses chemically defined media, feeder-free conditions, and a CD105 positive and CD24 negative selection to obtain single cell-based stromal cells derivation from differentiating human pluripotent cells in approximately 20 days. Lian et al., Methods in Molecular Biology 1416: 289-298 (2016).

Kidney stromal cells may be obtained from intermediate mesoderm (IM) with certain factors. Pietilä and Vainio, Nephron Exp. Nephrol. 126: 40-40 (2014). The stromal lineages controlling renal development derive from the intermediate mesoderm (IM). In addition, large populations of renal stromal cells also originate in the paraxial mesoderm. The signals that subdivide mesoderm into intermediate and paraxial domains may play a role in specifying renal stromal lineages. Guillaume et al., Developmental Biology 329: 169-175 (2009).

As used herein, “fibroblast-like cells” refers to cells having elongated fibrous structures which usually grow overlapping each other such as human lung cells MRCS. Fibroblast-like cell lines are distinguishable from epithelial-like cell lines which are identifiable by polar cuboidal cell structure usually growing in a monolayer.

Exemplary fibroblasts or fibroblasts-like cells that may be used in the practice of the invention include Primary Normal Bladder Fibroblast Cells (ATCC® PCS-420-013™), Primary Lung Fibroblasts (ATCC® PCS-201-013™), BJ (ATCC® CRL-2522™), Primary Dermal Fibroblasts (ATCC® PCS-201-011™), WI-38 (ATCC® CCL-75™), Primary Dermal Fibroblasts (ATCC® PCS-201-012™), Primary Dermal Fibroblast Normal (ATCC® PCS-201-010™), IRR-MRC-5 [irradiated MRC-5] (ATCC® 55-X™), IMR-90 (ATCC® CCL-186™), WPMY-1 (ATCC® CRL-2854™), HFF-1 (ATCC® SCRC-1041™), WI-38 VA-13 subline 2RA (ATCC® CCL-75.1™), HFF-1 IRR (ATCC® SCRC-1041.1™), BJ-5ta (ATCC® CRL-4001™), Hs27 (ATCC® CRL-1634™), MRC-5 (ATCC® CCL-171™), Detroit 551 (ATCC® CCL-110™), CCD-16Lu (ATCC® CCL-204™), CCD-19Lu (ATCC® CCL-210™), CCD-27Sk (ATCC® CRL-1475™), LL 47 (MaDo) (ATCC® CCL-135™), MRC-9 (ATCC® CCL-212™), Malme-3 (ATCC® HTB-102™), CCD-986Sk (ATCC® CRL-1947™), CCD-1079Sk (ATCC® CRL-2097™), CCD-1070Sk (ATCC® CRL-2091™), WS1 (ATCC® CRL-1502™), LL 24 (ATCC® CCL-151™), CCD-1064Sk (ATCC® CRL-2076™), Hs68 (ATCC® CRL-1635™), CCD-1059Sk (ATCC® CRL-2072™), CCD-33Lu (ATCC® CRL-1490™), LL 29 (AnHa) (ATCC® CCL-134™), HEL 299 (ATCC® CCL-137™), LL 97A (A1My) (ATCC® CCL-191™), HFL1 (ATCC® CCL-153™), CCD-1090Sk (ATCC® CRL-2106™), CHON-002 (ATCC® CRL-2847™), and C 211 (ATCC® CCL-123™) (American Type Culture Collection, Manassas, Va.).

“Myofibroblasts” are cells that are between a fibroblast and a smooth muscle in phenotype. In some embodiments, the myofibroblasts are intestinal tissue myofibroblasts. In various embodiments, the intestinal tissue myofibroblasts are derived from primary cells isolated from human intestine. In some embodiments, the myofibroblasts are dermal or vascular in origin. Myofibroblasts are available commercially, for example, the ATTC, and include WPMY-1 (ATCC® CRL-2854™).

As used herein, “adipocyte” (also known as a “lipocyte” or “fat cell”) refers to the cells that primarily compose adipose tissue, which are specialized in storing energy as fat.

As used herein, “preadipocyte” refers to any cell that can be stimulated to form adipocytes.

As used herein, “tissue” means an aggregate of cells.

The term “genetic marker” as used herein is a nucleotide sequence (e.g., in a chromosome) that that varies among different subjects. In some embodiments, the genetic marker can be a single nucleotide polymorphism, a restriction fragment length polymorphism, a microsatellite, a deletion of nucleotides, an addition of nucleotides, a substitution of nucleotides, a repeat or duplication of nucleotides, a translocation of nucleotides, and/or an aberrant or alternate splice site resulting in production of a truncated or extended form of a protein.

The terms “molecular marker,” “biomarker,” or “biological marker” are used interchangeably herein to refer to a molecule (e.g., a nucleic acid or protein) contained within a sample taken from an organism or other matter, the activity or expression of which can be measured and used to reveal certain characteristics about the respective source.

As used herein, “bio-ink” means a liquid, semi-solid, or solid composition for use in bioprinting. In some embodiments, bio-ink comprises cell solutions, cell aggregates, cell-comprising gels, multicellular bodies, or tissues. In some embodiments, the bio-ink additionally comprises non-cellular materials that provide specific biomechanical properties that enable bioprinting. In some embodiments the bio-ink comprises an extrusion compound.

As used herein, “bioprinting” means utilizing three-dimensional, precise deposition of cells (e.g., cell solutions, cell-containing gels, cell suspensions, cell concentrations, multicellular aggregates, multicellular bodies, etc.) via methodology that is compatible with an automated or semi-automated, computer-aided, three-dimensional prototyping device (e.g., a bioprinter).

As used herein, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and not able to be removed from the tissue without damage/destruction of said tissue. In further embodiments, decellularized tissue scaffolds include decellularized native tissues or decellularized cellular material generated by cultured cells in any manner; for example, cell layers that are allowed to die or are decellularized, leaving behind the ECM they produced while living. The term “scaffoldless,” therefore, is intended to imply that scaffold is not an integral part of the engineered tissue at the time of use, either having been removed or remaining as an inert component of the engineered tissue. “Scaffoldless” is used interchangeably with “scaffold-free” and “free of pre-formed scaffold.”

As used herein, “assay” means a procedure for testing or measuring the presence or activity of a substance (e.g., a chemical, molecule, biochemical, protein, hormone, or drug, etc.) in an organic or biologic sample (e.g., cell aggregate, tissue, organ, organism, etc.).

As used herein, an “array of cells” are cells that have been deposited in a pre-determined pattern. In one embodiment, the array of cells is a line of cells. In another embodiment, the array of cells is a planar array of cells in a pattern. In a further embodiment, an array of cells is a stacked array of planar arrays of cells in a pattern. Such arrays do not occur in nature.

In connection with assays, an “array” is a scientific tool including an association of multiple elements spatially arranged to allow a plurality of tests to be performed on a sample, one or more tests to be performed on a plurality of samples, or both. In some embodiments, the arrays are adapted for, or compatible with, screening methods and devices, including those associated with medium- or high-throughput screening. In further embodiments, an array allows a plurality of tests to be performed simultaneously. In further embodiments, an array allows a plurality of samples to be tested simultaneously. In some embodiments, the arrays are microarrays of the three-dimensional, engineered, biological cancer model. In further embodiments, the array is on the surface of a solid support. In other embodiments, the arrays are tissue microarrays. In further embodiments, arrays are assembled to allow the performance of multiple biochemical, metabolic, molecular, or histological analyses.

Exemplary multi-well plates that may be used to contain the tissue microarrays include Corning® CellBIND® cell culture multi-well plates, Corning® Costar® cell culture plates, Corning® Costar® Ultra-Low attachment multi-well plates, Corning® osteo assay surface multi-well plates, Corning® Synthemax®-R surface multi-well plates, Corning® Synthemax®-T surface multi-well plates, TPP® tissue culture plates, Greiner CELLSTAR® multi-well culture plates, Nunclon® Δ Multidishes, and Nunc® MicroWell® MiniTrays (Sigma-Aldrich, St. Louis, Mo.).

In some embodiments, the three-dimensional, engineered, biological cancer model exists in wells of a biocompatible multi-well container. In some embodiments, each model is placed into a well. In other embodiments, each model is added to a well by bioprinting the plurality of stromal cell types into a well, maturing the bioprinted construct, inserting an undissociated, primary, patient-derived cancer tumor, tumor fragment of tumor cells into the opening, and maturing the three-dimensional, engineered, biological cancer model to allow the opening to close. In further embodiments, the wells are coated. In various further embodiments, the wells are coated with one or more of: a biocompatible hydrogel, one or more proteins, one or more chemicals, one or more peptides, one or more antibodies, and one or more growth factors, including combinations thereof. In some embodiments, the wells are coated with NovoGel®. In other embodiments, the wells are coated with agarose. In some embodiments, each tissue exists on a porous, biocompatible membrane within a well of a biocompatible multi-well container. In some embodiments, each well of a multi-well container contains two or more tissues. In other embodiments, each cancer model comprises two or more tumor(s), tumor fragment(s), cells or immortalized cells in separate compartments.

In some embodiments, the three-dimensional, engineered, biological cancer model is secured to a biocompatible surface on one or more sides. Many methods are suitable to secure a tissue model to a biocompatible surface. In various embodiments, a tissue model is suitably secured to a biocompatible surface, for example, along one or more entire sides, only at the edges of one or more sides, or only at the center of one or more sides. In various further embodiments, a tissue model is suitably secured to a biocompatible surface with a holder or carrier integrated into the surface or associated with the surface. In various further embodiments, a tissue model is suitably secured to a biocompatible surface with one or more pinch-clamps or plastic nubs integrated into the surface or associated with the surface. In some embodiments, a tissue model is suitably secured to a biocompatible surface by cell-attachment to a porous membrane. In some embodiments, the three-dimensional, engineered, biological cancer model is held in an array configuration by affixation to a biocompatible surface on one or more sides. In further embodiments, the tissue model is affixed to a biocompatible surface on 1, 2, 3, 4, or more sides. In some embodiments, the biocompatible surface is any surface that does not pose a significant risk of injury or toxicity to the tissue model. In further embodiments, the biocompatible surface is any surface suitable for traditional tissue culture methods. Suitable biocompatible surfaces include, by way of non-limiting examples, treated plastics, membranes, porous membranes, coated membranes, coated plastics, metals, coated metals, glass, treated glass, and coated glass, wherein suitable coatings include hydrogels, ECM components, chemicals, proteins, etc., and coatings or treatments provide a means to stimulate or prevent cell and tissue adhesion to the biocompatible surface.

In some embodiments, the three-dimensional, engineered, biological cancer model comprises an association of two or more elements. In various embodiments, the arrays comprise an association of 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, or 500 elements, including increments therein. In further embodiments, each element comprises a three-dimensional, engineered, biological cancer model.

In some embodiments, the arrays of three-dimensional, engineered, biological cancer models comprise multiple elements spatially arranged in a pre-determined pattern. In further embodiments, the pattern is any suitable spatial arrangement of elements. In various embodiments, patterns of arrangement include, by way of non-limiting examples, a two-dimensional grid, a three-dimensional grid, one or more lines, arcs, or circles, a series of rows or columns, and the like. In further embodiments, the pattern is chosen for compatibility with medium- or high-throughput biological assay or screening methods or devices.

In some embodiments, each three-dimensional, engineered, biological cancer model within the array is maintained independently in culture. In further embodiments, the culture conditions of each model tissue within the array are such that they are isolated from the other tissues and cannot exchange media or factors soluble in the media. In further embodiments, the culture conditions of two or more individual tissue models within the array are such that they exchange media and factors soluble in the media with other tissues. In various embodiments, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, or more of the tissue models are within the array, including increments therein, exchange media and/or soluble factors. In other various embodiments, 1, 2, 3, 4, 5, 6, 7, 8, 9, 10, 15, 20, 25, 30, 35, 40, 45, 50, 55, 60, 65, 70, 75, 80, 85, 90, 91, 92, 93, 94, 95, 96, 97, 98, 99, or 100% of the tissue models within the array, including increments therein, exchange media and/or soluble factors.

In some embodiments, the three-dimensional, engineered, biological cancer model, described herein may be used in, by way of non-limiting examples, image-based assays, measurement of secreted proteins, expression of markers, and production of proteins. In various further embodiments, the three-dimensional, engineered, biological cancer model, described herein may be used in assays to detect or measure one or more of: molecular binding (including radioligand binding), molecular uptake, activity (e.g., enzymatic activity and receptor activity, etc.), gene expression, protein expression, receptor agonism, receptor antagonism, cell signaling, apoptosis, chemosensitivity, transfection, cell migration, chemotaxis, cell viability, cell proliferation, safety, efficacy, metabolism, toxicity, and abuse liability.

In some embodiments, the three-dimensional, engineered, biological cancer model, described herein may be used in immunoassays. In further embodiments, immunoassays are competitive immunoassays or noncompetitive immunoassays. In a competitive immunoassay, for example, the antigen in a sample competes with labeled antigen to bind with antibodies and the amount of labeled antigen bound to the antibody site is then measured. In a noncompetitive immunoassay (also referred to as a “sandwich assay”), for example, antigen in a sample is bound to an antibody site; subsequently, labeled antibody is bound to the antigen and the amount of labeled antibody on the site is then measured.

In some embodiments, three-dimensional, engineered, biological cancer model, described herein may be used in enzyme-linked immunosorbent assays (ELISA). In further embodiments, an ELISA is a biochemical technique used to detect the presence of an antibody or an antigen in a sample. In ELISA, for example, at least one antibody with specificity for a particular antigen is utilized. By way of further example, a sample with an unknown amount of antigen is immobilized on a solid support (e.g., a polystyrene microtiter plate) either non-specifically (via adsorption to the surface) or specifically (via capture by another antibody specific to the same antigen, in a “sandwich” ELISA). By way of still further example, after the antigen is immobilized, the detection antibody is added, forming a complex with the antigen. The detection antibody is, for example, covalently linked to an enzyme, or is itself detected by a secondary antibody that is linked to an enzyme through bioconjugation.

In some embodiments, an array of three-dimensional, engineered, biological cancer models may be used for drug screening, drug discovery or for identifying candidate therapeutic agents for the treatment of cancer. In further embodiments, an array of three-dimensional, engineered, biological models may be part of a kit for drug screening, drug discovery or personalized medicine. In some embodiments, each three-dimensional, engineered, biological cancer model may exist within a well of a biocompatible multi-well container, wherein the container is compatible with one or more automated drug screening procedures and/or devices. In further embodiments, automated drug screening procedures and/or devices include any suitable procedure or device that is computer or robot-assisted.

Provided is a method of identifying a therapeutic agent for the treatment of cancer, comprising

(a) contacting a candidate therapeutic agent with the three-dimensional, engineered biological cancer model described herein or a plurality of the three-dimensional, engineered, biological cancer models;

(b) measuring an effect on the primary tumor, primary tumor fragment(s), primary tumor cells or immortalized cells; and

(c) selecting the therapeutic agent for treatment of cancer based upon the measured effect.

In one embodiment, the method is for identifying a therapeutic agent for treatment of cancer in an individual and the tumor, tumor fragment(s), tumor cells or immortalized cells derived are from that individual. In another embodiment, the cancer is breast cancer, lung cancer, liver cancer, kidney cancer, prostate cancer, intestinal (e.g., colorectal) cancer, pancreatic cancer or skin cancer. In another embodiment, the cancer is gastric cancer, ovarian cancer, cervical cancer, uterine cancer, bladder cancer, esophageal cancer, or testicular cancer.

In one embodiment, the stromal cells are breast stromal cells, and the tumor, tumor fragment(s), tumor cells or immortalized cells are derived from an individual having breast cancer. In another embodiment, the stromal cells are lung stromal cells, and the tumor, tumor fragment(s), tumor cells or immortalized cells are derived from an individual having lung cancer. In another embodiment, the stromal cells are liver stromal cells, and the tumor, tumor fragment(s), tumor cells or immortalized cells are derived from an individual having liver cancer. In another embodiment, the stromal cells are kidney stromal cells, and the tumor, tumor fragment(s), tumor cells or immortalized cells are derived from an individual having kidney cancer. In another embodiment, the stromal cells are prostate stromal cells, and the tumor, tumor fragment(s), tumor cells or immortalized cells are derived from an individual having prostate cancer. In another embodiment, the stromal cells are intestinal (e.g., colorectal) stromal cells, and the tumor, tumor fragment(s), tumor cells or immortalized cells are derived from an individual having intestinal (e.g., colorectal) cancer. In another embodiment, the stromal cells are pancreatic cells, and the tumor, tumor fragment(s), tumor cells or immortalized cells are derived from an individual having pancreatic cancer. In another embodiment, the stromal cells are skin cells, and the tumor, tumor fragment(s), tumor cells or immortalized cells are derived from an individual having skin cancer. In another embodiment, the stromal cells are gastric stromal cells, and the tumor, tumor fragment(s), tumor cells or immortalized cells are derived from an individual having gastric cancer. In another embodiment, the stromal cells are ovarian stromal cells, and the tumor, tumor fragment(s), tumor cells or immortalized cells are derived from an individual having ovarian cancer. In another embodiment, the stromal cells are cervical stromal cells, and the tumor, tumor fragment(s), tumor cells or immortalized cells are derived from an individual having cervical cancer. In another embodiment, the stromal cells are uterine stromal cells, and the tumor, tumor fragment(s), tumor cells or immortalized cells are derived from an individual having uterine cancer. In another embodiment, the stromal cells are bladder stromal cells, and the tumor, tumor fragment(s), tumor cells or immortalized cells are derived from an individual having bladder cancer. In another embodiment, the stromal cells are esophageal stromal cells, and the tumor, tumor fragment(s), tumor cells or immortalized cells are derived from an individual having esophageal cancer. In another embodiment, the stromal cells are testicular stromal cells, and the tumor, tumor fragment(s), tumor cells or immortalized cells are derived from an individual having testicular cancer.

In another embodiment, each cancer model of the plurality of cancer models represent subtypes of a particular type of cancer.

In another embodiment, the cancer models are in culture media under static culture conditions. In another embodiment, the cancer models are in culture media under non-static culture conditions. In one embodiment, the non-static conditions is lateral flow across the cancer models. As disclosed in Example 8, it was unexpectedly discovered that lateral flow of culture media across the cancer models results in better models for testing drugs.

In another embodiment, the assay is carried out in a high throughput assay.

The effect on the primary tumor, primary tumor fragment(s), primary tumor cells or immortalized cells may be measured by any one or more of

(a) detecting any reduction of the size of the primary tumor or primary tumor fragment(s);

(b) detecting any reduction in the growth of the primary tumor, primary tumor fragment(s), primary tumor cells or immortalized cells;

(c) detecting apoptosis in the primary tumor, primary tumor fragment(s), primary tumor cells or immortalized cells;

(d) detecting the extent of damage of the primary tumor, primary tumor fragment(s), primary tumor cells or immortalized cells;

(e) detecting reduced viability of the primary tumor, primary tumor fragment(s), primary tumor cells or immortalized cells;

detecting the appearance or disappearance of cell markers on the primary tumor, primary tumor fragment(s), primary tumor cells or immortalized cells;

(g) detecting a change in the rate of proliferation or growth of the primary tumor, primary tumor fragment(s), primary tumor cells or immortalized cells;

(h) detecting a change in the staining of the primary tumor, primary tumor fragment(s), primary tumor cells or immortalized cells;

(i) detecting a change in RNA or DNA and/or level of expression in the primary tumor, primary tumor fragment(s), primary tumor cells or immortalized cells;

(j) detecting a change in protein expression in the primary tumor, primary tumor fragment(s), primary tumor cells or immortalized cells;

(k) detecting a change in cytokine expression or level in the primary tumor, primary tumor fragment(s), primary tumor cells or immortalized cells; or

(l) detecting T-cell recruitment, myeloid-lineage cell recruitment, infiltration and/or activation in the primary tumor, primary tumor fragment(s), primary tumor cells or immortalized cells according to methods that are well known.

Candidate therapeutic agents that may be tested with the three-dimensional, engineered, biological cancer model include anti-cancer agents that are either known or being tested for their anti-cancer properties. Examples of anti-cancer agents include, but are not limited to, an aromatase inhibitor; an anti-estrogen; an anti-androgen; a gonadorelin agonist; a topoisomerase I inhibitor; a topoisomerase II inhibitor; a microtubule active agent; an alkylating agent; a retinoid, a carontenoid, or a tocopherol; a cyclooxygenase inhibitor; an MMP inhibitor; an mTOR inhibitor; an antimetabolite; a platin compound; a methionine aminopeptidase inhibitor; a bisphosphonate; an antiproliferative antibody; a heparanase inhibitor; an inhibitor of Ras oncogenic isoforms; a telomerase inhibitor; a proteasome inhibitor; a compound used in the treatment of hematologic malignancies; a Flt-3 inhibitor; an Hsp90 inhibitor; a kinesin spindle protein inhibitor; a MEK inhibitor; an antitumor antibiotic; a nitrosourea; a compound targeting/decreasing protein or lipid kinase activity, a compound targeting/decreasing protein or lipid phosphatase activity, or any further anti-angiogenic compound.

Nonlimiting exemplary aromatase inhibitors include, but are not limited to, steroids, such as atamestane, exemestane, and formestane, and non-steroids, such as aminoglutethimide, roglethimide, pyridoglutethimide, trilostane, testolactone, ketokonazole, vorozole, fadrozole, anastrozole, and letrozole.

Nonlimiting anti-estrogens include, but are not limited to, tamoxifen, fulvestrant, raloxifene, and raloxifene hydrochloride. Anti-androgens include, but are not limited to, bicalutamide. Gonadorelin agonists include, but are not limited to, abarelix, goserelin, and goserelin acetate.

Exemplary topoisomerase I inhibitors include, but are not limited to, topotecan, gimatecan, irinotecan, camptothecin and its analogues, 9-nitrocamptothecin, and the macromolecular camptothecin conjugate PNU-166148. Topoisomerase II inhibitors include, but are not limited to, anthracyclines, such as doxorubicin, daunorubicin, epirubicin, idarubicin, and nemorubicin; anthraquinones, such as mitoxantrone and losoxantrone; and podophillotoxines, such as etoposide and teniposide.

Microtubule active agents include microtubule stabilizing, microtubule destabilizing compounds, and microtubulin polymerization inhibitors including, but not limited to, taxanes, such as paclitaxel and docetaxel; vinca alkaloids, such as vinblastine, vinblastine sulfate, vincristine, and vincristine sulfate, and vinorelbine; discodermolides; cochicine and epothilones and derivatives thereof.

Exemplary nonlimiting alkylating agents include cyclophosphamide, ifosfamide, melphalan, and nitrosoureas, such as carmustine and lomustine.

Exemplary nonlimiting cyclooxygenase inhibitors include Cox-2 inhibitors, 5-alkyl substituted 2-arylaminophenylacetic acid and derivatives, such as celecoxib, rofecoxib, etoricoxib, valdecoxib, or a 5-alkyl-2-arylaminophenylacetic acid, such as lumiracoxib.

Exemplary nonlimiting matrix metalloproteinase inhibitors (“MMP inhibitors”) include collagen peptidomimetic and nonpeptidomimetic inhibitors, tetracycline derivatives, batimastat, marimastat, prinomastat, metastat, BMS-279251, BAY 12-9566, TAA211, MMI270B, and AAJ996.

Exemplary nonlimiting mTOR inhibitors include compounds that inhibit the mammalian target of rapamycin (mTOR) and possess antiproliferative activity such as sirolimus, everolimus, CCI-779, and ABT578.

Exemplary nonlimiting antimetabolites include 5-fluorouracil (5-FU), capecitabine, gemcitabine, DNA demethylating compounds, such as 5-azacytidine and decitabine, methotrexate and edatrexate, and folic acid antagonists, such as pemetrexed.

Exemplary nonlimiting platin compounds include carboplatin, cis-platin, cisplatinum, and oxaliplatin.

Exemplary nonlimiting methionine aminopeptidase inhibitors include bengamide or a derivative thereof and PPI-2458.

Exemplary nonlimiting bisphosphonates include etridonic acid, clodronic acid, tiludronic acid, pamidronic acid, alendronic acid, ibandronic acid, risedronic acid, and zoledronic acid.

Exemplary nonlimiting antiproliferative antibodies include trastuzumab, trastuzumab-DM1, cetuximab, bevacizumab, rituximab, PR064553, and 2C4. The term “antibody” is meant to include intact monoclonal antibodies, polyclonal antibodies, multispecific antibodies formed from at least two intact antibodies, and antibody fragments, so long as they exhibit the desired biological activity.

Exemplary nonlimiting heparanase inhibitors include compounds that target, decrease, or inhibit heparin sulfate degradation, such as PI-88 and OGT2115.

The term “an inhibitor of Ras oncogenic isoforms,” such as H-Ras, K-Ras, or N-Ras, as used herein refers to a compound which targets, decreases, or inhibits the oncogenic activity of Ras, for example, a farnesyl transferase inhibitor, such as L-744832, DK8G557, tipifarnib, and lonafarnib.

Exemplary nonlimiting telomerase inhibitors include compounds that target, decrease, or inhibit the activity of telomerase, such as compounds that inhibit the telomerase receptor, such as telomestatin.

Exemplary nonlimiting proteasome inhibitors include compounds that target, decrease, or inhibit the activity of the proteasome including, but not limited to, bortezomid.

The phrase “compounds used in the treatment of hematologic malignancies” as used herein includes FMS-like tyrosine kinase inhibitors, which are compounds targeting, decreasing or inhibiting the activity of FMS-like tyrosine kinase receptors (Flt-3R); interferon, I-β-D-arabinofuransylcytosine (ara-c), and bisulfan; and ALK inhibitors, which are compounds which target, decrease, or inhibit anaplastic lymphoma kinase.

Exemplary nonlimiting Flt-3 inhibitors include PKC412, midostaurin, a staurosporine derivative, SU11248, and MLN518.

Exemplary nonlimiting HSP90 inhibitors include compounds targeting, decreasing, or inhibiting the intrinsic ATPase activity of HSP90; or degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins, or antibodies that inhibit the ATPase activity of HSP90, such as 17-allylamino,17-demethoxygeldanamycin (17AAG), a geldanamycin derivative; other geldanamycin related compounds; radicicol and HDAC inhibitors.

A compound targeting/decreasing a protein or lipid kinase activity; or a protein or lipid phosphatase activity; or any further anti-angiogenic compound includes a protein tyrosine kinase and/or serine and/or threonine kinase inhibitor or lipid kinase inhibitor, such as a) a compound targeting, decreasing, or inhibiting the activity of the platelet-derived growth factor-receptors (PDGFR), such as a compound that targets, decreases, or inhibits the activity of PDGFR, such as an N-phenyl-2-pyrimidine-amine derivatives, such as imatinib, SU101, SU6668, and GFB-111; b) a compound targeting, decreasing, or inhibiting the activity of the fibroblast growth factor-receptors (FGFR); c) a compound targeting, decreasing, or inhibiting the activity of the insulin-like growth factor receptor I (IGF-IR), such as a compound that targets, decreases, or inhibits the activity of IGF-IR; d) a compound targeting, decreasing, or inhibiting the activity of the Trk receptor tyrosine kinase family, or ephrin B4 inhibitors; e) a compound targeting, decreasing, or inhibiting the activity of the Axl receptor tyrosine kinase family; f) a compound targeting, decreasing, or inhibiting the activity of the Ret receptor tyrosine kinase; g) a compound targeting, decreasing, or inhibiting the activity of the Kit/SCFR receptor tyrosine kinase, such as imatinib; h) a compound targeting, decreasing, or inhibiting the activity of the c-Kit receptor tyrosine kinases, such as imatinib; i) a compound targeting, decreasing, or inhibiting the activity of members of the c-Abl family, their gene-fusion products (e.g. Bcr-Abl kinase) and mutants, such as an N-phenyl-2-pyrimidine-amine derivative, such as imatinib or nilotinib; PD180970; AG957; NSC 680410; PD173955; or dasatinib; j) a compound targeting, decreasing, or inhibiting the activity of members of the protein kinase C (PKC) and Raf family of serine/threonine kinases, members of the MEK, SRC, JAK, FAK, PDK1, PKB/Akt, and Ras/MAPK family members, and/or members of the cyclin-dependent kinase family (CDK), such as a staurosporine derivative disclosed in U.S. Pat. No. 5,093,330, such as midostaurin; examples of further compounds include UCN-01, safingol, BAY 43-9006, bryostatin 1, perifosine; ilmofosine; RO 318220 and RO 320432; GO 6976; Isis 3521; LY333531/LY379196; a isochinoline compound; a farnesyl transferase inhibitor; PD184352 or QAN697, or AT7519; k) a compound targeting, decreasing or inhibiting the activity of a protein-tyrosine kinase, such as imatinib mesylate or a tyrphostin, such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) a compound targeting, decreasing, or inhibiting the activity of the epidermal growth factor family of receptor tyrosine kinases (EGFR, ErbB2, ErbB3, ErbB4 as homo- or heterodimers) and their mutants, such as CP 358774, ZD 1839, ZM 105180; trastuzumab, cetuximab, gefitinib, erlotinib, OSI-774, C1-1033, EKB-569, GW-2016, antibodies E1.1, E2.4, E2.5, E6.2, E6.4, E2.11, E6.3 and E7.6.3, and 7H-pyrrolo-[2,3-d]pyrimidine derivatives; and m) a compound targeting, decreasing, or inhibiting the activity of the c-Met receptor.

Exemplary compounds that target, decrease, or inhibit the activity of a protein or lipid phosphatase include inhibitors of phosphatase 1, phosphatase 2A, or CDC25, such as okadaic acid or a derivative thereof.

Further anti-angiogenic compounds include compounds having another mechanism for their activity unrelated to protein or lipid kinase inhibition, e.g., thalidomide and TNP-470.

Additional, nonlimiting, candidate therapeutic agents include: daunorubicin, adriamycin, Ara-C, VP-16, teniposide, mitoxantrone, idarubicin, carboplatinum, PKC412, 6-mercaptopurine (6-MP), fludarabine phosphate, octreotide, SOM230, FTY720, 6-thioguanine, cladribine, 6-mercaptopurine, pentostatin, hydroxyurea, 2-hydroxy-1H-isoindole-1,3-dione derivatives, l-(4-chloroanilino)-4-(4-pyridylmethyl)phthalazine or a pharmaceutically acceptable salt thereof, 1-(4-chloroanilino)-4-(4-pyridylmethyl)phthalazine succinate, angiostatin, endostatin, anthranilic acid amides, ZD4190, ZD6474, SU5416, SU6668, bevacizumab, rhuMAb, rhuFab, macugon; FLT-4 inhibitors, FLT-3 inhibitors, VEGFR-2 IgGI antibody, RPI 4610, bevacizumab, porfimer sodium, anecortave, triamcinolone, hydrocortisone, 11-a-epihydrocotisol, cortex olone, 17a-hydroxyprogesterone, corticosterone, desoxycorticosterone, testosterone, estrone, dexamethasone, fluocinolone, a plant alkaloid, a hormonal compound and/or antagonist, a biological response modifier, such as a lymphokine or interferon, an antisense oligonucleotide or oligonucleotide derivative, shRNA, and siRNA.

Additional, nonlimiting, candidate therapeutic agents include immunotherapies, such as adoptive T cell transfer, immune checkpoint inhibitors to activate Tc and NK cells, and immune cell reprogramming and depletion. In adoptive T-cell transfer, nonlimiting examples of candidate therapeutic agents include genetically modified T cells, such as T cells with gene transfer of a synthetic TCR or a chimeric antigen receptor (CAR) that is capable of activating T cell continuously. Adoptive transfer of T cells genetically modified to express CAR receptors can be used for targeting CD19 for B-ALL, CLL and MM. For immune checkpoint inhibitors to activate Tc and NK cells, nonlimiting examples of candidate therapeutic agents include anti-CTLA-4 (i.e., ipilimumab (Yervoy)), anti-PD-L1 (i.e., pembrolizumab (Keytruda)), anti-PD1 (i.e., nivolumab (Opdivo)), and anti-KIR (i.e., lirulumab). For immune cell reprogramming and depletion, nonlimiting examples of candidate therapeutic agents include anti-CSF1R, anti-CXCR4 (i.e., AMD3100 (Plerixafor)), anti-CXCR2 (i.e. AZD5069), indoleamine 2,3-dioxygenase (i.e., IDO1) inhibitor (Epacadostat)), and arginase inhibitor (i.e., CB-1158)).

Cancers that may be treated with the candidate therapeutic agents include but are not limited to, adrenal cancer, acinic cell carcinoma, acoustic neuroma, acral lentigious melanoma, acrospiroma, acute eosinophilic leukemia, acute erythroid leukemia, acute lymphoblastic leukemia, acute megakaryoblastic leukemia, acute monocytic leukemia, acute promyelocytic leukemia, adenocarcinoma, adenoid cystic carcinoma, adenoma, adenomatoid odontogenic tumor, adenosquamous carcinoma, adipose tissue neoplasm, adrenocortical carcinoma, adult T-cell leukemia/lymphoma, aggressive NK-cell leukemia, AIDS-related lymphoma, alveolar rhabdomyosarcoma, alveolar soft part sarcoma, ameloblastic fibroma, anaplastic large cell lymphoma, anaplastic thyroid cancer, angioimmunoblastic T-cell lymphoma, angiomyolipoma, angiosarcoma, astrocytoma, atypical teratoid rhabdoid tumor, B-cell chronic lymphocytic leukemia, B-cell prolymphocytic leukemia, B-cell lymphoma, basal cell carcinoma, biliary tract cancer, bladder cancer, blastoma, bone cancer, Brenner tumor, Brown tumor, Burkitt's lymphoma, breast cancer, brain cancer, carcinoma, carcinoma in situ, carcinosarcoma, cartilage tumor, cementoma, myeloid sarcoma, chondroma, chordoma, choriocarcinoma, choroid plexus papilloma, clear-cell sarcoma of the kidney, craniopharyngioma, cutaneous T-cell lymphoma, cervical cancer, colorectal cancer, Degos disease, desmoplastic small round cell tumor, diffuse large B-cell lymphoma, dysembryoplastic neuroepithelial tumor, dysgerminoma, embryonal carcinoma, endocrine gland neoplasm, endodermal sinus tumor, enteropathy-associated T-cell lymphoma, esophageal cancer, fetus in fetu, fibroma, fibrosarcoma, follicular lymphoma, follicular thyroid cancer, ganglioneuroma, gastrointestinal cancer, germ cell tumor, gestational choriocarcinoma, giant cell fibroblastoma, giant cell tumor of the bone, glial tumor, glioblastoma multiforme, glioma, gliomatosis cerebri, glucagonoma, gonadoblastoma, granulosa cell tumor, gynandroblastoma, gallbladder cancer, gastric cancer, hairy cell leukemia, hemangioblastoma, head and neck cancer, hemangiopericytoma, hematological malignancy, hepatoblastoma, hepatosplenic T-cell lymphoma, Hodgkin's lymphoma, non-Hodgkin's lymphoma, invasive lobular carcinoma, intestinal cancer, kidney cancer, laryngeal cancer, lentigo maligna, lethal midline carcinoma, leukemia, leydig cell tumor, liposarcoma, lung cancer, lymphangioma, lymphangiosarcoma, lymphoepithelioma, lymphoma, acute lymphocytic leukemia, acute myelogeous leukemia, chronic lymphocytic leukemia, liver cancer, small cell lung cancer, non-small cell lung cancer, MALT lymphoma, malignant fibrous histiocytoma, malignant peripheral nerve sheath tumor, malignant triton tumor, mantle cell lymphoma, marginal zone B-cell lymphoma, mast cell leukemia, mediastinal germ cell tumor, medullary carcinoma of the breast, medullary thyroid cancer, medulloblastoma, melanoma, meningioma, merkel cell cancer, mesothelioma, metastatic urothelial carcinoma, mixed Mullerian tumor, mucinous tumor, multiple myeloma, muscle tissue neoplasm, mycosis fungoides, myxoid liposarcoma, myxoma, myxosarcoma, nasopharyngeal carcinoma, neurinoma, neuroblastoma, neurofibroma, neuroma, nodular melanoma, ocular cancer, oligoastrocytoma, oligodendroglioma, oncocytoma, optic nerve sheath meningioma, optic nerve tumor, oral cancer, osteosarcoma, ovarian cancer, Pancoast tumor, papillary thyroid cancer, paraganglioma, pinealoblastoma, pineocytoma, pituicytoma, pituitary adenoma, pituitary tumor, plasmacytoma, polyembryoma, precursor T-lymphoblastic lymphoma, primary central nervous system lymphoma, primary effusion lymphoma, preimary peritoneal cancer, prostate cancer, pancreatic cancer, pharyngeal cancer, pseudomyxoma periotonei, renal cell carcinoma, renal medullary carcinoma, retinoblastoma, rhabdomyoma, rhabdomyosarcoma, Richter's transformation, rectal cancer, sarcoma, Schwannomatosis, seminoma, Sertoli cell tumor, sex cord-gonadal stromal tumor, signet ring cell carcinoma, skin cancer, small blue round cell tumors, small cell carcinoma, soft tissue sarcoma, somatostatinoma, soot wart, spinal tumor, splenic marginal zone lymphoma, squamous cell carcinoma, synovial sarcoma, Sezary's disease, small intestine cancer, squamous carcinoma, stomach cancer, T-cell lymphoma, testicular cancer, thecoma, thyroid cancer, transitional cell carcinoma, throat cancer, urachal cancer, urogenital cancer, urothelial carcinoma, uveal melanoma, uterine cancer, verrucous carcinoma, visual pathway glioma, vulvar cancer, vaginal cancer, Waldenstrom's macroglobulinemia, Warthin's tumor, and Wilms' tumor. Any tumor(s), tumor fragment(s), tumor cells or immortalized cells from the aforementioned cancers may be employed in the cancer models described herein.

### Methods

The ex vivo system described herein comprises a bioprinted three-dimensional dense stromal capsule or stromal block. Fibroblasts on the outer surface of the tissue facilitate incision and implantation of the tumor, tumor fragment(s), tumor cells or immortalized cells. The human stromal environment allows the retention of immune cells, patient-specific tumor cells, and/or recruitment and retention of immune cells to facilitate the testing of immune-based therapies on the tumor cells.

Accordingly, in some embodiments, the methods of fabricating three-dimensional, engineered, biological cancer models comprise preparing a bio-ink comprising connective tissue cells derived from the mesoderm. In further embodiments, the stromal bio-ink comprises one or more stromal cell types. In still further embodiments, the stromal cell types comprise one or more of: human mammary fibroblasts, human endothelial cells, and human adipocytes, preadipocytes, or a combination of adipocytes and preadipocytes. In some embodiments, the stromal bio-ink comprises one or more extrusion compounds. In various embodiments, suitable extrusion compounds include, by way of non-limiting examples, alginate, hydrogel, Novogel, Matrigel, extracellular matrix components, and the like. In various further embodiments, suitable extrusion compounds include, by way of non-limiting examples, water soluble, cross-linkable, and biodegradable polymers, gels, and the like. In one embodiment, the extrusion compounds are cross-linked by exposure to UV radiation before, during or after the extrusion compound is extruded. See, US2014/0093932. In another embodiment, the cross-linked extrusion compound is removed by enzymatic degradation subsequent to cell cohesion. For example, when the extrusion compound is alginate, it may be removed by incubation in the presence of alginate lyase.

The stromal bio-ink is suitably prepared and deposited to form a stacked array of cells and matured to form three-dimensional, engineered stromal tissues that recapitulate, to a degree, native stromal tissues. The engineered stromal tissues have structural integrity sufficient to tolerate manipulation, injection and incision. And, many engineered epithelial stromal tissues are suitable for use in the methodologies described herein.

Bioprinted constructs are made by a method that utilizes a rapid prototyping technology based on three-dimensional, automated, computer-aided deposition of an array of cells, including cell solutions, cell suspensions, cell-comprising gels or pastes, cell concentrations, multicellular bodies (e.g., cylinders, spheroids, ribbons, etc.) and, optionally, confinement material onto a biocompatible surface (e.g., composed of hydrogel and/or a porous membrane) by a three-dimensional delivery device (e.g., a bioprinter). As used herein, in some embodiments, the term “engineered,” when used to refer to tissues, means that cells, cell solutions, cell suspensions, cell-comprising gels or pastes, cell concentrates, multicellular aggregates, and layers thereof are positioned in an array to form three-dimensional structures by a computer-aided device (e.g., a bioprinter) according to a computer script. In further embodiments, the computer script is, for example, one or more computer programs, computer applications, or computer modules. In still further embodiments, three-dimensional tissue structures form through the post-printing fusion of cells or multicellular bodies which, in some cases, is similar to self-assembly phenomena in early morphogenesis.

While a number of methods are available to arrange cells, multicellular aggregates, and/or layers thereof on a biocompatible surface to produce a three-dimensional structure including manual placement, positioning into an array by an automated, computer-aided machine such as a bioprinter is advantageous. Advantages of delivery of cells or multicellular bodies with this technology include rapid, accurate, and reproducible placement of cells or multicellular bodies to produce constructs exhibiting planned or pre-determined array of cells, multicellular aggregates and/or layers thereof with various compositions. Advantages also include assured high cell density, while minimizing cell damage.

In some embodiments, the method of bioprinting is continuous and/or substantially continuous. A non-limiting example of a continuous bioprinting method is to dispense bio-ink (i.e., cells, cells combined with an excipient or extrusion compound, or aggregates of cells) from a bioprinter via a dispense tip (e.g., a syringe, needle, capillary tube, etc.) connected to a reservoir of bio-ink. In further non-limiting embodiments, a continuous bioprinting method dispenses bio-ink in a repeating pattern of functional units. In various embodiments, a repeating functional unit has any suitable geometry, including, for example, circles, squares, rectangles, triangles, polygons, and irregular geometries, thereby resulting in one or more tissue layers with planar geometry achieved via spatial patterning of distinct bio-inks and/or void spaces. In further embodiments, a repeating pattern of bioprinted functional units comprises a plurality of layers are bioprinted adjacently (e.g., stacked) to form an engineered tissue or organ with laminar geometry. In various embodiments, 2, 3, 4, 5, 6, 7, 8, 9, 10, 11, 12, 13, 14, 15, or more layers are bioprinted adjacently (e.g., stacked) to form an engineered tissue. In further embodiments, one or more layers of a tissue with laminar geometry also has planar geometry.

In some embodiments, a bioprinted functional unit repeats in a tessellated pattern. A “tessellated pattern” is a plane of figures that fills the plane with no overlaps and no gaps. Advantages of continuous and/or tessellated bioprinting includes, by way of a non-limiting example, increased productivity of bioprinted tissue. Another non-limiting, exemplary advantage is eliminating the need to align the bioprinter with previously deposited elements of bio-ink. In some embodiments, continuous bioprinting facilitates printing larger tissues from a large reservoir of bio-ink, optionally using a syringe mechanism. Continuous bioprinting is also a convenient way to co-print spatially-defined boundaries, using an extrusion compound, a hydrogel, a polymer, bio-ink, or any printable material that is capable of retaining its shape post-printing; wherein the boundaries that are created are optionally filled in via the bioprinting of a one or more bio-inks, thereby creating a mosaic tissue with spatially-defined planar geometry.

In some embodiments, the stromal bio-ink is deposited by a bioprinter apparatus. Suitable bioprinters include the Novogen Bioprinter® (Organovo, Inc., San Diego, Calif.). In some embodiments, the bioprinting comprises extrusion of a solid or semi-solid bio-ink onto a surface to give a stacked array layer-by-layer.

Many shapes and sizes of bioprinted tissue are suitable. By way of example, in various embodiments, suitable shapes of bioprinted tissue include planar, rectangular, cuboidal, triangular, pyramidal, pentagonal, hexagonal, cylindrical, spheroidal, ovoid, and irregular. By way of example, in various embodiments, suitable sizes of bioprinted tissue include about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μm, or more, including increments therein. By way of further example, in various embodiments, suitable sizes of bioprinted tissue include about 1, 2, 3, 4, 5, 6, 7, 8, 9, 10, 11, 12, 13, 14, 15, 16, 17, 18, 19, 20 mm, or more, including increments therein. In light of the disclosure provided herein, those of skill the art will recognize that a minimum size is, in some embodiments, determined, at least in part, by the available area necessary to incise or inject the tissue on a surface to form an opening. Also, in light of the disclosure provided herein, those of skill the art will recognize that a maximum size is, in some embodiments, determined, at least in part, by the ability of nutrients and gasses to reach the center of the tissue.

In some embodiments, the methods of fabricating three-dimensional, engineered, biological cancer models comprise maturing the deposited bio-ink in a cell culture media. In one embodiment, the cell culture media is a eukaryotic cell culture media. Examples of eukaryotic cell culture media that may be used in the practice of the invention include BGJb, BME, Brinster's BMOC-3, CMRL, CO2-Independent Medium, DMEM Media, DMEM/F-12 Media, F-10 Nutrient Mixture, F-12 Nutrient Mixture, Glasgow (G-MEM), Improved MEM, Iscove's (IMDM), Leibovitz's L-15, McCoy's 5A, MCDB 131, Media 199, Minimum Essential Media (MEM), Modified Eagle Medium (MEM), Opti-MEM® I, Fischer's Medium, MEM Rega-3, NCTC-135 Medium, RPMI Medium 1640, Waymouth's MB 752/1, and Williams' Media E (ThermoFisher Scientific, Grand Island, New York). In further embodiments, maturation in cell culture allows the cells to cohere to form a three-dimensional, engineered, stromal tissue. In still further embodiments, maturation in cell culture media facilitates removal of any extrusion compound.

In some embodiments, the engineered stromal tissues are conditioned under non-static culture conditions. Stromal tissues preconditioned under non-static conditions exhibit a dense capsule of fibroblasts on the outer surface of the tissue that facilitates injection by a needle and/or incision with a scalpel.

Non-static conditions include those in which the liquid cell culture is in motion. Non-static conditions may be induced using spinner flasks (stirred suspension), roller bottles, perfusion, aeration, stirred, or rotated, such as in rotating wall vessels or rotary cell culture systems. Examples of spinner flasks include, but are not limited to, Corning® ProCulture spinner flasks and Corning® disposable spinner flasks (Sigma-Aldrich, St. Louis, Mo.). Examples of roller bottles include, but are not limited to, Corning® Roller Bottles CellBIND® Cell Culture Surface and Corning® Roller Bottles Tissue Culture Treated (Sigma-Aldrich, St. Louis, Mo.). Examples of rotating wall vessels include, but are not limited to, Disposable HARVs, HARVs and STLVs (Synthecon, Houston, Tex.).

Shear stress is the mechanical force induced by the friction of liquid against a distal cell membrane. Shear stress may include unidirectional laminar flow, pulsatile laminar flow, turbulent flow, oscillating flow, and non-uniform laminar shear stress. Examples of the laminar shear stress include, but are not limited to, less than 28 dynes/cm², 5-25 dynes/cm², 6-25 dynes/cm², 7-25 dynes/cm², 8-25 dynes/cm², 9-25 dynes/cm², 10-25 dynes/cm², 11-25 dynes/cm², 12-25 dynes/cm², 13-25 dynes/cm², 14-25 dynes/cm², 15-25 dynes/cm², 16-25 dynes/cm², 17-25 dynes/cm², 18-25 dynes/cm², 19-25 dynes/cm², 20-25 dynes/cm², 21-25 dynes/cm², 22-25 dynes/cm², 23-25 dynes/cm², 24-25 dynes/cm², 5-24 dynes/cm², 6-24 dynes/cm², 7-24 dynes/cm², 8-24 dynes/cm², 9-24 dynes/cm², 10-24 dynes/cm², 11-24 dynes/cm², 12-24 dynes/cm², 13-24 dynes/cm², 14-24 dynes/cm², 15-24 dynes/cm², 16-24 dynes/cm², 17-24 dynes/cm², 18-24 dynes/cm², 19-24 dynes/cm², 20-24 dynes/cm², 21-24 dynes/cm², 22-24 dynes/cm², 23-24 dynes/cm², 5-23 dynes/cm², 6-23 dynes/cm², 7-23 dynes/cm², 8-23 dynes/cm², 9-23 dynes/cm², 10-23 dynes/cm², 11-23 dynes/cm², 12-23 dynes/cm², 13-23 dynes/cm², 14-23 dynes/cm², 15-23 dynes/cm², 16-23 dynes/cm², 17-23 dynes/cm², 18-23 dynes/cm², 19-23 dynes/cm², 20-23 dynes/cm², 21-23 dynes/cm², 22-23 dynes/cm², 5-22 dynes/cm², 6-22 dynes/cm², 7-22 dynes/cm², 8-22 dynes/cm², 9-22 dynes/cm², 10-22 dynes/cm², 11-22 dynes/cm², 12-22 dynes/cm², 13-22 dynes/cm², 14-22 dynes/cm², 15-22 dynes/cm², 16-22 dynes/cm², 17-22 dynes/cm², 18-22 dynes/cm², 19-22 dynes/cm², 20-22 dynes/cm², 21-22 dynes/cm², 5-21 dynes/cm², 6-21 dynes/cm², 7-21 dynes/cm², 8-21 dynes/cm², 9-21 dynes/cm², 10-21 dynes/cm², 11-21 dynes/cm², 12-21 dynes/cm², 13-21 dynes/cm², 14-21 dynes/cm², 15-21 dynes/cm², 16-21 dynes/cm², 17-21 dynes/cm², 18-21 dynes/cm², 19-21 dynes/cm², 20-21 dynes/cm², 5-20 dynes/cm², 6-20 dynes/cm², 7-20 dynes/cm², 8-20 dynes/cm², 9-20 dynes/cm², 10-20 dynes/cm², 11-20 dynes/cm², 12-20 dynes/cm², 13-20 dynes/cm², 14-20 dynes/cm², 15-20 dynes/cm², 16-20 dynes/cm², 17-20 dynes/cm², 18-20 dynes/cm², 19-20 dynes/cm², 5-19 dynes/cm², 6-19 dynes/cm², 7-19 dynes/cm², 8-19 dynes/cm², 9-19 dynes/cm², 10-19 dynes/cm², 11-19 dynes/cm², 12-19 dynes/cm², 13-19 dynes/cm², 14-19 dynes/cm², 15-19 dynes/cm², 16-19 dynes/cm², 17-19 dynes/cm², 18-19 dynes/cm², 5-18 dynes/cm², 6-18 dynes/cm², 7-18 dynes/cm², 8-18 dynes/cm², 9-18 dynes/cm², 10-18 dynes/cm², 11-18 dynes/cm², 12-18 dynes/cm², 13-18 dynes/cm², 14-18 dynes/cm², 15-18 dynes/cm², 16-18 dynes/cm², 17-18 dynes/cm², 5-17 dynes/cm², 6-17 dynes/cm², 7-17 dynes/cm², 8-17 dynes/cm², 9-17 dynes/cm², 10-17 dynes/cm², 11-17 dynes/cm², 12-17 dynes/cm², 13-17 dynes/cm², 14-17 dynes/cm², 15-17 dynes/cm², 16-17 dynes/cm², 5-16 dynes/cm², 6-16 dynes/cm², 7-16 dynes/cm², 8-16 dynes/cm², 9-16 dynes/cm², 10-16 dynes/cm², 11-16 dynes/cm², 12-16 dynes/cm², 13-16 dynes/cm², 14-16 dynes/cm², 15-16 dynes/cm², 5-15 dynes/cm², 6-15 dynes/cm², 7-15 dynes/cm², 8-15 dynes/cm², 9-15 dynes/cm², 10-15 dynes/cm², 11-15 dynes/cm², 12-15 dynes/cm², 13-15 dynes/cm², 14-15 dynes/cm², 5-14 dynes/cm², 6-14 dynes/cm², 7-14 dynes/cm², 8-14 dynes/cm², 9-14 dynes/cm², 10-14 dynes/cm², 11-14 dynes/cm², 12-14 dynes/cm², 13-14 dynes/cm², 5-13 dynes/cm², 6-13 dynes/cm², 7-13 dynes/cm², 8-13 dynes/cm², 9-13 dynes/cm², 10-13 dynes/cm², 11-13 dynes/cm², 12-13 dynes/cm², 5-12 dynes/cm², 6-12 dynes/cm², 7-12 dynes/cm², 8-12 dynes/cm², 9-12 dynes/cm², 10-12 dynes/cm², 11-12 dynes/cm², 5-11 dynes/cm², 6-11 dynes/cm², 7-11 dynes/cm², 8-11 dynes/cm², 9-11 dynes/cm², 10-11 dynes/cm², 5-10 dynes/cm², 6-10 dynes/cm², 7-10 dynes/cm², 8-10 dynes/cm², 9-10 dynes/cm², 5-9 dynes/cm², 6-9 dynes/cm², 7-9 dynes/cm², 8-9 dynes/cm², 5-8 dynes/cm², 6-8 dynes/cm², 7-8 dynes/cm², 5-7 dynes/cm², 6-7 dynes/cm², and 5-6 dynes/cm². See, Qui et al., Journal of The Royal Society Interface 11: 20130852 (2013).

Shear stress within a culture well may be measured using a MicroS3.v10 probe (Viosense Corporation, Pasadena, Calif.). The MicroS3 probe uses optical Doppler velocimetry to measure shear stress within 166 μm of its surface. The probe is mounted, and measurements are taken 1 mm from the center point of the well to sample the shear stress present in the center of the well, as well as 12 mm from the center point to sample the shear stress present in the periphery. The probe is mounted through a hole cut through the bottom of the well such that the tip of the probe is aligned flush with the bottom surface and thus measured shear stress at the level of a seeded cell. Shear stress=fDoppler·K·μ where fDoppler is the mean frequency (Hz) of the Doppler shift in the area sampled by the sensor and is calculated by Fast-Fourier Transformation; K is the fringe divergence, a constant characterized for each sensor; and μ is the dynamic viscosity and is equal to the product of the kinematic viscosity (υ) and the density (φ. The Reynolds' number is calculated as ωR2/υ where ω is the rotational speed of the orbital shaker, R is the radius of rotation of the orbital shaker, and υ is the kinematic viscosity. The well and attached probe are mounted on the surface of the orbital shaker, which is then adjusted from 60 to 210 rpm, and shear stress was measured at 30-rpm intervals; approximately 100 independent measurements were taken at each point. (Dardik et al., J. Vasc. Surg. 41: 869-880 (2005)).

In some embodiments, one or more preadipocyte differentiation signals are provided to the preadipocytes to effect differentiation to adipocytes. The preadipocyte differentiation signal may be provided during the maturing of the engineered stromal tissues or thereafter. Examples of preadipocyte differentiation signals include 1-isobutyl-3-methylxanthine, dexamethasone, insulin and mixtures thereof (Qui et al., J. Biol. Chem. 276:11988-11995 (2001), calpain (Patel and Lane, J. Biol. Chem. 275:17653-17660 (2000), glucocorticoids, 3,3′,5-triiodothyronine, retinoic acid, growth hormone, insulin-like growth factor I, epidermal growth factor, transforming growth factor, fibroblast growth factor, platelet-derived growth factor, interleukin-1, interferon-α, tumor necrosis factor-α, prostaglandin-F_{2α}, prostaglandin-I₂, forskolinm dibutyryl cAMPL, and 12-O-tetradecanoylphorbol 13-acetate (Gregoire, et al., Phys. Rev. 78(3):783-809 (1998)).

### Bio-Ink

In some embodiments, the bio-ink is produced by collecting a plurality of cells in a fixed volume; wherein the cellular component(s) represent at least about 30% and at most about 100% of the total volume. In some embodiments, bio-ink comprises semi-solid or solid multicellular aggregates or multicellular bodies. In further embodiments, the bio-ink is produced by 1) mixing a plurality of cells or cell aggregates and a biocompatible liquid or gel in a pre-determined ratio to result in bio-ink, and 2) compacting the bio-ink to produce the bio-ink with a desired cell density and viscosity. In some embodiments, the compacting of the bio-ink is achieved by centrifugation, tangential flow filtration (“TFF”), or a combination thereof.

In some embodiments, the bio-inks disclosed herein are characterized by high cellularity by volume, e.g., a high concentration of living cells. In further embodiments, the bio-ink comprise at least about 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400 or more million cells per milliliter of solution. In a particular embodiment, the bio-inks comprise about 50 to about 300 million cells/mL. In some embodiments, bio-inks that have high cellularity by volume are used to bioprint engineered tissues and constructs with high cell density. In further embodiments, the engineered tissues and constructs are at least about 30, 35, 40, 45, 50, 55, 60, 65, 70, 75, 80 or more percent cells.

In some embodiments, the compacting of the bio-ink results in a composition that is extrudable, allowing formation of multicellular aggregates or multicellular bodies. In some embodiments, “extrudable” means able to be shaped by forcing (e.g., under pressure) through a nozzle or orifice (e.g., one or more holes or tubes). In some embodiments, the compacting of the bio-ink results from growing the cells to a suitable density. The cell density necessary for the bio-ink will vary with the cells being used and the tissue or organ being produced.

In some embodiments, the cells of the bio-ink are cohered and/or adhered. In some embodiments, “cohere,” “cohered,” and “cohesion” refer to cell-cell adhesion properties that bind cells, multicellular aggregates, multicellular bodies, and/or layers thereof. In further embodiments, the terms are used interchangeably with “fuse,” “fused,” and “fusion.” In some embodiments, the bio-ink additionally comprises support material, cell culture medium (or supplements thereof), extracellular matrix (or components thereof), cell adhesion agents, cell death inhibitors, anti-apoptotic agents, anti-oxidants, extrusion compounds, and combinations thereof.

### Extrusion Compounds

In some embodiments, the bio-ink comprises an extrusion compound (i.e., a compound that modifies the extrusion properties of the bio-ink). Examples of extrusion compounds include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, gelatin, other biocompatible natural or synthetic polymers, nanofibers, and self-assembling nanofibers. In some embodiments, extrusion compounds are removed by physical, chemical, or enzymatic means subsequent to bioprinting, subsequent to cohesion of the bioprinted cells, or subsequent to maturation of the bioprinted construct.

Suitable hydrogels include those derived from collagen, hyaluronate, hyaluronan, fibrin, alginate, agarose, chitosan, and combinations thereof. In other embodiments, suitable hydrogels are synthetic polymers. In further embodiments, suitable hydrogels include those derived from poly(acrylic acid) and derivatives thereof, poly(ethylene oxide) and copolymers thereof, poly(vinyl alcohol), polyphosphazene, and combinations thereof. In various specific embodiments, the confinement material is selected from: hydrogel, NovoGel® (Organovo, Inc.; San Diego, Calif.), agarose, alginate, gelatin, Matrigel™, hyaluronan, poloxamer, peptide hydrogel, poly(isopropyl n-polyacrylamide), polyethylene glycol diacrylate (PEG-DA), hydroxyethyl methacrylate, polydimethylsiloxane, polyacrylamide, poly(lactic acid), silicon, silk, or combinations thereof.

In some embodiments, hydrogel-based extrusion compounds are crosslinkable gels. In further embodiments, crosslinkable gels include those crosslinkable by chemical means. For example, in some embodiments, suitable hydrogels include alginate-containing crosslinkable hydrogels. In various embodiments, suitable hydrogels comprise about 0.1, 0.5, 1, 2, 3, 4, 5, 6, 7, 8, 9, 10, or more percent alginate. In some embodiments, following bioprinting, constructs are optionally incubated with an agent to chemically crosslink the hydrogel, such as a solution of CaCl₂, in order preserve a bioprinted architecture prior to cohesion of the cells. Further, in some embodiments, the bioprinted constructs are optionally incubated with alginate lyase to enzymatically degrade the hydrogel. In further embodiments, the bioprinted constructs are optionally incubated with alginate lyase at a concentration of about 0.2-0.5 mg/ml to enzymatically degrade the hydrogel.

In some embodiments, suitable hydrogels include gelatin. In various embodiments, suitable hydrogels comprise about 1, 2, 3, 4, 5, 6, 7, 8, 9, 10, 11, 12, 13, 14, 15, 16, 17, 18, 19, 20 or more percent gelatin.

In some embodiments, the concentration of gelatin is about 5-15% and the concentration of alginate is about 0.5-5% in the extrusion compound or hydrogel. In a particular embodiment, the concentration of gelatin is 10% and the concentration of alginate is 1% in the extrusion compound or hydrogel.

In some embodiments, hydrogel-based extrusion compounds are thermoreversible gels (also known as thermo-responsive gels or thermogels). In some embodiments, a suitable thermoreversible hydrogel is not a liquid at room temperature. In specific embodiments, the gelation temperature (Tgel) of a suitable hydrogel is about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., or 40° C., including increments therein. In certain embodiments, the Tgel of a suitable hydrogel is about 10° C. to about 40° C. In further embodiments, the Tgel of a suitable hydrogel is about 20° C. to about 30° C. In some embodiments, the bio-ink (e.g., comprising hydrogel, one or more cell types, and other additives, etc.) described herein is not a liquid at room temperature. In some embodiments, a suitable thermoreversible hydrogel is not a liquid at mammalian body temperature. In specific embodiments, the gelation temperature (Tgel) of a suitable hydrogel is about 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 41° C., 43° C., 44° C., 45° C., 46° C., 47° C., 48° C., 49° C., 50° C., 51° C., or 52° C., including increments therein. In certain embodiments, the Tgel of a suitable hydrogel is about 22° C. to about 52° C. In further embodiments, the Tgel of a suitable hydrogel is about 32° C. to about 42° C. In some embodiments, the bio-ink (e.g., comprising hydrogel, one or more cell types, and other additives, etc.) described herein is not a liquid at mammalian body temperature. In specific embodiments, the gelation temperature (Tgel) of a bio-ink described herein is about 10° C., about 15° C., about 20° C., about 25° C., about 30° C., about 35° C., about 40° C., about 45° C., about 50° C., or about 55° C., including increments therein.

Polymers composed of polyoxypropylene and polyoxyethylene form thermoreversible gels when incorporated into aqueous solutions. These polymers have the ability to change from the liquid state to the gel state at temperatures maintainable in a bioprinter apparatus. The liquid state-to-gel state phase transition is dependent on the polymer concentration and the ingredients in the solution.

In some embodiments, the viscosity of the hydrogels and bio-inks presented herein is measured by any means described. For example, in some embodiments, an LVDV-II+CP Cone Plate Viscometer and a Cone Spindle CPE-40 are used to calculate the viscosity of the hydrogels and bio-inks. In other embodiments, a Brookfield (spindle and cup) viscometer is used to calculate the viscosity of the hydrogels and bio-inks. In some embodiments, the viscosity ranges referred to herein are measured at room temperature. In other embodiments, the viscosity ranges referred to herein are measured at body temperature (e.g., at the average body temperature of a healthy human).

In further embodiments, the hydrogels and/or bio-inks are characterized by having a viscosity of between about 500 and 1,000,000 centipoise, between about 750 and 1,000,000 centipoise; between about 1000 and 1,000,000 centipoise; between about 1000 and 400,000 centipoise; between about 2000 and 100,000 centipoise; between about 3000 and 50,000 centipoise; between about 4000 and 25,000 centipoise; between about 5000 and 20,000 centipoise; or between about 6000 and 15,000 centipoise.

In some embodiments, the non-cellular components of the bio-ink (e.g., extrusion compounds, etc.) are removed prior to use of the three-dimensional, engineered, biological cancer models. In further embodiments, the non-cellular components are, for example, hydrogels, peptide hydrogels, amino acid-based gels, surfactant polyols, thermo-responsive polymers, hyaluronates, alginates, collagens, or other biocompatible natural or synthetic polymers. In still further embodiments, the non-cellular components are removed by physical, chemical, or enzymatic means. In some embodiments, a proportion of the non-cellular components remain associated with the cellular components at the time of use.

### Pre-Formed Scaffold

In some embodiments, the three-dimensional, engineered, biological cancer models are free or substantially free of any pre-formed scaffold. In further embodiments, “scaffold” refers to synthetic scaffolds such as polymer scaffolds and porous hydrogels, non-synthetic scaffolds such as pre-formed extracellular matrix layers, dead cell layers, and decellularized tissues, and any other type of pre-formed scaffold that is integral to the physical structure of the engineered tissue and/or organ and not removed from the tissue and/or organ. In still further embodiments, decellularized tissue scaffolds include decellularized native tissues or decellularized cellular material generated by cultured cells in any manner; for example, cell layers that are allowed to die or are decellularized, leaving behind the ECM they produced while living.

In some embodiments, the three-dimensional, engineered, biological cancer models (including arrays of the same) do not utilize any pre-formed scaffold, e.g., for the formation of the tissue, any layer of the tissue, or formation of the tissue's shape. As a non-limiting example, the three-dimensional, engineered, biological cancer models may not utilize any pre-formed, synthetic scaffolds such as polymer scaffolds, pre-formed extracellular matrix layers, or any other type of pre-formed scaffold at the time of manufacture or at the time of use. In some embodiments, the three-dimensional, engineered, biological cancer models are substantially free of any pre-formed scaffolds. In further embodiments, the cellular components of the three-dimensional, engineered, biological cancer models contain a detectable, but trace or trivial amount of scaffold, e.g., less than 2.0%, less than 1.0%, less than 0.5%, or less than 0.1% of the total composition. In still further embodiments, trace or trivial amounts of scaffold are insufficient to affect long-term behavior of the three-dimensional, engineered, biological cancer models, or array thereof, or interfere with its primary biological function. In additional embodiments, scaffold components are removed post-printing, by physical, chemical, or enzymatic methods, yielding an engineered tissue that is free or substantially-free of scaffold components.

### Methods of Inserting Tumors, Tumor Fragments, Tumor Cells and Immortalized Cells

In some embodiments, the methods of fabricating the three-dimensional, engineered, biological cancer model comprises making an incision in the three-dimensional, engineered, tissue. In further embodiments, the incision forms an opening, cavity, or pocket in the three-dimensional, engineered, tissue. In various embodiments, the incision is suitably made by a cutting tool such as a scalpel, scissor, or the like. In another embodiment, the incision is suitably made by a laser. In various further embodiments, the incision is suitably made manually or with the aid of an automated or semi-automated robotic apparatus.

In some embodiments, the methods of fabricating three-dimensional, engineered, biological cancer models comprise engrafting cancer cells, tumor or tumor fragment(s) into the opening to form the three-dimensional, engineered, biological cancer tumor model. In various embodiments, the engrafting is suitably performed manually or with the aid of an automated or semi-automated robotic apparatus. In further embodiments, the cancer tumor or tumor fragment(s) is an undissociated, primary, patient-derived cancer tumor or tumor fragment(s). In one embodiment, the cancer tumor or tumor fragment is excised from a patient genetically non-identical to the donor source of one, some, or all of the stromal cell types of the stromal bio-ink.

In alternative embodiments, the primary cancer cells, tumor or tumor fragment(s) or immortalize cells are introduced into the three-dimensional, engineered, tissue to form the three-dimensional, engineered, biological cancer tumor model without first making an incision. By way of example, in one embodiment, the cancer tumor, tumor fragment(s), tumor cells or immortalized cells are introduced into the three-dimensional, engineered, tissue by injection, e.g., via a mechanically-controlled puncturing device. By way of further example, in other embodiments, the cancer tumor, tumor fragment(s), tumor cells or immortalized cells are introduced into the three-dimensional, engineered, tissue by manually pushing with a tool, shooting via a jet of compressed gas, or the like.

Many types of cancer cells, tumor, or tumor fragment(s) are suitably engrafted into an engineered stromal tissue to form a three-dimensional, engineered, biological tumor model. For example, many epithelial solid tumor types are suitable for use in the methodologies described herein, provided the cell(s), tumor, or tumor fragment(s) was surrounded by a relevant stromal tissue type. In one embodiment, suitable cancer cell, tumor, or tumor fragment(s) types include non-invasive ductal carcinoma, invasive ductal carcinoma, invasive lobular carcinoma, inflammatory breast cancer, paget disease of the nipple, phyllodes tumor, angiosarcoma, adenoid cystic carcinoma, low-grade adenosquamous carcinoma, medullary carcinoma, mucinous carcinoma, papillary carcinoma, tubular carcinoma, metaphasic carcinoma, micropapillary carcinoma and mixed carcinoma.

In one embodiment, the tumor(s), tumor fragment(s), cells or immortalized cells are breast cancer tumor(s), tumor fragment(s), cells or immortalized cells. Breast cancers are divided into categories based on different criteria. Most breast cancers are carcinomas. Other types of cancers can occur in the breast, too, such as sarcomas. Based on pathology, breast cancers can be categorized into ductal carcinoma in situ, invasive (or infiltrating) ductal carcinoma, invasive (or infiltrating) lobular carcinoma, inflammatory breast cancer, paget disease of the nipple, phyllodes tumor, and angiosarcoma. The sub-types of invasive carcinoma include adenoid cystic (or adenocystic) carcinoma, low-grade adenosquamous carcinoma, medullary carcinoma, mucinous (or colloid) carcinoma, papillary carcinoma, tubular carcinoma, metaplastic carcinoma (including spindle cell and squamous), micropapillary carcinoma, and mixed carcinoma (has features of both invasive ductal and lobular) (see the web at cancer.org/cancer/breastcancer/detailedguide/breast-cancer-breast-cancer-types). The receptor status of breast cancers may be identified by immunohistochemistry (IHC) based on the presence of estrogen receptors (ER), progesterone receptors (PR) and human epidermal growth factor receptor 2 (HER2). Additional breast cancer intrinsic subtypes based on genomic data include Luminal A, Luminal B, HER2-enriched (HER2E), Claudin-low, Basal-like, and a Normal Breast-like group. Luminal A tumors are often ER positive and HER2 negative. Luminal B tumors are often ER and PR positive. They can be HER2 positive or negative. Basal-like tumors are ER/PR/HER2 negative and also called triple negative breast cancer (TNBC). HER2E tumors are characterized by over-expressing genes in the HER2 amplicon such as GRB7 and PGAP3. They can be HER2 positive or negative. Claudin-low is a more recently described subtype. It is often triple-negative, has low expression of cell-cell junction proteins including E-cadherin, and frequently shows infiltration with lymphocytes. Genetic markers such as TP53, PIK3CA, GATA3 PTEN, AKT1, CDH1, RB1, MLL3, MAP3K1, CDKN1B, TBX3, RUNX1, CBFB, AFF2, PIK3R1, PTPN22, PTPRD, NF1, SF3B1, CCND3, and TBX3, etc., have been associated with breast cancers. In addition, certain genetic markers are found to be enriched in different subtypes. For example, A HER2/p-HER2/HER1/p-HER1 signature is identified within the HER2-Enriched subtype. Genetic markers for HER2E/HER2-positive tumors include high expression of including FGFR4, HER1/EGFR, HER2 itself, and genes within the HER2-amplicon (i.e. GRB7). Genetic markers for Luminal/ER-positive breast cancers include high expression of ESR1, BCL2, GATA3, FOXA1, XBP1 and cMYB, frequent mutations in MAP3K1, MAP2K4, PIK3CA, GATA3, FOXA1, ESR1, XBP1, RUNX1, and CBFB, loss of ATM, PTEN, and INPP4B, TP53 inactivation, amplification of MDM2, FGFRs, IGFR1, and Cyclin D1/CDK4/CDK6. Genetic markers for Basal-like tumors (TNBC) include BRCA1 inactivation, amplification of Cyclin E1, PIK3CA, KRAS, BRAF, HER1/EGFR, cMYC, FGFR1, FGFR2, IGFR1, c-KIT, MET, PDGFRA, and FOXM1, a high frequency of mutations in TP53 and PIK3CA, high PI3K/AKT and HIF1a/ARNT pathway activities, loss of RB1, PTEN, and INPP4B, and high expression of Keratins 5, 6 and 17 and genes associated with cell proliferation. Genetic markers for Luminal A subtype include the enrichment of specific mutations in MAP3K1, MAP2K4, GATA3, PIK3CA, TP53, CDH1, RUNX1, and CBFB, high expression of ESR1, GATA3, FOXA1, XBP1 cMYB, ER, PR, AR, BCL2, GATA3, and INPP4B. Genetic markers that have low expression in Claudin-low subtype tumor (mostly TNBC) include HER2, ESR1, GATA3, the luminal keratins 8 and 18, claudin 3, 4, and 7, cingulin, occludin, E-cadherin, CD24, EpCAM and MUC1. Genetic markers that have high expression in Claudin-low subtype tumor include genes expressed by T- and B-lymphoid cells (i.e. CD4 and CD79a), interleukin 6, CXCL2, vimentin, N-cadherin, and several known transcriptional repressors of E-cadherin (i.e. TWIST1). In addition, breast cancers can be characterized by EGFR or KRAS status. For example, 30% of IBC cases, the most clinically aggressive subtype of breast cancer, and at least 50% of cases of TNBC are also associated with EGFR overexpression. KRAS mutant is significantly enriched in HER2 overexpressing breast cancers. Genome wide genetic marker analyses can be done by various methods, such as genomic DNA copy number arrays, DNA methylation, exome sequencing, mRNA arrays, microRNA sequencing and reverse phase protein arrays. The comprehensive genetic profiles of breast cancers and subtypes and methods for detecting are described by Dai X, Li T, Bai Z, Yang Y, Liu X, Zhan J, Shi B (2015). Breast cancer intrinsic subtype classification, clinical use and future trends. Am J Cancer Res. 5(10):2929-43; Prat A and Perou C M (2011). Deconstructing the molecular portraits of breast cancer. Mol Oncol. 5(1):5-23; and Koboldt D C (2012). Comprehensive molecular portraits of human breast tumours. Nature. 2012 490(7418):61-70; and Masuda H, Zhang D, Bartholomeusz C, Doihara H, Hortobagyi G N, and Ueno N T (2012). Role of epidermal growth factor receptor in breast cancer. Breast Cancer Res Treat. 136(2):331-45.

In one embodiment, the breast cancer tumor(s), tumor fragment(s), tumor cells or immortalized cells are obtained from a human patient. Breast cancer cell lines are commercially available from, for example, ATCC or Sigma. For instance, the ATCC Breast Cancer Biomarkers Cell Line Panel 1 (ATCC® TCP-1004™) includes a panel of breast cancer cells with distinct pathological subtypes and genetic marker information. In addition, a list of breast cancer cell lines with corresponding subtype genetic profile is provided in Holliday D L and Speirs V (2011). Choosing the right cell line for breast cancer research. Breast Cancer Res. 2011 13(4):215. The genetic mutation information of commonly used cancer cell lines is available from Sanger COSMIC cell line database (cancer.sanger.ac.uk/cell_lines/cbrowse/all). In addition, cancer cell lines characterized by genetic mutations are available from ATCC. Breast cancer tumors are commercially available from, for example, US Biomax Inc. (biomax.us/tissue-section.php?product=HuFTU), CureLine (cureline.com/human-tumor-tissues), or Discovery Life Sciences (discoverylifesciences.com/clinical-research-specimens/cancer). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are intestinal cancer tumor(s), tumor fragment(s), cells or immortalized cells. Colorectal cancers are categorized into four consensus molecular subtypes (CMS) by the Colorectal Cancer Subtyping Consortium by combining genomic datasets from gene expression profiles. CMS1 includes 14% of the patients and is characterized by CpG island methylator phenotype (CIMP)-high, hypermutation, BRAF mutations, increased expression of genes related to diffuse immune infiltration consisting mainly of TH1 and cytotoxic T-cells, activation of immune evasion pathway, and low somatic copy number alterations (SCNAs). CMS1 includes most microsatellite instability (MSI) carcinomas with overexpression of DNA damage-repair proteins and impaired DNA mismatch repair ability. CMS2 is the most common subset comprising 37% of the patients. CMS2 tumors display chromosomal instability, strong WNT/MYC pathway activation, TP53 mutation, and EGFR amplification/overexpression. CMS3 tumors are seen in 13% of the patients. They are associated with low chromosomal instability, moderate WNT/MYC pathway activation, KRAS and PIK3CA mutation, and IGFBP2 overexpression. CMS4 comprises 23% of the patients. CMS4 tumors are CIN/MSI heterogeneous and characterized with mesenchymal/TGF-β activation and NOTCH3/VEGFR2 overexpression.

The genetic markers for colorectal cancer include genes involved in the mismatch repair pathway, such as MLH1, MSH2, MSH3, MSH6, PMS2, POLE, and POLD1, etc. Additional genetic markers for colorectal cancer include BRAF, PIK3CA, P53, RAS, H3K36me3, PTEN, MET, EGFR, ALK/ROS1, and ATM. The genetic markers associated with colorectal cancer can be identified by, for example, gene expression profiling. The subtypes of colorectal cancer, genetic markers, and methods of detection are reviewed by Guinney J, et al. (2015). The consensus molecular subtypes of colorectal cancer. Nat Med. 21(11):1350-1356; Graham D M, Coyle V M, Kennedy R D, Wilson R H (2016). Molecular Subtypes and Personalized Therapy in Metastatic Colorectal Cancer. Curr Colorectal Cancer Rep. 12:141-150; and Yiu A J and Yiu C Y (2016). Molecular markers in Colorectal Cancer. Anticancer Res. 36(3):1093-102.

In one embodiment, the intestinal tumor(s), tumor fragment(s), tumor cells or immortalized cells are obtained from a human patient. Colorectal cancer cell lines are commercially available from, for example, ATCC or Sigma. The genetic mutation information of commonly used cancer cell lines is available from Sanger COSMIC cell line database. In addition, epigenetic and genetic features of 24 colon cancer cell lines and sources of such cell lines are provided in Ahmed D, Eide P W, Eilertsen I A, Danielsen S A, Eknxs M, Hektoen M, Lind G E, and Lothe R A (2013). Epigenetic and genetic features of 24 colon cancer cell lines. Oncogenesis. 2:e71. In addition, a comprehensive list of colorectal cancer cell lines and genetic information can be found at Colorectal Cancer Atlas (see the web at colonatlas.org/browse). Colorectal cancer tumors are commercially available from, for example, US Biomax Inc. (biomax.us/tissue-section.php?product=HuFTU) or CureLine (cureline.com/human-tumor-tissues). In addition, a comprehensive directory of biobanks, tissue banks, and biorepositories can be accessed at specimencentral.com/biobank-directory. Further, fresh frozen tumors, including custom tumor collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are lung cancer tumor(s), tumor fragment(s), cells or immortalized cells. Lung cancers are commonly categorized into three main types: Non-Small Cell Lung Cancer (NSCLC), small cell lung cancer, and lung carcinoid tumor. NSCLC further includes subtypes of squamous cell carcinoma, adenocarcinoma, and large cell carcinoma (see the web at cancer.org). Exemplary genetic markers for lung cancer include iNTR, TUBB3, RRM1, ERCC1, BRCA1, p53, BCL-2, ALK, MRP2, MSH2, TS, mucin, BAG-1, pERK1/2, pAkt-1, p2′7, TUBB3, PARP1, ATM, and TopIIA. Additional genetic markers for lung cancer include CCND1, Napsin-A, and TTF-1. In addition, genetic markers associated with squamous cell carcinoma include: CDKN2A, TP53, PTEN, PIK3CA, KEAP1, MLL2, HLA-A, NFE2L2, NOTCH1/2, RB1, FAM123B (WTX), HRAS, FBXW7, SMARCA4, NF1, SMAD4, EGFR, APC, TSC1, BRAF, TNFAIP3, CREBBP, NFE2L2, KEAP1, CUL3, FGFR1, WHSC1L1, SOX2, TP63, ASCL4, and FOXP1. The molecular and genetic markers for lung cancer can be assessed by IHC, RNA-Seq, and gene expression microarray. Comprehensive reviews of such markers and methods for detection are provided in Wallerek S and Sorensen J B (2015). Biomarkers for efficacy of adjuvant chemotherapy following complete resection in NSCLC stages I-IIIA. Eur Respir Rev. 24(136):340-55; Zhu C Q and Tsao M S (2014). Prognostic markers in lung cancer: is it ready for prime time? Transl Lung Cancer Res. 3(3):149-58; Ye J, Findeis-Hosey J J, Yang Q, McMahon L A, Yao J L, Li F, Xu H (2011). Combination of napsin A and TTF-1 immunohistochemistry helps in differentiating primary lung adenocarcinoma from metastatic carcinoma in the lung. Appl Immunohistochem Mol Morphol. 19(4):313-7; Hammerman P S et al. (2012) Comprehensive genomic characterization of squamous cell lung cancers. Nature. 489(7417):519-25.

In one embodiment, the lung cancer tumor(s), tumor fragment(s), tumor cells or immortalized cells are obtained from a human patient. Lung cancer cell lines and genetic information of the cell lines are available from commercial sources, such as ATCC or Sigma. National Cancer Institute provides a panel of 60 human cancer cell lines (commonly called the NCI-60 cell lines), including lung cancer. The genetic mutation information of commonly used cancer cell lines is available from Sanger COSMIC cell line database. The list can be accessed on the web at dtp.cancer.gov/discovery_development/nci-60/cell_list.htm. In addition, 17 genetic markers including: AKT1, BRAF, MET, EGFR, ERBB2, KRAS, STK11, MYC, MYCL, MYCN, NRAS, PIK3CA, PTEN, CDKN2A, RB1, TP53, and the EML4-ALK fusion, have been tested in 18 commonly used lung cancer cell lines. The information is provided in Blanco R, Iwakawa R, Tang M, Kohno T, Angulo B, Pio R, Montuenga L M, Minna J D, Yokota J, and Sanchez-Cespedes M (2009). A gene-alteration profile of human lung cancer cell lines. Hum Mutat. 30(8):1199-206. Further, lung cancer tumors are commercially available from, for example, US Biomax Inc. (biomax.us/tissue-section.php?product=HuFTU), CureLine (cureline.com/human-tumor-tissues), or Discovery Life Sciences, Inc. (pre-treatment: discoverylifesciences.com/clinical-research-specimens/cancer/lung-cancer-pre-treatment and NSCLC discoverylifesciences.com/clinical-research-specimens/cancer/nscic). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are gastric cancer tumor(s), tumor fragment(s), cells or immortalized cells. Gastric cancer can be categorized into four molecular subtypes based on gene expression data, including the mesenchymal-like type, microsatellite-unstable type, the tumor protein 53 (TP53)-active type, and TP53-inactive type. The genetic markers for gastric cancer can include micro RNAs, such as miR-1, miR-20a, miR-27a, miR-34, miR-196a, miR-378, miR-221, miR376c, miR-423-5p, let-7a, miR-17-5p, miR-21, miR-106a/b, miR-199a-3p, miR-218, miR-223, miR-370, miR-451, miR-486, miR-21, miR-106a, miR-129, and miR-421. Additional genetic markers for gastric cancer include TP53, PTKs (such as TIE-1 and MKK4), FYN, PLK1, GISP/RegIV, EGFR, ERBB2, VEGF, TGF, c-MET, IL-6, IL-11, Cyclin E, Bcl-2, Fas, survivin, Runx3, E-cadherin, WNTSA, IL-1, and IL-10. Other genetic markers for gastric cancer include carcinoembryonic antigen (CEA); alpha-fetoprotein (AFP); CA 19-9, CA 72-4, free beta-subunit of human choriogonadotropin (B-HCG), and pepsinogen I/II. The genetic markers for gastric cancer can be assessed by Northern blot analysis, immunological detection based on monoclonal/polyclonal antibodies, or gene expression microarray. In addition, these markers can be also assessed by restriction analysis of gene expression (RAGE) analysis or serial analysis of gene expression (SAGE). A more complete list of molecular markers for gastric cancer and methods for detection thereof are described in Cristescu R. et al., (2015). Molecular analysis of gastric cancer identifies subtypes associated with distinct clinical outcomes. Nat Med. 21(5):449-56; Hua-Hsi Wu, Wen-chang Lin, and Kuo-Wang Tsai (2014). Advances in molecular biomarkers for gastric cancer: miRNAs as emerging novel cancer markers. Expert Rev Mol Med. 16:e1; and Ziliang Jin, Weihua Jiang, and Liwei Wang (2015). Biomarkers for gastric cancer: Progression in early diagnosis and prognosis. Oncol Lett. 9(4): 1502-1508.

In one embodiment, the gastric tumor(s), tumor fragment(s), tumor cells or immortalized cells are obtained from a human patient. Gastric cancer cell lines are commercially available. For example, the Stomach (Gastric) Cancer Panel (ATCC® No. TCP-1008™) comprises 6 stomach cancer cell lines isolated from both primary and metastatic sites with identified genetic markers. Additional gastrointestinal tumor cell lines are also available from ATCC (see the web at atcc.org/˜/media/PDFs/Gastrointestinal_Tumor_Cell_Lines.ashx). The genetic mutation information of commonly used cancer cell lines is available from Sanger COSMIC database. In addition, gastric cancer tumors are commercially available from, for example, Discovery Life Sciences, Inc. (discoverylifesciences.com/clinical-research-specimens/cancer/gastric-cancer). Further, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are prostate cancer tumor(s), tumor fragment(s), cells or immortalized cells. Prostate cancers are categorized into seven distinct subtypes by The Cancer Genome Atlas (TCGA) Research Network based on specific gene fusions (ERG, ETV1, ETV4, and FLI1) or mutations (SPOP, FOXA1, and IDH1). Exemplary genetic markers for prostate cancer include but are not limited to NKX3.1, MYC, TMPRSS2-ERG translocations, PTEN, Akt/mTOR, Erk (p42/44), Her2/Neu or SRC tyrosine kinases, WNT, APC, k-RAS, β-catenin, FGFR1, FGF10, EZH2, PCA3, and AR. The genetic markers for prostate cancer can be assessed by, for example, gene expression profiling, miRNA expression profiling, serum proteomics, metabolomics, and whole exome sequencing. Such genetic markers and methods for detection thereof are provided in Abeshouse A, et al., (2015). The Molecular Taxonomy of Primary Prostate Cancer. Cell 163(4):1011-25 and Michael M. Shen and Cory Abate-Shen (2010) Molecular genetics of prostate cancer: new prospects for old challenges Genes & Dev. 24: 1967-2000.

In one embodiment, the prostate tumor(s), tumor fragment(s), tumor cells or immortalized cells are obtained from a human patient. Prostate cancer cell lines are available from, for example, the NCI-60 cell lines and ATCC. The genetic mutation information of commonly used cancer cell lines is available from COSMIC cell line database. For example, LNCaP clone FGC is available from ATCC (ATCC® CRL-1740™), and a detailed genetic profile is available from the COSMIC cell line database. In addition, genetic and molecular markers for prostate cancer cell lines are described in P. J. Russell and E. A. Kingsley. Ch. 2 Human Prostate Cancer Cell Lines, Methods in Molecular Medicine, Vol. 81: Prostate Cancer Methods and Protocols. Human prostate cancer tissue samples are commercially available, for example, from US Biomax Inc. (biomax.us/tissue-section.php?product=HuFTU), CureLine (cureline.com/human-tumor-tissues), or Discovery Life Sciences (pre-treatment: discoverylifesciences.com/clinical-research-specimens/cancer/prostate-cancer and discoverylifesciences.com/clinical-research-specimens/cancer). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are kidney cancer tumor(s), tumor fragment(s), cells or immortalized cells. Cancers of kidney include two main types: renal cell carcinoma (RCC) and transitional cell carcinoma (TCC). Renal cell carcinoma can be further divided into the following subtypes: clear cell (conventional) RCC, papillary RCC, chromophobe RCC, renal oncocytoma RCC, unclassified RCC, collecting duct carcinoma, medullary RCC, and sarcomatoid RCC (see the web at kidneycancer.org/knowledge/learn/about-kidney-cancer/). Exemplary molecular markers for RCC include neuron-specific enolase (NSE), TRAF-1, Hsp27, IL-1, IL-6, TNF-α, serum amyloid A (SAA), C-reactive protein (CRP), gamma-glutamyl transferase (GGT), tumor necrosis factor-related apoptosis-inducing ligand (TRAIL), cytokeratins (CK), serum M65 (the intact form of cytokeratin 18), hypoxia-inducible transcriptional factors (HIF-1α and HIF-1β), VEGF, Von Hippel-Lindau (VHL), prolyl hydroxylase-3 (PHD3), pyruvate kinase isoenzyme type M2 (TuM2-PK), thymidine kinase 1 (TK1), 20S proteasome, Fetuin A, Osteopontin (OPN), Osteoprotegerin, NMP-22, NGAL, KIM-1, MMPs, and PLIN2. Molecular markers for kidney cancers can be assessed by immunoassays, such as ELISA or immunonephelometry, or mass spectrometry. Kusama K, Asano M, Moro I, Sekine T, Kakizoe T, Tobisu K, and Kishi K (1991). Tumor markers in human renal cell carcinoma. Tumour Biol. 12(4):189-97 and Pastore A L, Palleschi G, Silvestri L, Moschese D, Ricci S, Petrozza V, Carbone A, Di Carlo A (2015). Serum and urine biomarkers for human renal cell carcinoma. Dis Markers. 2015:251403.

In one embodiment, the kidney tumor(s), tumor fragment(s), tumor cells or immortalized cells are obtained from a human patient. Kidney cancer cell lines are commercially available, e.g., from ATCC or Sigma, and the genetic information of the cell lines can be found at COSMIC cell line database. Kidney cancer tumors can be obtained commercially, for example, from US Biomax Inc. (biomax.us/tissue-section.php?product=HuFTU) or CureLine (cureline.com/human-tumor-tissue). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).Skin cancer includes actinic keratosis, basal cell carcinoma, melanoma, kaposi's sarcoma (KS), merkel cell carcinoma, and squamous cell carcinoma (see the web at skincancer.org/skin-cancer-information and dermatology.ucsf.edu/skincancer/professionals/types.aspx). In addition, melanoma can be divided into four subtypes based on the pattern of the most prevalent significantly mutated genes: mutant BRAF, mutant RAS, mutant NF1, and Triple-WT (wild type). Exemplary genetic markers for skin cancer (such as melanoma) include, in addition to the above, NRAS, CDKN2A/B, TP53, PTEN, RAC1, MAP2K1, PPP6C, ARID2, F1, IDH1, RB1, DDX3X, RAC1, IDH1, MRPS31, RPS27, TERT, phospho-MAP2K1/MAP2K2 (MEK1/2), MAPK1/MAPK3 (ERK1/2), CDK4, and CCND1. The genetic markers for skin cancer (such as melanoma) can be assessed by DNA copy-number and single-nucleotide polymorphism array, whole-genome sequencing, RNA-Seq, reverse-phase protein array, and microRNA sequencing. A more complete list of genetic markers for skin cancer and methods for detection thereof are provided in Akbani R. et al. (2015). Genomic Classification of Cutaneous Melanoma. Cell. 161(7):1681-96.

In one embodiment, the tumor(s), tumor fragment(s), cells or immortalized cells are skin cancer tumor(s), tumor fragment(s), cells or immortalized cells. In one embodiment, the skin cancer tumor(s), tumor fragment(s), tumor cells or immortalized cells are obtained from a human patient. Skin cancer cell lines are commercially available from, for example, ATCC or Sigma. In particular, the ATCC Metastatic Melanoma Cancer Cell Panel (ATCC® TCP-1014™) comprises 4 metastatic melanoma cancer cell lines with varying degrees of genetic complexity. Genetic mutations in one or more of the following genes: BRAF, TP53, CDKN2A and PTEN, have been identified according to COSMIC cell line data base. Skin cancer tumors are commercially available, for example, from US Biomax Inc. (biomax.us/tissue-section.php?product=HuFTU) or CureLine (melanoma, including B-raf status, cureline.com/human-tumor-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are ovarian cancer tumor(s), tumor fragment(s), cells or immortalized cells. Ovarian cancers can be divided into more than 30 types on the basis of the type of cell from which they start, such as epithelial ovarian cancers, ovarian germ cell cancers, and ovarian stromal cancers, etc. Epithelial ovarian cancers further comprise a heterogeneous group of neoplasms including the four most common subtypes: serous, endometrioid, clear cell, and mucinous. About 10% of the epithelial ovarian cancers are undifferentiated or unclassifiable. Common genetic markers of ovarian cancer include B-RAF, K-RAS, TP53, and BRCA1/2. Additional genetic markers associated with ovarian cancer include CA125, CA 19.9, CA 15.3, TAG.72, MSH2, MLH1, MLH6, PMS1, PMS2, ESR2, BRIP1, MSH6, RAD51C, RAD51D, CDH1, CHEK2, PALB2, RAD50, OVX1, sFas, CYFRA 21.1, VEGF, and human kallikrein 10 (hK10). In addition, molecular markers associated with ovarian germ cell tumors include Alpha-fetoprotein (αFP), M-CSF, and LDH, etc. Further, exemplary molecular markers associated with ovarian granulosa cell tumor (a rare type of ovarian cancer and a sub-type of stromal cancer) include inhibin α, betaA, and betaB subunits. Such genetic markers can be assessed by, for example, direct sequencing or microarray analysis. Additional detection method includes immunostaining. McCluggage W G (2011). Morphological subtypes of ovarian carcinoma: a review with emphasis on new developments and pathogenesis. Pathology. 43(5):420-32; Norquist B M et al. (2015). Inherited mutations in women with ovarian carcinoma. JAMA Oncol. 30: 1-9; Kuusisto K M, Bebel A, Vihinen M, Schleutker J, Sallinen S L (2011). Screening for BRCA1, BRCA2, CHEK2, PALB2, BRIP1, RAD50, and CDH1 mutations in high-risk Finnish BRCA1/2-founder mutation-negative breast and/or ovarian cancer individuals. Breast Cancer Res. 13 (1): R20; and Gadducci A, Cosio S, Carpi A, Nicolini A, and Genazzani A R (2004). Serum tumor markers in the management of ovarian, endometrial and cervical cancer. Biomed Pharmacother. 58(1):24-38.

In one embodiment, the ovarian tumor(s), tumor fragment(s), tumor cells or immortalized cells are obtained from a human patient. Ovarian cancer cell lines are commercially available from, for example, ATCC or Sigma. For instance, the Ovarian Cancer Panel (ATCC® No. TCP-1021™) includes four ovarian cancer cell lines, which have genomic mutations in one or more of the following genes: APC, CDKN2A, FAM123B, KRAS, MLH1, NRAS, PIK3CA, STK11, and TP53, according to the COSMIC cell line database. Ovarian tumor tumors are commercially available, for example, from Discovery Life Sciences (discoverylifesciences.com/clinical-research-specimens/cancer/ovarian-carcinoma) CureLink (cureline.com/human-tumor-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are cervical cancer tumor(s), tumor fragment(s), cells or immortalized cells. The two main types of cervical cancer are squamous cell carcinoma and adenocarcinoma. Serum levels of CA 19-9, CA 125, CA 15-3, SCC antigen, CYFRA 21.1, CEA, M-CSF, sFas, VEGF, and thymidine kinase (TK) are commonly used molecular markers for cervical cancer. Genetic markers associated with cervical cancers include p16ink4a, MCM 3 and 5, CDC6, Geminin, Cyclins A-D, TOPO2A, CDCA1, and BIRC5. Other genetic markers for cervical cancer include UBE2C, CCNB1, CCNB2, PLOD2, NUP210, MELK, CDC20, IL8, INDO, ISG15, ISG20, AGRN, DTXL, MMP1, MMP3, CCL18, and STAT1. In addition, molecular markers that are overexpressed in squamous cell cervical cancer include ribosomal protein S12, the mitochondrial subunit NADH dehydrogenase 4, 16S ribosomal RNA (rRNA), and capping protein muscle Z-line al, etc. Such genetic markers can be detected by high density microarrays and the expression of mRNA and protein can be validated by real-time PCR and IHC, respectively. Additional detection methods include RNA-RNA in situ hybridization, Northern blot analysis, Reverse Northern blot, and Differential display and gene fragments cloning, etc. A comprehensive list of genetic markers associated with cervical cancer and methods for detection are provided in Gadducci A, Cosio S, Carpi A, Nicolini A, and Genazzani A R (2004). Serum tumor markers in the management of ovarian, endometrial and cervical cancer. Biomed Pharmacother. 58(1):24-38; Martin C M, Astbury K, McEvoy L, O'Toole S, Sheils O, and O'Leary J J (2009). Gene expression profiling in cervical cancer: identification of novel markers for disease diagnosis and therapy. Methods Mol Biol. 511:333-59; Rajkumar T, Sabitha K, Vijayalakshmi N, Shirley S, Bose M V, Gopal G, and Selvaluxmy G (2011). Identification and validation of genes involved in cervical tumourigenesis. BMC Cancer. 11:80; and Cheng Q, Lau W M, Chew S H, Ho T H, Tay S K, Hui K M (2002). Identification of molecular markers for the early detection of human squamous cell carcinoma of the uterine cervix. Br J Cancer. 86(2):274-81.

In one embodiment, the cervical cancer tumor(s), tumor fragment(s), tumor cells or immortalized cells are obtained from a human patient. Cervical cancer cell lines are commercially available from ATCC or Sigma. For example, the ATCC Cervical Cancer Cell Panel (ATCC® TCP-1022™) comprises four cervical cancer cell lines of which the genetic mutation information is available from COSMIC cell line database. In addition, cervical cancer tumors are commercially available, for example, from BioreclamationlVT (bioreclamationivt.com/disease-state-tissues). A comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are uterine cancer tumor(s), tumor fragment(s), cells or immortalized cells. Uterine (endometrial) cancers can be divided into four subtypes: endometrioid adenocarcinoma, serous adenocarcinoma, adenosquamous carcinoma, and carcinomasarcoma (see the web at mskcc.org/cancer-care/types/uterine-endometrial). Of hereditary uterine cancers, approximately 50 to 70 percent are associated with the hereditary nonpolyposis colorectal cancer (HNPCC) syndrome, also known as Lynch syndrome (see the web at cedars-sinai.edu/Patients/Programs-and-Services/Womens-Cancer-Program/Patient-Guide/Uterine-cancer-genetic-risk.aspx). Genetic markers associated with Lynch Syndrome are also associated with hereditary uterine cancers. These genetic markers include MLH1, MSH2, MSH6, PMS2, EPCAM, PTEN, BRCA1, BRCA2, TP53, MUTYH, CDKN2A, PGR, and CHEK2. Such genetic markers are commonly identified by genome-wide association study. Meyer L A, Broaddus R R, Lu K H (2009). Endometrial cancer and Lynch syndrome: clinical and pathologic considerations. Cancer Control. 16(1):14-22 and Gayther S A and Pharoah P D (2010). The inherited genetics of ovarian and endometrial cancer. Curr Opin Genet Dev 20(3):231-8.

In one embodiment, the uterine cancer tumor(s), tumor fragment(s), tumor cells or immortalized cells are obtained from a human patient. Uterine cancer cell lines can be obtained from commercial sources, e.g. ATCC. For example, HEC-1-A (ATCC® HTB-112™) is an endometrial adenosquamous carcinoma of which the genetic mutation information for HEC-1-A cell line is available from COSMIC cell line database. Uterine cancer tumors are commercially available, for example, from BioreclamationlVT (bioreclamationivt.com/disease-state-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are liver cancer tumor(s), tumor fragment(s), cells or immortalized cells. Primary liver cancers include four main types: hepatocellular carcinoma (HCC), cholangiocarcinoma (bile duct cancer), angiosarcoma (also called haemangiosarcoma), and hepatoblastoma (see the web at cancerresearchuk.org/about-cancer/liver-cancer/types). AFP (including AFP-L1, AFP-L2 and AFP-L3) is the most widely used tumor biomarker currently available for the early detection of HCC. Additional molecular and genetic markers for HCC include HSP70, HSP27, Glypican-3 (GPC3), squamous cell carcinoma antigen (SCCA), Golgi protein 73 (GP73, also known as Golph2 and GOLM1), Tumor-associated glycoprotein 72 (TAG-72), Zinc-a2-glycoprotein (ZAG), Des-γ-carboxyprothrombin (DCP), γ-glutamyl transferase (GGT), α-1-fucosidase (AFU), Transforming growth factor-β1 (TGF-β1), and VEGF, microRNAs, such as miR-500, miR-122, miR-29, and miR-21, Δ-like 1 homolog (DLK1), and Villin1 (Vil1). Additional genetic markers for HCC include TP53, CD34, RGS5, THY1, ADAMTS1, MMP2, MMP14, keratin 17, keratin 19, and mucin 1, etc. AFP can be detected by various methods, such as isoelectric focusing (IEF), lectin-electrophoresis, or immunoassays including RIA, IRMA, MEIA, nephelometry, and electrochemiluminescence, etc. Other detection methods used for identifying the molecular and genetic markers associated with HCC include IHC, Southern blot, and cDNA microarray, etc. The molecular and genetic markers for liver cancer and methods for detection are disclosed in Debruyne E N and Delanghe J R (2008). Diagnosing and monitoring hepatocellular carcinoma with alpha-fetoprotein: new aspects and applications. Clin Chim Acta. 395(1-2):19-26; Zhao Y J, Ju Q, and Li G C (2013). Tumor markers for hepatocellular carcinoma. Mol Clin Oncol1(4):593-598; and Chen X, Cheung S T, So S, Fan S T, Barry C, Higgins J, Lai K M, Ji J, Dudoit S, Ng I O, Van De Rijn M, Botstein D, and Brown P O (2002). Gene expression patterns in human liver cancers. Mol Biol Cell. 13(6):1929-39.

In one embodiment, the liver cancer tumor(s), tumor fragment(s), tumor cells or immortalized cells are obtained from a human patient. Liver cancer cell lines can be obtained from commercial sources such as ATCC or Sigma. For example, the ATCC Liver Cancer Panel (ATCC® TCP-1011™) comprises 7 cell lines of which the genetic mutation information is available from COSMIC cell line database. Liver cancer tumors are commercially available, for example, from BioreclamationIVT (bioreclamationivt.com/disease-state-tissues) or Discover Life Sciences (discoverylifesciences.com/clinical-research-specimens/cancer/liver-cancer-pre%E2%80%93treatment). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are bladder cancer tumor(s), tumor fragment(s), cells or immortalized cells. Bladder cancers are divided into three main types based on histopathological observation: urothelial carcinoma (such as transitional cell carcinoma (TCC)), squamous cell carcinoma (SCC), and adenocarcinoma. Less common types of bladder cancer include sarcoma and small cell anaplastic cancer. In addition to its cell type, bladder cancer may be described as noninvasive, non-muscle-invasive, or muscle-invasive (see the web at cancer.net/cancer-types/bladder-cancer/overview). Genetic markers associated with bladder cancer include: HRAS, NRAS, KRAS2, FGFR3, ERBB2, CCND1, MDM2, E2F3, RASSF1A, FHIT, CDKN2A, PTCH, DBC1, TSC1, PTEN, RB1, and TP53. In addition, SULF1 and the lysosomal cysteine proteinases cathepsins B, K, and L, RGS1, RGS2, THBS1, THBS2, VEGFC, NRP2 are associated with muscle-invasive tumors. CTSE showed higher expression levels in superficial tumors. Other genetic markers associated with bladder cancers are MMP2, CCNA2, CDC2, CDC6, TOP2A, SKALP PRKAG1, GAMT, ACOX1, ASAH1, SCD, AF1Q, AREG, DUSP6, LYAR, MAL, and RARRES. Such genetic markers can be assessed by, for example, expression microarray analysis. Margaret A. Knowles (2006). Molecular subtypes of bladder cancer: Jekyll and Hyde or chalk and cheese? Carcinogenesis 27(3): 361-373; Knowles M A (2008). Bladder cancer subtypes defined by genomic alterations. Scand J Urol Nephrol Suppl. (218):116-30; and Blaveri E, Simko J P, Korkola J E, Brewer J L, Baehner F, Mehta K, Devries S, Koppie T, Pejavar S, Carroll P, Waldman F M (2005). Bladder cancer outcome and subtype classification by gene expression. Clin Cancer Res. 11(11):4044-55.

In one embodiment, the bladder cancer tumor(s), tumor fragment(s), tumor cells or immortalized cells are obtained from a human patient. Bladder cancer cell lines are commercially available, for example, from ATCC or Sigma. The genetic mutation information of most commonly used cancer cell lines is available from COSMIC cell line database. In addition, a comprehensive genomic characterization of 40 urothelial bladder carcinoma cell lines including information on mutation status of genes implicated in bladder cancer (FGFR3, PIK3CA, TP53, and RAS) is provided in Earl J., et al., (2015). The UBC-40 Urothelial Bladder Cancer cell line index: a genomic resource for functional studies. BMC Genomics. 16:403. Bladder cancer tumors are commercially available, for example, from US Biomax Inc. (biomax.us/tissue-section.php?product=HuFTU) or BioreclamationIVT (bioreclamationivt.com/disease-state-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are esophageal cancer tumor(s), tumor fragment(s), cells or immortalized cells. Esophageal cancer is a highly heterogeneous cancer dominated by copy number alterations and large-scale rearrangements. Esophageal cancer has two main types, i.e., squamous-cell carcinoma and adenocarcinoma. In addition, based on mutational signatures, esophageal cancer can be divided into three distinct molecular subtypes with potential therapeutic relevance, namely, DDR (DNA Damage Repair) impaired, C>A/T dominant, and mutagenic. Exemplary genetic markers associated with esophageal cancer include SMYD3, RUNX1, CTNNA3, RBFOX1, CDKN2A/2B, CDK14, ERBB2, EGFR, RB1, GATA4/6, CCND1, and MDM2, etc. The genetic markers associated with the DDR impaired subtype include TP53, ARID1A, and SMARCA4. Such genome wide genetic analysis can be done by deep sequencing, which is described in Secrier M, et al. (2016). Mutational signatures in esophageal adenocarcinoma define etiologically distinct subgroups with therapeutic relevance. Nat Genet. 48(10):1131-41.

In one embodiment, the esophageal cancer tumor(s), tumor fragment(s), tumor cells or immortalized cells are obtained from a human patient. Esophageal cancer cell lines, together with their genotyping information have been deposited with ECACC General Cell Collection. Additional esophageal cancer cell line is also available from commercial sources such as Sigma (e.g., 0E33 cell line). The genetic mutation information of esophageal cancer cell lines, for example, 0E33, is available from COSMIC cell line database. Esophageal cancer tumors are commercially available, for example, from US Biomax Inc. (biomax.us/tissue-section.php?product=HuFTU) or BioreclamationlVT (bioreclamationivt.com/disease-state-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are pancreatic cancer tumor(s), tumor fragment(s), cells or immortalized cells. Based on the types of cells, pancreatic cancer can be categorized into exocrine tumors and pancreatic neuroendocrine tumors (PNETs). Exocrine pancreatic tumors further comprise adenocarcinoma, acinar cell carcinoma, intraductal papillary-mucinous neoplasm (ipmn), and mucinous cystadenocarcinoma (see the web at www.pancan.org/facing-pancreatic-cancer/learn/types-of-pancreatic-cancer/exocrine/). Genomic analyses of pancreatic cancer reveal a complex mutational landscape with four common oncogenic events in well-known cancer genes: KRAS, TP53, SMAD4, and CDKN2A. In addition, based on the differential expression of transcription factors and downstream targets important in lineage specification and differentiation during pancreas development and regeneration, pancreatic cancers are classified into four subtypes: (1) squamous; (2) pancreatic progenitor; (3) immunogenic; and (4) aberrantly differentiated endocrine exocrine (ADEX). Genetic markers for squamous tumors include gene networks involved in inflammation, hypoxia response, metabolic reprogramming, TGF-β signaling, MYC pathway activation, autophagy and upregulated expression of TP63AN and its target genes. Exemplary genes include TP53, KDM6A, MLL2, MLL3, PDX1, MNX1, GATA6, and HNF1B. The pancreatic progenitor subtype is characterized by transcriptional networks containing transcription factors PDX1, MNX1, HNF4G, HNF4A, HNF1B, HNF1A, FOXA2, FOXA3, and HES1. Genetic markers associated with ADEX include transcription factors NR5A2, MIST1 (also known as BHLHA15A), and RBPJL and their downstream targets; genes associated with endocrine differentiation and MODY (including INS, NEUROD1, NKX2-2, and MAFA), and genes associated with terminally differentiated pancreatic tumors, including AMY2B, PRSS1, PRSS3, CEL, and INS. The immunogenic subtype shares many of the characteristics of the pancreatic progenitor subtype, but is associated with evidence of a significant immune infiltrate. The subtypes of pancreatic cancer, genetic markers associated with each subtype, and methods for detecting are described in Bailey P, et al. (2016). Genomic analyses identify molecular subtypes of pancreatic cancer. Nature 531(7592):47-52.

In one embodiment, the pancreatic cancer tumor(s), tumor fragment(s), tumor cells or immortalized cells are obtained from a human patient. Pancreatic cell lines with known genetic information can be obtained from commercial sources, such as ATCC or Sigma. For example, ATCC® TCP-1026™ is a Pancreatic Cancer Panel including 7 pancreatic cancer cell lines with identified genetic marker. The genetic mutation information of most commonly used cancer cell lines is available from COSMIC cell line database. In addition, commonly used pancreatic cancer cell lines with KRAS, TP53, SMAD4, and CDKN2A genetic information are described in Deer E L, Gonzalez-Hernández J, Coursen J D, Shea J E, Ngatia J, Scaife C L, Firpo M A, and Mulvihill S J (2010). Phenotype and genotype of pancreatic cancer cell lines. Pancreas. 2010 39(4):425-35. Pancreatic cancer tumors are commercially available, for example, from US Biomax Inc. (biomax.us/tissue-section.php?product=HuFTU), CureLine (cureline.com/human-tumor-tissues), or BioreclamationlVT (bioreclamationivt.com/disease-state-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

In one embodiment, the tumor(s), tumor fragment(s), cells or immortalized cells are testicular cancer tumor(s), tumor fragment(s), cells or immortalized cells. Testicular tumors are divided into two major subtypes: germ cell and stromal tumors. Three well-established serum biomarkers, AFP, HCG, and LDH, are used in the diagnosis, prognosis, and surveillance of testicular tumors. Other molecular markers associated with testicular tumors include HMGA1, HMGA2, OCT3/4 (a transcription factor of the family of octamer-binding proteins (also known as the POU homeodomain proteins)), SOX2, SOX17, and CDK10. In addition, genetic loci located within KITLG, TERT, SPRY4, BAK1, DMRT1, ATF7IP, HPGDS, SMARCAD1, SEPT4, TEX14, RAD51C, PPM1E, TRIM37, MAD1L1, TEX14, SKA2, SMARCAD1, RFWD3, and RAD51C, etc., have also been associated with testicular germ cell tumor. The genetic markers can be assessed using various genotyping platforms, such as the 5′ exonuclease assay (TaqMan™), the ABI prism 7900HT sequence detection system, or the iPLEX mass array platform (Sequenom, Inc.). The genetic markers and methods of detection are described in Kreydin E I, Barrisford G W, Feldman A S, and Preston M A (2013). Testicular cancer: what the radiologist needs to know. AJR Am J Roentgenol. 200(6):1215-25; Leman E S and Gonzalgo M L (2010). Prognostic features and markers for testicular cancer management. Indian J Urol. 26(1):76-81; and Chung C C (2013). Meta-analysis identifies four new loci associated with testicular germ cell tumor. Nat Genet. 45(6):680-5.

In one embodiment, the testicular cancer tumor(s), tumor fragment(s), tumor cells or immortalized cells are obtained from a human patient. Testicular tumor cell lines are commercially available, for example, from ATCC or Sigma (e.g., NTERA-2 clone DD. The genetic mutation information of the cell line is available from COSMIC cell line database. In addition, the cell line 833K-E, derived from a human testicular germ cell tumor, has been described in Bronson D L, Andrews P W, Solter D, Cervenka J, Lange P H, and Fraley E E (1980). Cell line derived from a metastasis of a human testicular germ cell tumor. Cancer Res. 40(7):2500-6. More information on the 833K-E cell line is available at ExPAsy (Accession No. 2292). Testicular tumor tumors are commercially available, for example, from US Biomax Inc. (biomax.us/tissue-section.php?product=HuFTU) or BioreclamationIVT (bioreclamationivt.com/disease-state-tissues). In addition, a comprehensive directory of biobanks, tissue banks, and biorepositories can be accessed at specimencentral.com/biobank-directory. Further, fresh frozen tumors, including custom tissue collection, are commercially available, for example, from ProteoGenex, Inc. (proteogenex.com/biorepository/human-tissue-specimens/fresh-frozen-tissues/).

Many shapes and sizes of tumor or tumor fragment(s) are suitable. By way of example, in various embodiments, suitable shapes of tumor(s) or tumor fragment include, planar, rectangular, cuboidal, triangular, pyramidal, pentagonal, hexagonal, cylindrical, spheroidal, ovoid, and irregular. By way of example, in various embodiments, suitable sizes of tumor or tumor fragment(s) include about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 μm, or more, including increments therein. By way of further example, in various embodiments, suitable sizes of tumor or tumor fragment(s) include about 1, 2, 3, 4, 5 mm, or more, including increments therein. In light of the disclosure provided herein, those of skill the art will recognize that a minimum size is, in some embodiments, determined, at least in part, by the ability of the tumor or tumor fragment(s) to be handled and manipulated, by a human or a machine, to facilitate engraftment. Also, in light of the disclosure provided herein, those of skill the art will recognize that a maximum size is, in some embodiments, determined, at least in part, by the ability of nutrients and gasses to reach the center of the tumor or tumor fragment(s).

In some embodiments, the methods of fabricating three-dimensional, engineered, biological cancer models comprise a second maturation step. Specifically, maturing the three dimensional, engineered, biological cancer tumor model in a cell culture media. In further embodiments, the second maturation of the three-dimensional, engineered, biological cancer tumor model in a cell culture media allows the opening to close.

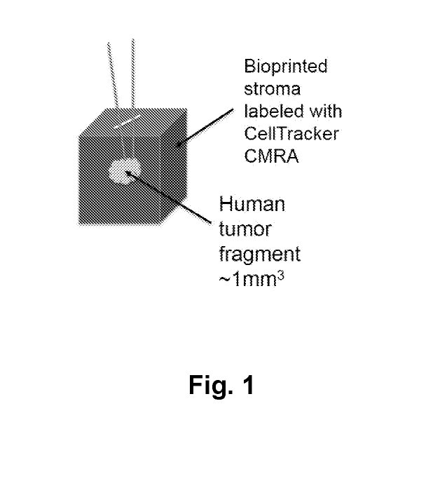

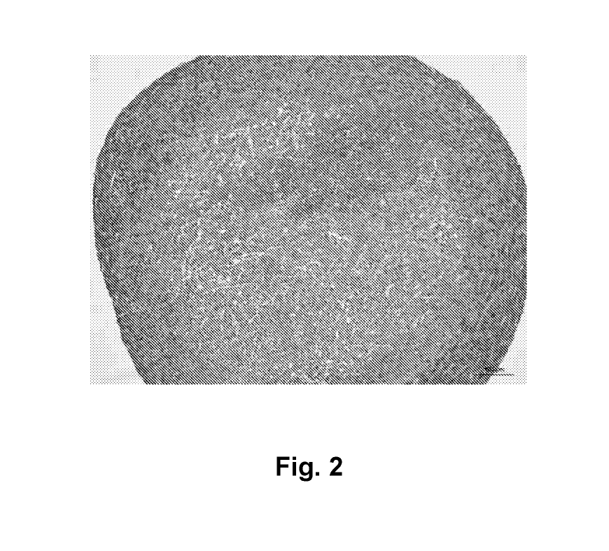

Referring to FIG. 1, in a particular embodiment, bioprinted human breast stromal tissues composed of normal mammary fibroblasts, HUVECs, and preadipocytes are labeled with a red-fluorescent cell marker and cultured in a rolling bioreactor. In this embodiment, an incision is made in the surface (white line) and a small piece of human breast cancer tissue was inserted with a pair of forceps. Further, in this embodiment, the wound closes itself, and the composite tissue is returned to culture in the rolling bioreactor. See FIG. 2 (H&E stain of a tissue conditioned in a rolling bioreactor for 7 days).

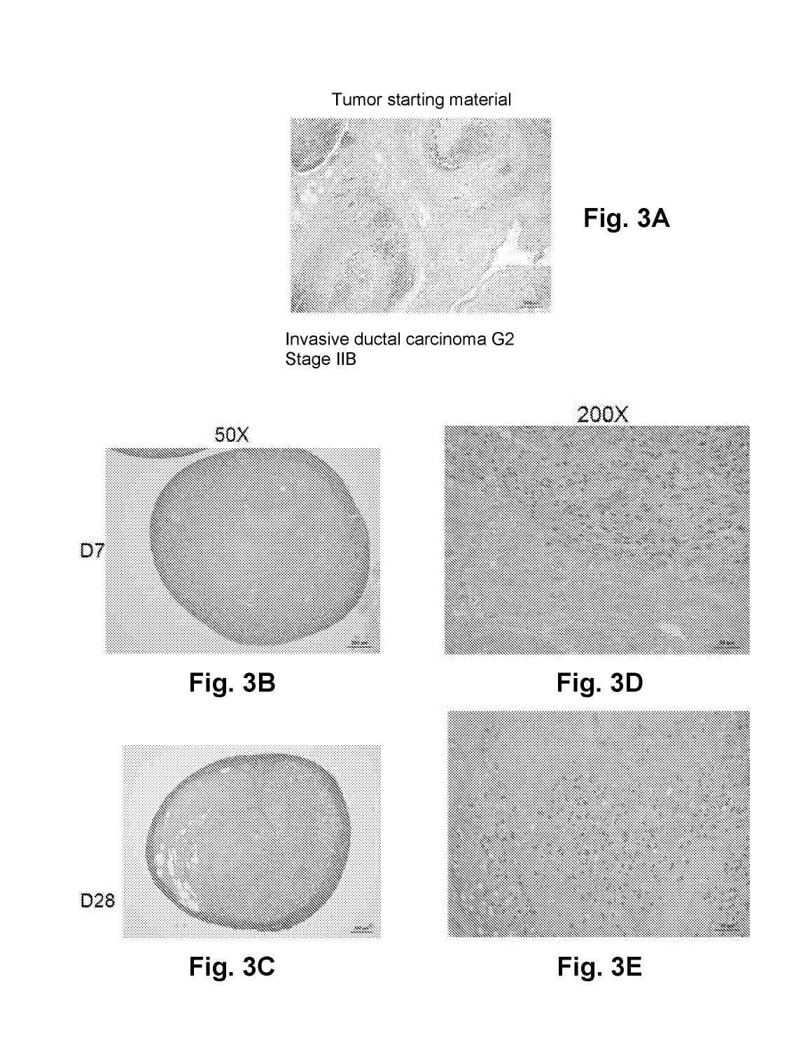

Referring to FIG. 3A-3E, in a particular embodiment, primary tumor starting material exhibits high levels of immune infiltrate and low levels of carcinoma cells (FIG. 3A, H&E staining). In this embodiment, small pieces of tumor are excised from the larger mass and implanted into bioprinted stromal tissues and perfused for 7 days or 28 days (FIGS. 3B-3E). Further, in this embodiment, representative H&E images at 50× and 200× indicate that the tumor pieces engraft into the bioprinted stroma and the stroma begins to infiltrate and remodel (arrows).

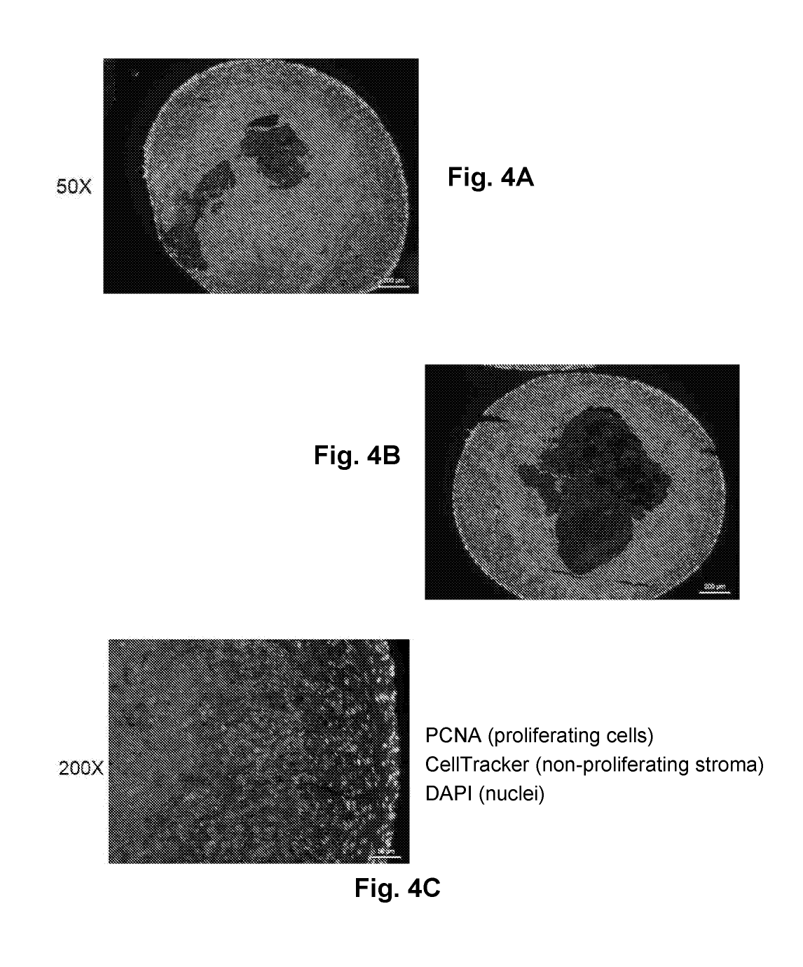

Referring to FIG. 4A-4C, in a particular embodiment, bioprinted stroma implanted with primary tumor are stained for PCNA (green in color figures) to assess proliferation, with two representative tissues shown (FIG. 4B). In this embodiment, higher magnification (FIG. 4C) indicates that the outermost stroma cells are proliferating and losing expression of the red CellTracker dye. Position of the tumor implant is indicated by the absence of staining.

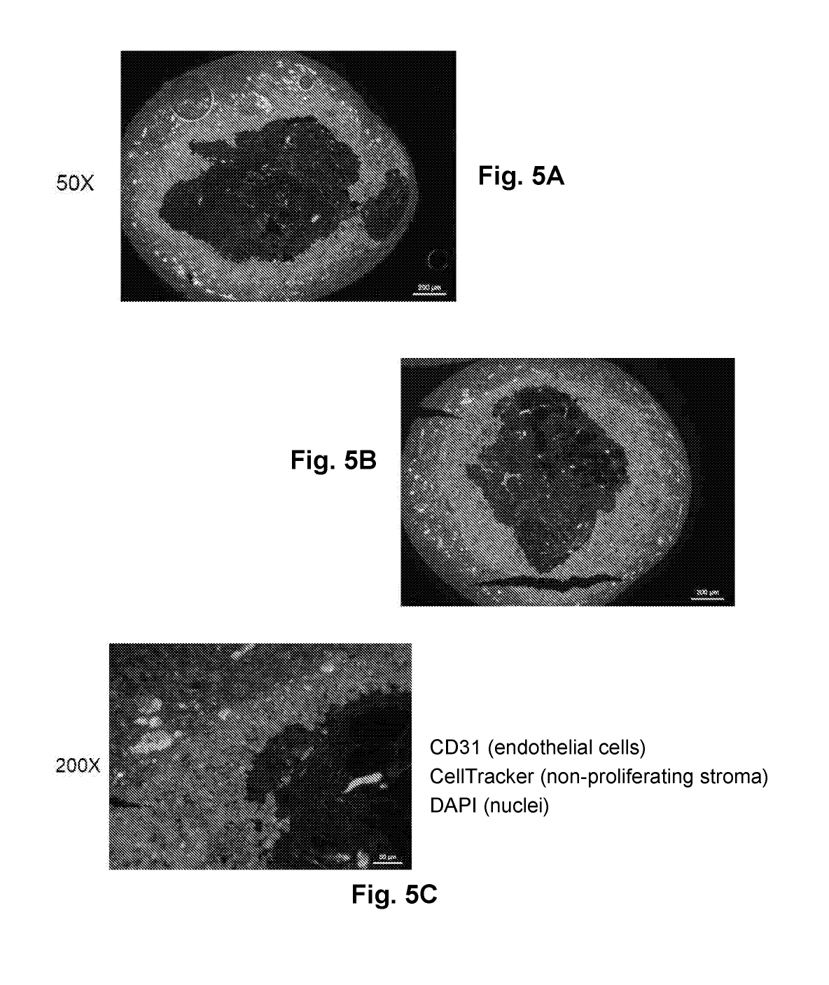

Referring to FIG. 5A-5C, in a particular embodiment, bioprinted stroma implanted with primary tumor are stained for CD31 (green in color figures) to assess organization of endothelial cell networks, with two representative tissues shown (FIGS. 5A and 5B). In this embodiment, higher magnification (FIG. 5C) indicates that bioprinted endothelial cells exhibit organization and branching in the stromal portion of the tissue, and native endothelial cells are retained in the patient tumor fragment. Position of the tumor implant is indicated by the absence of staining.

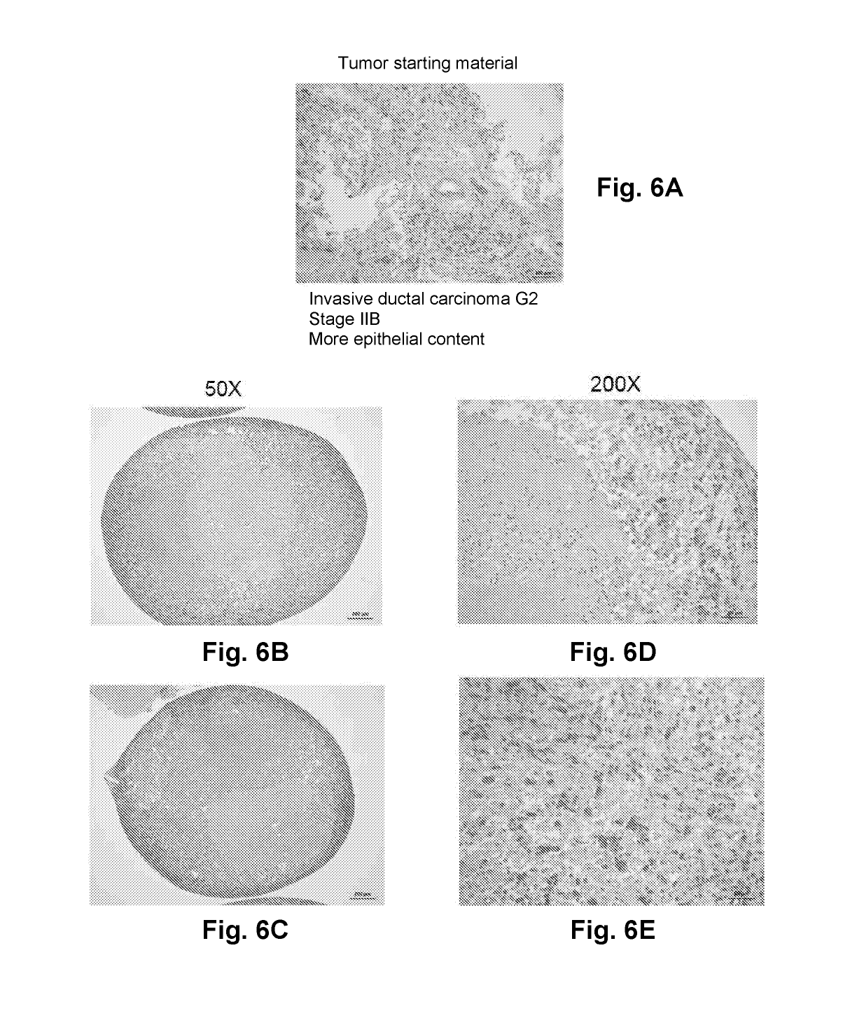

Referring to FIG. 6A-6E, in a particular embodiment, primary tumor starting material, exhibits a large number of carcinoma cells (FIG. 6A, H&E staining). In this embodiment, small pieces of tumor are excised from the larger mass and implanted into bioprinted stromal tissues and perfused for 7 days (FIGS. 6B-6E). Further, in this embodiment, representative H&E images at 50× and 200× from two tissues indicate increased retention of patient tumor cells and infiltration into the bioprinted stroma.

In some embodiments, the methods comprise identifying or evaluating a therapeutic agent for treating a disease condition in an individual (e.g., for use in personalized medicine). In further embodiments, the therapeutic agent is a cancer therapy. Accordingly, in some embodiments, the methods of identifying a therapeutic agent for cancer in an individual comprise applying a candidate therapeutic agent to the construct. In other embodiments, the methods comprise identifying a therapeutic agent in drug discovery for treatment of cancer.

In some embodiments, the methods of identifying a candidate therapeutic agent for cancer in an individual further comprise measuring an effect on the cancer cells, tumor or tumor fragment(s). In further embodiments, the effect includes apoptosis, damage to, or reduced viability of, the cancer cells, tumor, tumor fragment(s), or immortalized cells. In further embodiments, damage to, or reduced viability of, the cancer cells is assessed by way of one or more biomarkers, functional effects, or structural effects. In other embodiments, the effect includes the measurement of the size of a tumor or tumor cell fragment(s) in a cancer model contacted with the therapeutic agent compared to the size of the tumor or tumor cell fragment(s) in a model that has not been contacted with the candidate therapeutic agent, or has been contacted with a different candidate or known cancer therapeutic agent. In some embodiments, the methods of identifying a therapeutic agent for cancer in an individual comprise selecting a therapeutic agent for the individual based on the measured effect of the therapeutic agent on the cancer cells, tumor, tumor fragment(s), tumor cells, or immortalized cells.

### Further Exemplary Uses

Further described herein is the utilization of the three-dimensional, engineered, biological cancer model to measure the proliferation and engraftment potential of patient-derived tumor, tumor fragment(s) or tumor cells for the purpose of evaluating the anti-cancer therapies and developing better targeted imaging tools. The three-dimensional, engineered, biological cancer model may also be used as an ex vivo platform for developing targeted MM agents. For example, a panel of contrast agents may be coupled to estrogen, progesterone, testosterone, etc., which will bind to their respective receptors on cancer cells to identify the cancer type without the need for a biopsy.

Performing MM on small animals is challenging. The three-dimensional, engineered, biological cancer model is useful because it mimics, to variable degrees under the control of the tissue engineer, the complexity of a native tissue (without fully replicating said complexity) and may be used in the development of imaging reagents prior to use in humans.

### Animal Models

Also provided is a non-human animal model of cancer comprising a non-human animal implanted therein the three-dimensional, engineered, biological cancer model described herein. In one embodiment, the non-human animal is selected from the group consisting of any species including but not limited to murine, ovine, canine, bovine, porcine and any non-human primates. In a particular embodiment, the non-human animal is a rodent. In another particular embodiment, the non-human animal is an immunodeficient rodent. In a more specific embodiment, the animal is a NOD SCID gamma mouse. The cancer model may be implanted in any part of the non-human animal. In one embodiment, the cancer tissue model is implanted in the peritoneum of the non-human animal. In another embodiment, the cancer tissue model is subcutaneously implanted into the non-human animal. In another embodiment, the cancer tissue model is subcutaneously implanted into the flank of a rodent.

In one embodiment, a non-human animal model of cancer comprises a three-dimensional, engineered, biological cancer model comprising a three-dimensional, engineered tissue construct comprising a stromal tissue and a tumor tissue, wherein the tumor tissue is inside the stromal tissue, and the stromal tissue was bioprinted from a stromal bio-ink; and a non-human animal comprising the three-dimensional, engineered, biological cancer model, provided that the cancer model is implanted into the non-human animal after the tumor tissue is cohered to the stromal tissue. In one embodiment, the non-human animal is a genetically engineered rodent. In one embodiment, the non-human animal is an immunodeficient rodent. In one embodiment, the three-dimensional, engineered, biological cancer model does not comprise a mature perfusable vascular network, does not comprise mature red blood cells, does not comprise innervation, does not comprise neural tissue, or combinations thereof. In one embodiment, the tumor tissue comprises a plurality of undissociated, primary tumor, primary tumor fragments, primary tumor cells or immortalized cells.

In one embodiment, the tumor tissue comprises a tumor tissue selected from the group consisting of intestinal, lung, gastric, prostate, kidney, skin, ovarian, cervical, uterine, liver, bladder, esophageal, pancreatic and testicular tumor tissue. In one embodiment, the stromal tissue comprises breast stromal cells, lung stromal cells, liver stromal cells, kidney stromal cells, prostate stromal cells, intestinal stromal cells, pancreatic stromal cells or skin stromal cells. In one embodiment, the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231. In one embodiment, the tumor tissue is a pancreatic tumor tissue, and the pancreatic tumor tissue comprises markers from a pancreatic cell line, such as OPTR3099C, CAPAN1, CAPAN2, PANC1, MIAPACA2, CFPAC1, ASPC1, COL0357, PANC89, or HPAFII. In one embodiment, the tumor tissue is surrounded on all sides by the stromal tissue. In one embodiment, the cancer model is substantially free of pre-formed scaffold. In one embodiment, the tumor tissue was bioprinted. In one embodiment, the cancer model is about 1 to about 3 mm on each side. In one embodiment, the cancer model is about 0.25 to about 1 mm on each side.

In another embodiment, the non-human animal model of cancer further comprises at least one type of immune cells. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof. In one embodiment, the immune cells are lymphocytes. In one embodiment, the lymphocytes are selected from the group consisting of natural killer (NK) cells, T cells, B cells and combinations thereof. In one embodiment, the three-dimensional, engineered, biological cancer model was subcutaneously implanted into the non-human animal. In one embodiment, the non-human animal comprises the three-dimensional, engineered, biological cancer model as a subcutaneous implant. In one embodiment, the stromal tissue comprises connective tissue cells derived from a mesoderm.

Also provided is a method of making a non-human animal model of cancer comprising depositing a stromal bio-ink by bioprinting, wherein the stromal bio-ink comprises a stromal tissue, depositing a tumor tissue inside the stromal tissue, maturing the deposited stromal tissue and the deposited tumor tissue in a cell culture media to allow the stromal tissue to cohere to the tumor tissue to form a three-dimensional, engineered, biological cancer model, and implanting the cohered three-dimensional, engineered, biological cancer model into a non-human animal. In one embodiment, the non-human animal is a genetically engineered rodent. In one embodiment, the non-human animal is an immunodeficient rodent. In one embodiment, the three-dimensional, engineered, biological cancer model does not comprise a mature perfusable vascular network, does not comprise mature red blood cells, does not comprise innervation, does not comprise neural tissue, or combinations thereof. In one embodiment, the tumor tissue comprises a plurality of undissociated, primary tumor, primary tumor fragments, primary tumor cells or immortalized cells. In one embodiment, the tumor tissue comprises a tumor tissue selected from the group consisting of intestinal, lung, gastric, prostate, kidney, skin, ovarian, cervical, uterine, liver, bladder, esophageal, pancreatic and testicular tumor tissue. In one embodiment, the stromal tissue comprises breast stromal cells, lung stromal cells, liver stromal cells, kidney stromal cells, prostate stromal cells, intestinal stromal cells, pancreatic stromal cells or skin stromal cells. In one embodiment, the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231. In one embodiment, the tumor tissue is a pancreatic tumor tissue, and the pancreatic tumor tissue comprises markers from a pancreatic cell line, such as OPTR3099C, CAPAN1, CAPAN2, PANC1, MIAPACA2, CFPAC1, ASPC1, COL0357, PANC89, or HPAFII. In one embodiment, the tumor tissue is surrounded on all sides by the stromal tissue. In one embodiment, the cancer model is substantially free of pre-formed scaffold. In one embodiment, the tumor tissue was bioprinted. In one embodiment, the cancer model is about 1 to about 3 mm on each side. In one embodiment, the cancer model is about 0.25 to about 1 mm on each side.

In one embodiment, the method further comprises the step of depositing the tumor tissue by bioprinting. In one embodiment, the method further comprises the step of depositing at least one type of immune cells. In one embodiment, the method further comprises the step of bioprinting the immune cells by extrusion. In one embodiment, the immune cells are myeloid-lineage cells. In one embodiment, the myeloid-lineage cells are selected from the group consisting of monocytes, macrophages, pre-differentiated macrophages, neutrophils, basophils, eosinophils, dendritic cells, megakaryocytes and combinations thereof. In one embodiment, the immune cells are lymphocytes. In one embodiment, the lymphocytes are selected from the group consisting of natural killer (NK) cells, T cells, B cells and combinations thereof.

In one embodiment, the method further comprises the step of implanting the cancer model into the non-human animal is by subcutaneous implantation. In one embodiment, the method further comprises the step of implanting the cancer model into a flank of the non-human animal.

Also provided is a method of identifying a therapeutic agent for cancer comprising depositing a stromal bio-ink by bioprinting, the stromal bio-ink comprising a stromal tissue, depositing a tumor tissue inside the stromal tissue, wherein the tumor tissue comprises a plurality of cancer cells, maturing the deposited stromal tissue and the deposited tumor tissue in a cell culture media to allow the stromal tissue to cohere to the tumor tissue to form a three-dimensional, engineered, biological cancer model, implanting the cohered three-dimensional, engineered, biological cancer model into a non-human animal, applying a candidate therapeutic agent to the cancer model, measuring viability of the cancer cells, and selecting a therapeutic agent based on the measured viability of the cancer cells. In one embodiment, the method further comprises applying the candidate therapeutic agent to the implanted cancer model. In one embodiment, the method further comprises the step of removing the implanted cancer model from the non-human animal, wherein the candidate therapeutic agent is applied to the cancer model after the cancer model is removed from the non-human animal.

In one embodiment, provided is a method of a candidate therapeutic agent for treatment of cancer, comprising:

(a) contacting a non-human animal model described herein with the candidate therapeutic agent;

(b) measuring an effect on the primary tumor, primary tumor fragment(s), primary tumor cells or immortalized cells and/or the connective tissue cells derived from the mesoderm; and

(c) selecting the therapeutic agent for treatment of cancer based upon the measured effect.

In one embodiment, the effect on the primary tumor, primary tumor fragment(s), primary tumor cells or immortalized cells is measured by one or more of

(d) detecting any reduction of the size of the primary tumor or primary tumor fragment(s);

(e) detecting any reduction in the growth of the primary tumor, primary tumor fragment(s), primary tumor cells or immortalized cells;

detecting apoptosis in the primary tumor, primary tumor fragment(s), primary tumor cells or immortalized cells;

(g) detecting the extent of damage of the primary tumor, primary tumor fragment(s), primary tumor cells or immortalized cells;

(h) detecting reduced viability of the primary tumor, primary tumor fragment(s), primary tumor cells or immortalized cells;

(i) detecting the appearance, level or disappearance of cell markers on the primary tumor, primary tumor fragment(s), primary tumor cells or immortalized cells;

(j) detecting a change in the rate of proliferation or growth of the primary tumor, primary tumor fragment(s), primary tumor cells or immortalized cells;

(k) detecting a change in the staining of the primary tumor, primary tumor fragment(s), primary tumor cells or immortalized cells;

(l) detecting a change in RNA or DNA and/or expression thereof in the primary tumor, primary tumor fragment(s), primary tumor cells or immortalized cells;

(m) detecting a change in protein expression in the primary tumor, primary tumor fragment(s), primary tumor cells or immortalized cells;

(n) detecting a change in cytokine expression and/or secretion and/or level in the primary tumor, primary tumor fragment(s), primary tumor cells or immortalized cells; or

(o) detecting T-cell recruitment, myeloid-lineage cell recruitment, infiltration and/or activation in the primary tumor, primary tumor fragment(s), primary tumor cells or immortalized cells.

The method of contacting is by administering to the non-human animal by any method, including oral administration, by injection, or by inhalation.

### EXAMPLES

The following illustrative examples are representative of embodiments of the methods and tumor models described herein and are not meant to be limiting in any way.

### Example 1—Bioprinting of Stromal Tissue Block

The stromal compartment comprised human mammary fibroblasts, endothelial cells, and preadipocytes printed as a solid cube measuring approximately 2 mm×2 mm×2 mm. In order to track the position of the bioprinted cells or implanted cells, a red fluorescent dye (CellTracker CMRA Orange (ThermoFisher)) was incorporated into the bioprinted tissue.

### Example 2—Preconditioning of Stromal Tissue Block

Bioprinted stromal tissues were cross-linked with 50 mM calcium chloride and cultured in a rolling bioreactor in 50 mL vented cap tubes (CellTreat) at 18 rpm for 3 days. Tissues were treated with 50 mg/mL alginate lyase overnight in the bioreactor tube, and cultured in the bioreactor for 4 additional days in culture media to prepare the stromal tissues. Preconditioned tissues cultured in the rolling bioreactor exhibited a dense capsule of fibroblasts on the outer surface of the tissue that permitted incision with a scalpel. See FIG. 2.

### Example 3—Implantation of Primary Human Solid Tumor Fragment into Stromal Tissue Block

Small segments of human breast cancer tissue (approximately 0.5 mm×0.5 mm×0.5 mm) were then implanted into the interior of the bioprinted stromal tissue following the creation of a small incision. The wound induced during implantation is self-sealing and does not require a suture or adhesive to retain the tumor tissue in the interior of the structure.

### Example 4—Measurement of Engraftment of Implanted Tumor Cells into the Human Stromal Capsule

Following implantation, tumor tissues were cultured for at least 7 days. Engraftment of the tumor cell into the stromal capsule was characterized by histology. See FIGS. 3A-3E. Representative H&E images at 50× and 200× indicate that the tumor pieces engraft into the bioprinted stroma.

### Example 5—Measurement of Cellular Remodeling of Implanted Tumor Cells in the Human Stromal Capsule

Representative H&E images show the patient-derived tumor cell material integrating into the bioprinted stromal tissue within 7 days or 28 days (FIGS. 3A-3E). The tumor material continued to undergo remodeling at 28 days of culture as evidenced by adipocytes moving toward the outer part of the tissue and infiltration of the bioprinted stroma into the patient-derived material. Little or no epithelial or cellular material was detected within the starting breast tumor material (implanted, patient-derived), which may be reflective of the small amount of epithelial cells found in the individual patient's tumor.

### Example 6—Measurement of Increased Tumor Cell Heterogeneity from Tumor Cells with a Higher Carcinoma Epithelial Cell Content Remodeled in the Human Stromal Capsule

The effect of the number of carcinoma epithelial cells in the tumor starting material on infiltration of the tumor cells into the stroma was tested. Using donor tumor cell material containing a higher number of carcinoma cells, an increased infiltration and retention of implanted tumor cells into the stroma leading to a high degree of heterogeneity was observed. See FIGS. 6A-6E. The increase in heterogeneity allows the study of how different parts of the same tumor respond to anti-cancer compounds.

### Example 7—Measurement of Proliferation of Implanted Tumor Cells

Proliferation was assessed by staining the bioprinted stroma implanted with primary tumor with PCNA (green in color figures) to assess proliferation of the stroma or tumor tissue. FIGS. 4A-4C shows proliferation in the stromal compartment was observed (FIG. 4B, 50× (stroma) and 50× (tumor)). Higher magnification (FIG. 4C, 200× (stroma)) indicates that the outermost stroma cells are proliferating and losing expression of the red CellTracker Red CMTPX dye (ThermoFisher Scientific). This demonstrates that the outermost cells are most highly proliferative. The position of the tumor implant is identified by the absence of red staining. Endothelial cell networks were observed in the implanted tissue as well as in the bioprinted stromal tissue following culture for 7 days. See FIGS. 5A-5C.

### Example 8—Flow Perfusion Provides a Better Model

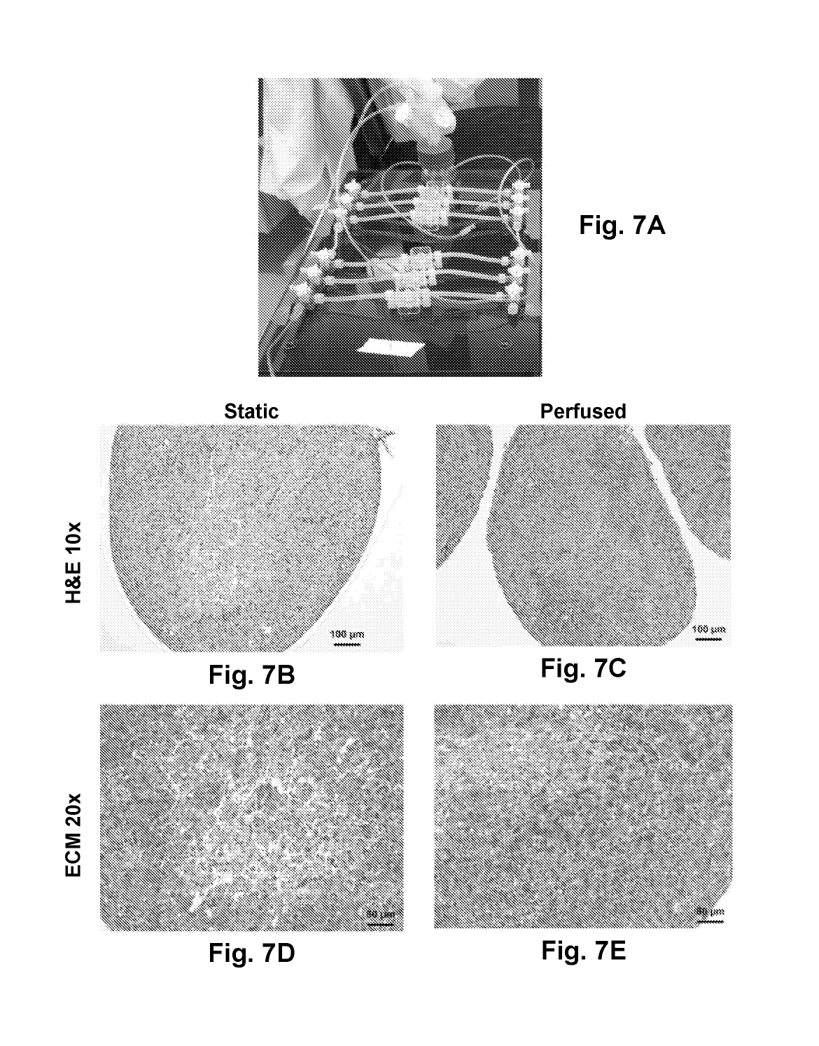

FIGS. 7A-7E depict lateral flow of media across a bioprinted breast 3D cancer model comprising MCF7 breast cancer cells surrounded by stroma comprising mammary fibroblasts, endothelial cells, and adipocytes. FIG. 7A is a photograph of 6 bioreactors that are perfused in parallel with cell culture medial to provide lateral flow. Lateral recirculating perfusion permits continuous feeding or dosing with compounds or drugs. And, lateral flow better mimics the in vivo microenvironment. FIGS. 7B-7E are micrographs showing that flow conditions enhance ECM organization and tissue cohesion.

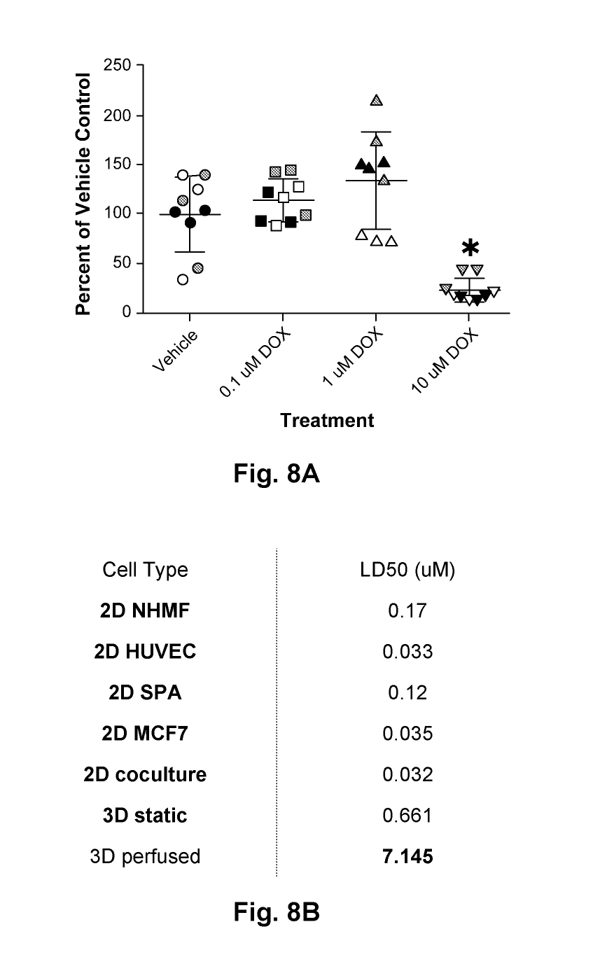

FIGS. 8A-8B show that 3D bioprinted breast cancer models subject to flow perfusion exhibit increased resistance to doxorubicin, thus providing a more accurate model compared to 2D co-cultured cells and static 3D bioprinted cells. FIG. 8A is a graph showing doxorubicin toxicity to 3D breast cancer models for vehicle (control) and increasing concentrations of doxorubicin. FIG. 8B is a graph showing the doxorubicin LD₅₀ (μM) of cultured 2D normal human mammary fibroblasts (NHMF), 2D human umbilical vein endothelial cells (HUVEC), 2D subcutaneous pre-adipocytes (SPA), 2D MCF7 cancer cells, 2D co-cultured cells (all cell types, mixed), 3D breast cancer models with static culture and 3D breast cancer models with flow perfusion.

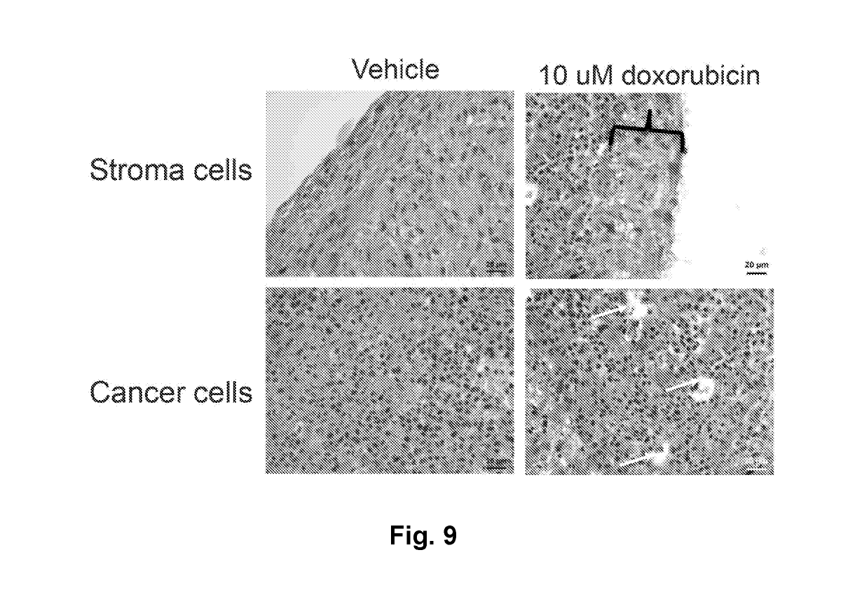

FIG. 9 depicts micrographs showing that cell-type specific effects can be observed in 3D breast cancer models subject to flow perfusion. The upper left hand panel is a micrograph showing stromal cells contacted with vehicle with flow perfusion. The upper right hand panel is a micrograph showing the effect of 10 μM doxorubicin on stromal cells subject to flow perfusion. The lower left hand panel is a micrograph showing cancer cells contacted with vehicle with flow perfusion. The lower right hand panel is a micrograph showing the effect of 10 μM doxorubicin on cancer cells subject to flow perfusion.

### Example 9—Xenografted, 3D Bioprinted Breast Tissue Containing Cancer Cells

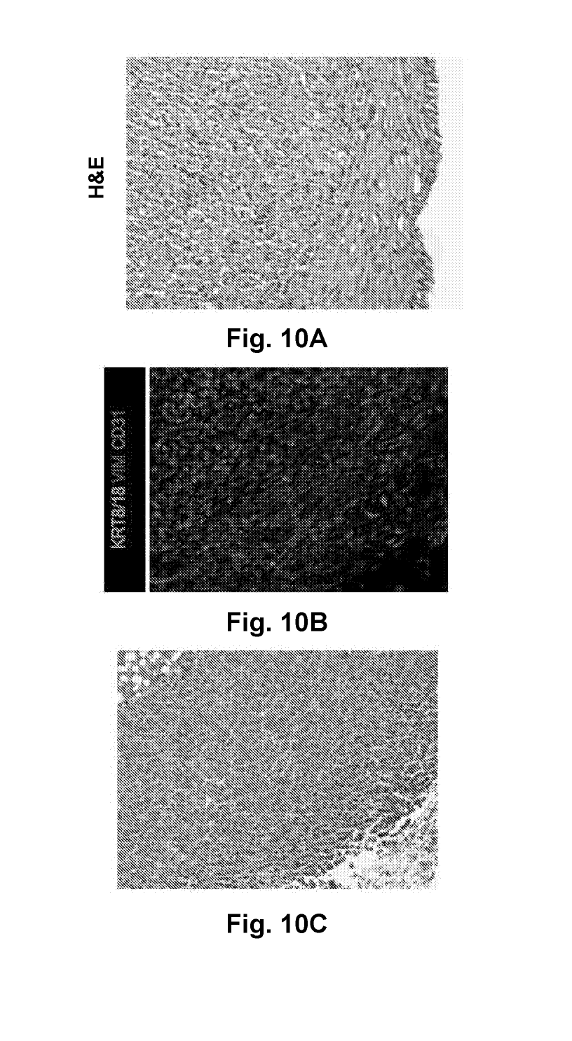

FIG. 10A shows the H&E staining of a 3D bioprinted breast cancer tissue model containing MDA-MB-231 breast cancer cells. FIG. 10B shows the immunofluorescence of the 3D bioprinted breast tissue containing MDA-MB-231 breast cancer cells, with staining for KRT8/18, Vimentin, and CD31. FIG. 10C shows the H&E staining of a xenograft derived from the 3D bioprinted breast tissue containing MDA-MB-231 breast cancer cells. FIGS. 10A-C collectively show that the MDA-MB-231 breast cancer cells printed into the 3D bioprinted breast tissue retained their tumorigenic properties and grew as xenografts.

The 3D bioprinted breast cancer tissue model of FIG. 10 was bioprinted using the Novogen Bioprinter® Instrument (Organovo, Inc., San Diego, Calif.) onto 0.4 um Transwell clear polyester membrane inserts (Corning Costar, Corning, N.Y.). The cells for each compartment, stromal or cancer, were combined and resuspended in Novogel 3.0 (Organovo, Inc., San Diego, Calif.) to a final concentration of 1.5-2.0×10⁸ cells/mL. The stromal tissue compartment comprised human mammary fibroblasts (HMF) and human umbilical vein endothelial cells (HUVEC). The tumor tissue compartment comprised breast cancer cells from the claudin low MDA-MB-231 cell line and HUVECs. The bioprinted tissue measured approximately 2 mm×2 mm×1 mm. Following bioprinting, tissues were cultured in media comprising supplements used to support each of the cell types included. Two days after printing, tissues were treated with lyase (Sigma-Aldrich) to remove the Novogel. Tissues were maintained in culture for 10 days, with media exchanges every day. At day 10, 3D bioprinted breast cancer tissue was removed from the tissue culture transwell, coated in Matrigel, and implanted subcutaneously into the flank of an immunocompromised mouse. Tumors were calipered over time and harvested at 1.5 cm in diameter. Tumor tissue was then formalin fixed and paraffin embedded for subsequent histological analysis. Importantly, as shown in FIG. 10A-C, the implanted cancer cells retained their tumorigenic properties and grew as xenografts.

### Example 10—Xenografted, 3D Bioprinted Pancreatic Tissue Containing Cancer Cells

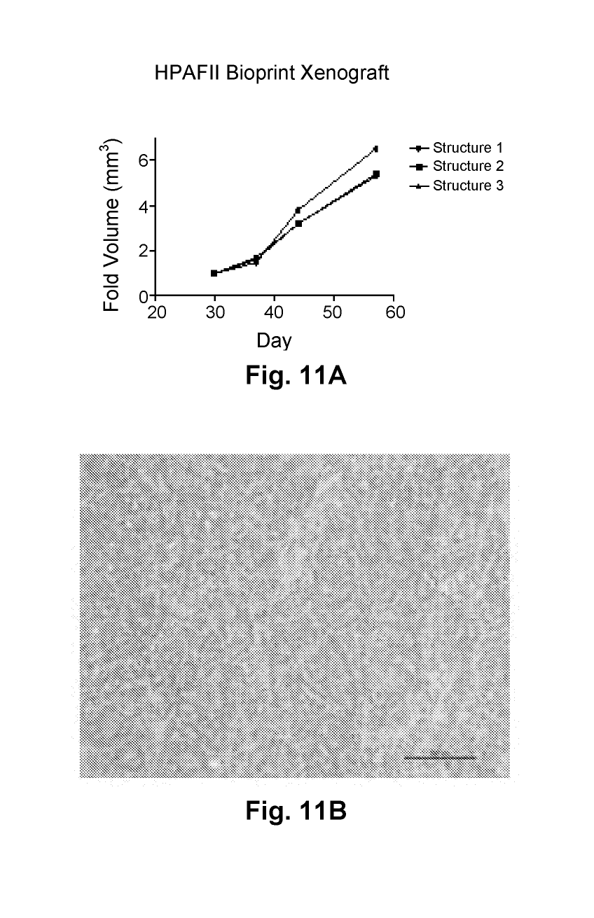

FIG. 11A is a graph that shows the growth of a 3D, bioprinted pancreatic tissue subcutaneously xenografted into three individual NSG immunodeficient mice over time. In particular, the growth was determined as the fold volume increase measured in mm³, and the graph shows that there was at least a 5× fold volume increase over a time period of 30 days. Following tumor engraftment, the mice could be used to determine the efficacy of individual therapeutic drugs, or could be used to serially expand the pancreatic tumor samples to provide larger amounts of patient tumor material for in vitro or in vivo drug screening.

FIG. 11B is the H&E staining of pancreatic tumor tissue generated from xenografted, 3D, bioprinted pancreatic tissue containing pancreatic cancer cells, with the scale bar representing 100 μm. The histology depicted in FIG. 11B shows that the implantation of the 3D, bioprinted pancreatic tissue containing pancreatic cancer cells into NSG immunodeficient mice led to robust tumor formation in vivo. The 3D bioprinted pancreatic cancer tissue model of FIG. 11 was bioprinted using the Novogen Bioprinter™ Instrument (Organovo, Inc., San Diego, Calif.) onto 0.4 um Transwell clear polyester membrane inserts (Corning Costar, Corning, N.Y.). The cells for each compartment, stromal or cancer, were combined and resuspended in Novogel 3.0 (Organovo, Inc., San Diego, Calif.) to a final concentration of 1.5-2.0×108 cells/mL. The stromal tissue compartment comprised human umbilical vein endothelial cells (HUVEC) and human primary pancreatic stellate cells (PSC). PSCs are fibroblast-like cells that are thought to be responsible for the dense desmoplastic reaction found in patients having pancreatic cancer, such as pancreatic adenocarcinoma (PDA). The tumor tissue compartment comprised HPAFII pancreatic cancer cells. Alternatively, the 3D bioprinted pancreatic cancer tissue model can be made using pancreatic epithelial cells that have been engineered to have different states of transformation that led to pancreatic cancer, such as HPNE, HPNE+E6/E7, HPNE+E6/E7+KRAS, HPNE+E6/E7+KRAS+Small T.

The bioprinted tissue measured approximately 2 mm×2 mm×1 mm. Following bioprinting, tissues were cultured in media comprising supplements used to support each of the cell types included. Tissues were maintained in culture for 10 days, with media exchanges every day. At day 10, this 3D bioprinted pancreatic cancer tissue model was subcutaneously implanted into the flank of an immunocompromised mouse. Tumors were calipered over time and harvested at 1.5 cm in diameter. Tumor tissue was then formalin fixed and paraffin embedded for subsequent histological analysis. Importantly, as shown in FIG. 11A-B, the implanted cancer cells retained their tumorigenic properties, led to robust tumor formation in vivo, and grew as xenografts.

### Example 11—Three-Dimensional, Engineered, Bioprinted Human Breast Cancer Tissue Construct Comprising Immune Cells

Breast cancer frequently shows large populations of infiltrating macrophages. A high density of infiltrating macrophages is associated with high tumor grade and worsened prognosis for both relapse-free and overall survival of breast cancer patients. For instance, high numbers of tumor associated macrophages (TAM) are positively correlated with worsened prognosis of patients with a triple negative breast cancer (TNBC), ER−/PR−/HER2− breast cancer.

There are two main types of macrophages, M1-like macrophages and M2-like macrophages. M1-like macrophages, also called classically activated macrophages, which tend to be pro-inflammatory, anti-tumorigenic and initiate adaptive immune system. By contrast, M2-like macrophages, also called alternatively-activated macrophages, tend to be anti-inflammatory (i.e. wound healing), immune suppressive, and pro-tumorigenic. Most tumor associated macrophages (TAMs) are M2-like macrophages. Thus, co-culture of triple negative breast cancer cells (TNBC: ER−/PR−/HER2− breast cancer) and monocytes may differentiate the monocytes into M2-like macrophages which in turn support the growth of TNBC cells.

A three-dimensional (3D), engineered, bioprinted human breast cancer tissue construct comprising immune cells was produced using the Novogen Bioprinter® Instrument (Organovo, Inc., San Diego, Calif.). The immune cells were myeloid-lineage cells and more particularly, monocytes from human peripheral blood mononuclear cell (PBMC)-derived myeloid lineage cells.

Stromal tissue was bioprinted from a stromal bio-ink comprising a mixture of human mammary fibroblasts (HMF), human umbilical vein endothelial cells (HUVEC), monocytes, and adipocytes. Thus, the bioprinted stromal tissue comprised a mixture of HMF, HUVEC, monocytes, and adipocytes. In particular, the monocytes were from human peripheral blood mononuclear cell (PBMC)-derived myeloid lineage cells. The tumor tissue was bioprinted from a tumor bioink comprised of a mixture of fibroblasts, HUVEC, monocytes, and breast cancer cells from the MDA-MB 231 cell line. The MDA-MB 231 cell line is a triple negative breast cancer line (TNBC: ER−/PR−/HER2). Thus, the bioprinted tumor tissue comprised of a mixture of fibroblasts, HUVEC, monocytes, and ER−/PR−/HER2− breast cancer cells. Alternatively, as noted throughout this disclosure, other breast cancer cell lines can be used in lieu of the MDA-MB 231 cell line. In this example, the bioprinted tumor tissue was located inside the bioprinted stromal tissue, such that the bioprinted tumor tissue was completely surrounded on all sides by the bioprinted stromal tissue.

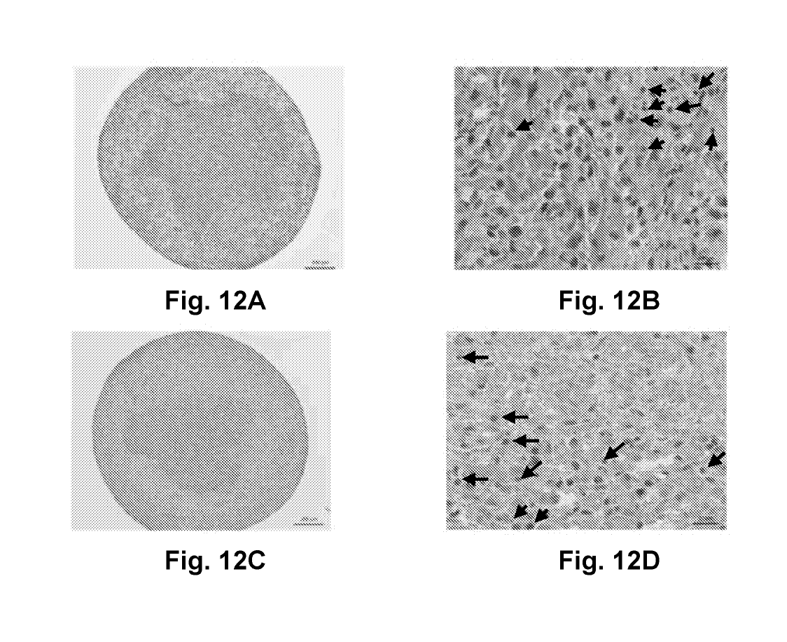

After bio-printing, the human breast cancer tissue construct was cultured in 6 well plates with cell culture media for up to 14 days post bio-printing. FIGS. 12A-B show the H&E staining of this breast cancer tissue construct comprising immune cells at day 7 post bio-printing. FIGS. 12C-D show the H&E staining of this breast cancer tissue construct comprising immune cells at day 14 post bio-printing. FIGS. 12A and 12C are H&E staining at 5× magnification, while FIGS. 12B and 12D are H&E staining at 40× magnification. FIGS. 12A and 12C show that the bioprinted tumor tissue was well capsulated by the bioprinted stromal tissue, such that the bioprinted tumor tissue was surrounded by all sides by the bioprinted stromal tissue. In FIGS. 12B and 12D, the arrows indicate macrophages that were differentiated from the monocytes. Significantly, as shown in FIG. 12D, most of the monocytes in these tissue constructs had differentiated into macrophages, and monocytes were rarely found in the tissue construct. Also significant was that most of the macrophages were found in and near the tumor tissue region, indicating the possibility of migration of differentiated macrophages toward cancer core, as shown in FIGS. 12B and D. FIG. 12D also show increased levels of steatosis.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A three-dimensional, engineered tissue construct consisting of connective tissue cells derived from the mesoderm and exhibiting a capsule of fibroblasts or fibroblast-like cells on the outer surface of the tissue construct.

2. The three-dimensional, engineered tissue construct of claim 1, which does not comprise a mature perfusable vascular network, does not comprise mature red blood cells, does not comprise innervation, does not comprise neural tissue, or combinations thereof.

3. The three-dimensional, engineered tissue construct of claim 1, wherein the connective tissue cells are stromal cells.

4. The three-dimensional, engineered tissue construct of claim 3, wherein said stromal cells are breast stromal cells, lung stromal cells, liver stromal cells, kidney stromal cells, prostate stromal cells, intestinal stromal cells, pancreatic stromal cells or skin stromal cells.

5. The three-dimensional, engineered tissue construct of claim any one of claims 1-4, comprising fibroblasts or fibroblast-like cells and at least one other stromal cell type selected from the group consisting of endothelial cells, adipocytes, pre-adipocytes, myoblasts, pericytes, osteocytes, chondrocytes and stellates.

6. The three-dimensional, engineered tissue construct of any one of claims 3-5, wherein the stromal cells are human mammary fibroblasts, human endothelial cells, human adipocytes, preadipocytes or a mixture of human adipocytes and human preadipocytes.

7. The three-dimensional, engineered tissue construct of any one of claims 1-6, which is 1 to 3 mm on each side.

8. The three-dimensional, engineered tissue construct of any one of claims 1-6, which is 0.25 to 1 mm on each side.

9. The three-dimensional, engineered tissue construct of any one of claims 1-8, wherein the capsule of fibroblasts provides a firmness that permits penetration of the construct and deposition of a cellular material within the construct while maintaining the outer form of the construct.

10. The three-dimensional, engineered tissue construct of any one of claims 1-8, wherein the capsule of fibroblasts provides a firmness that permits incision of the construct and deposition of a cellular material within the construct while maintaining the outer form of the construct.

11. The three-dimensional, engineered tissue construct of any one of claims 1-8, wherein the capsule of fibroblasts provide a firmness that permits penetration of the construct with a needle and deposition of a cellular material within the construct while maintaining the outer form of the construct.

12. The three-dimensional, engineered tissue construct of any one of claims 1-11, further comprising at least one type of immune cells.

13. The three-dimensional, engineered tissue construct of claim 12, wherein the immune cells are myeloid-lineage cells.

14. The three-dimensional, engineered tissue construct of claim 13, wherein the myeloid-lineage cells are selected from the group consisting of monocytes, macrophages, pre-differentiated macrophages, neutrophils, basophils, eosinophils, dendritic cells, megakaryocytes and combinations thereof.

15. The three-dimensional, engineered tissue construct of claim 12, wherein the immune cells are lymphocytes.

16. The three-dimensional, engineered tissue construct of claim 15, wherein the lymphocytes are selected from the group consisting of natural killer (NK) cells, T cells, B cells and combinations thereof.

17. A method of making the three-dimensional, engineered tissue construct of any one of claims 1-16, comprising
(a) preparing a bio-ink comprising the connective tissue cells derived from the mesoderm;
(b) depositing the bio-ink on a biocompatible surface to form an array of cells;
(c) maturing the deposited array of cells in a cell culture media under non-static conditions thereby producing the three-dimensional, engineered, tissue construct with fibroblasts on the outer surface of the construct.

18. The method of claim 17, wherein the bio-ink is deposited by bioprinting.

19. The method of claim 17 or 18, wherein the bio-ink comprises 55%-75% fibroblasts, 15%-35% endothelial cells, and 0%-20% adipocytes, preadipocytes, or a mixture thereof.

20. The method of any one of claims 17-19, wherein the deposited array of cells is matured in the cell culture medium for 4 to 10 days.

21. The method of any one of claims 17-20, wherein the non-static conditions apply shear stress to the deposited array of cells.

22. The method of any one of claims 17-21, wherein the non-static conditions are created by maturing the deposited array of cells in a rolling bioreactor.

23. The method of any one of claims 17-22, wherein the bio-ink further comprises at least one type of immune cells.

24. The method of claim 23, wherein the immune cells are myeloid-lineage cells.

25. The method of claim 24, wherein the myeloid-lineage cells are selected from the group consisting of monocytes, macrophages, pre-differentiated macrophages, neutrophils, basophils, eosinophils, dendritic cells, megakaryocytes, and combinations thereof.

26. The method of claim 23, wherein the immune cells are lymphocytes.

27. The method of claim 26, wherein the lymphocytes are selected from the group consisting of natural killer (NK) cells, T cells, B cells and combinations thereof.

28. A three-dimensional, engineered, biological cancer model comprising
(a) a three-dimensional, engineered tissue construct comprising connective tissue cells derived from the mesoderm and exhibiting a capsule of fibroblasts or fibroblast-like cells on the outer surface of the tissue construct, and
(b) an undissociated, primary tumor, primary tumor fragment(s), primary tumor cells or immortalized cells inside the three-dimensional, engineered tissue construct of (a).

29. The three-dimensional, engineered, biological cancer model of claim 28, wherein the
(a) three-dimensional, engineered tissue construct consists of connective tissue cells derived from the mesoderm and exhibiting a capsule of fibroblasts or fibroblast-like cells on the outer surface of the tissue construct

30. A three-dimensional, engineered, biological cancer model comprising
(a) a three-dimensional, engineered tissue construct comprising connective tissue cells derived from the mesoderm and optionally exhibiting a capsule of fibroblasts or fibroblast-like cells on the outer surface of the tissue construct, and
(b) an undissociated intestinal, lung, gastric, prostate, kidney, skin, ovarian, cervical, uterine, liver, bladder, esophageal, pancreatic or testicular primary tumor(s), primary tumor fragment(s), primary tumor cells or immortalized cells inside the three-dimensional, engineered tissue construct of (a).

31. The three-dimensional, engineered, biological cancer model of any one of claims 28-30, wherein the three-dimensional, engineered tissue construct does not comprise a mature perfusable vascular network, does not comprise mature red blood cells, does not comprise innervation, does not comprise neural tissue, or combinations thereof.

32. The three-dimensional, engineered biological cancer model of claim 28, wherein the tumor, tumor fragment(s), tumor cells or immortalize cells are breast, lung, liver, kidney, prostate, intestinal, pancreatic or skin tumors, tumor fragment(s), tumor cells or immortalized cells.

33. The three-dimensional, engineered biological cancer model of any one of claims 28-32, wherein the connective tissue cells are stromal cells.

34. The three-dimensional, engineered biological cancer model of claim 33, wherein the stromal cells are fibroblasts, endothelial cells, adipocytes, preadipocytes, a mixture of adipocytes and preadipocytes, myoblasts, pericytes, osteocytes, chondrocytes and stellates.

35. The three-dimensional, engineered biological cancer model of claim 33, wherein the stromal cells are human mammary fibroblasts, human endothelial cells, human adipocytes, human preadipocytes, or a mixture of human adipocytes and human preadipocytes.

36. The three-dimensional, engineered biological cancer model of any one of claims 28-35, which is 1 to 3 mm on each side.

37. The three-dimensional, engineered biological cancer model of any one of claims 28-35, which is 0.25 to 1 mm on each side.

38. The three-dimensional, engineered biological cancer model of any one of claims 28-37, wherein a plurality of the cancer models are in the wells of a multi-well plate.

39. The three-dimensional, engineered biological cancer model of any one of claims 28-38, further comprising at least one type of immune cells.

40. The three-dimensional, engineered biological cancer model of claim 39, wherein the immune cells are myeloid-lineage cells.

41. The three-dimensional, engineered biological cancer model of claim 40, wherein the myeloid-lineage cells are selected from the group consisting of monocytes, macrophages, pre-differentiated macrophages, neutrophils, basophils, eosinophils, dendritic cells, megakaryocytes and combinations thereof.

42. The three-dimensional, engineered biological cancer model of claim 40, wherein the immune cells are lymphocytes.

43. The three-dimensional, engineered biological cancer model of claim 42, wherein the lymphocytes are selected from the group consisting of natural killer (NK) cells, T cells, B cells and combinations thereof.

44. The three-dimensional, engineered biological cancer model of any one of claims 28-43, comprising a plurality of (i) undissociated, primary tumors, primary tumor fragments, primary tumor cells or immortalized cells or (ii) a plurality of undissociated intestinal, lung, gastric, prostate, kidney, skin, ovarian, cervical, uterine, liver, bladder, esophageal, pancreatic or testicular primary tumors, primary tumor fragments, primary tumor cells or immortalized cells within the three dimensional, engineered tissue construct comprising connective tissue from the mesoderm.

45. The three-dimensional, engineered biological cancer model of claim 44, wherein the plurality of (i) or (ii) are present in separate compartments within the three-dimensional, engineered tissue construct of (a).

46. The three-dimensional, engineered biological cancer model of claim 44 or 45, wherein each of the plurality of (i) undissociated, primary tumors, primary tumor fragments, primary tumor cells or immortalized cells or (ii) a plurality of undissociated intestinal, lung, gastric, prostate, kidney, skin, ovarian, cervical, uterine, liver, bladder, esophageal, pancreatic or testicular primary tumors, primary tumor fragments, primary tumor cells or immortalized cells represents a subtype of one or more types of cancer.

47. The three-dimensional, engineered biological cancer model of any one of claims 28-46, disposed on a solid support.

48. The three-dimensional, engineered, biological cancer model of claim 47, wherein model is disposed on a biocompatible membrane that is disposed on the solid support.

49. The three-dimensional, engineered, biological cancer model of claim 47 or 48, wherein the solid support is a multi-well plate.

50. A non-human animal model of cancer comprising a non-human animal implanted therein the three-dimensional, engineered, biological cancer model of any one of claims 28-46.

51. The non-human animal model of claim 50, wherein the non-human animal is an immunodeficient rodent.

52. A plurality of the three-dimensional, engineered, biological cancer models of any one of claims 28-46 in the form of an array.

53. The plurality of the three-dimensional, engineered, biological cancer models of claim 52, wherein the array is disposed on a solid support.

54. The plurality of the three-dimensional, engineered, biological cancer models of claim 53, wherein the array is disposed on a biocompatible membrane that is disposed on a solid support.

55. The plurality of the three-dimensional, engineered, biological cancer models of claim 53 or 54, wherein the solid support is a multi-well plate.

56. The plurality of the three-dimensional, engineered, biological cancer models of any one of claims 52-55, wherein each cancer model represents a subtype of one or more types of cancer.

57. The plurality of the three-dimensional, engineered, biological cancer models of claim 56, wherein the tumor(s), tumor fragment(s), tumor cells or immortalized cells are breast cancer tumor(s), tumor fragment(s), tumor cells or immortalized cells.

58. The plurality of the three-dimensional, engineered, biological cancer models of 57, wherein the array comprises at least two breast cancer models selected from the group consisting of breast cancer subtypes luminal A, luminal B, HER2-enriched (HER2E), basal-like, and normal breast-like.

59. The plurality of the three-dimensional, engineered, biological cancer models of 57, wherein the array comprises at least two breast cancer models expressing markers selected from the group consisting of ER+, ER−, PR+, PR−, HER2+, HER2−, and ER−/PR−/HER2−.

60. The plurality of the three-dimensional, engineered, biological cancer models of claim 56, wherein the tumor(s), tumor fragment(s), tumor cells or immortalized cells are intestinal cancer tumor(s), tumor fragment(s), tumor cells or immortalized cells.

61. The plurality of the three-dimensional, engineered, biological cancer models of 60, wherein the array comprises at least two colorectal cancer models selected from the group consisting of colorectal subtypes CMS1, CMS2, CMS3, and CMS4.

62. The plurality of the three-dimensional, engineered, biological cancer models of 60, wherein the array comprises at least two colorectal models expressing markers selected from the group consisting of MLH1, MLH2, MSH3, MSH6, PMS2, POLE and POLD1.

63. The plurality of the three-dimensional, engineered, biological cancer models of claim 56, wherein the tumor(s), tumor fragment(s), tumor cells or immortalized cells are lung cancer tumor(s), tumor fragment(s), tumor cells or immortalized cells.

64. The plurality of the three-dimensional, engineered, biological cancer models of 63, wherein the array comprises at least two lung cancer models selected from the group consisting of lung cancer subtypes squamous cell carcinoma, adenocarcinoma, large cell carcinoma, small cell lung carcinoma, and lung carcinoid tumor.

65. The plurality of the three-dimensional, engineered, biological cancer models of 63, wherein the array comprises at least two lung cancer models expressing markers selected from the group consisting of iNTR, TUBB3, RRM1, ECC1, BRCA1, p53, BCL-2, ALK, MRP2, MSH2, TS, mucin, BAG-1, pERK1/2, pAkt-1, p2′7, PARP-1, ATM and TopIIA.

66. The plurality of the three-dimensional, engineered, biological cancer models of claim 56, wherein the tumor(s), tumor fragment(s), tumor cells or immortalized cells are gastric cancer tumor(s), tumor fragment(s), tumor cells or immortalized cells.

67. The plurality of the three-dimensional, engineered, biological cancer models of 66, wherein the array comprises at least two gastric cancer models selected from the group consisting of gastric cancer subtypes mesenchymal-like type, microsatellite-unstable type, tumor protein 53 (TP53)-active type and TP53-inactive type.

68. The plurality of the three-dimensional, engineered, biological cancer models of 66, wherein the array comprises at least two gastric cancer models expressing markers selected from the group consisting of the micro RNAs miR-1, miR-20a, miR-27a, miR-34, miR-196a, miR-378, miR-221, miR376c, miR-423-5p, let-7a, miR-17-5p, miR-21, miR-106a/b, miR-199a-3p, miR-218, miR-223, miR-370, miR-451, miR-486, miR-21, miR-106a, miR-129, and miR-421; TP53; the PTKs TIE-1 and MKK4; FYN; PLK1; GISP/RegIV; EGFR; ERBB2; VEGF; TGF; c-MET; IL-6; IL-11; Cyclin E; Bc1-2; Fas; surviving; Runx3; E-cadherin; WNT5A; IL-1; IL-10; carcinoembryonic antigen (CEA); alpha-fetoprotein (AFP); CA 19-9; CA 72-4; free beta-subunit of human choriogonadotropin (B-HCG), and pepsinogen I/II.

69. The plurality of the three-dimensional, engineered, biological cancer models of claim 56, wherein the tumor(s), tumor fragment(s), tumor cells or immortalized cells are prostate cancer tumor(s), tumor fragment(s), tumor cells or immortalized cells.

70. The plurality of the three-dimensional, engineered, biological cancer models of 69, wherein the array comprises at least two prostate cancer models selected from the group consisting of prostate cancer subtypes expressing gene fusions ERG, ETV1, ETV4 and FLI1 or selected from the group consisting of prostate cancer subtypes expressing mutations SPOP, FOXA1 and IDH1.

71. The plurality of the three-dimensional, engineered, biological cancer models of 69, wherein the array comprises at least two prostate cancer models expressing markers selected from the group consisting of NKX3.1, MYC, TMPRSS2-ERG translocations, PTEN, Akt/mTOR, Erk (p42/44), Her2/Neu or SRC tyrosine kinases, WNT, APC, k-RAS, β-catenin, FGFR1, FGF10, EZH2, PCA3, and AR.

72. The plurality of the three-dimensional, engineered, biological cancer models of claim 56, wherein the tumor(s), tumor fragment(s), tumor cells or immortalized cells are kidney cancer tumor(s), tumor fragment(s), tumor cells or immortalized cells.

73. The plurality of the three-dimensional, engineered, biological cancer models of 72, wherein the array comprises at least two kidney cancer models selected from the group consisting of kidney cancer subtypes renal cell carcinoma and transitional cell carcinoma.

74. The plurality of the three-dimensional, engineered, biological cancer models of 72, wherein the renal cell carcinoma is selected from the group consisting of clear cell (conventional) (RCC), papillary RCC, chromophobe RCC, renal oncocytoma RCC, unclassified RCC, collecting duct carcinoma, medullary RCC and carcomatoid DCC.

75. The plurality of the three-dimensional, engineered, biological cancer models of 72, wherein the array comprises at least two kidney cancer models expressing markers selected from the group consisting of neuron-specific enolase (NSE), TRAF-1, Hsp27, IL-1, IL-6, TNF-α, serum amyloid A (SAA), C-reactive protein (CRP), gamma-glutamyl transferase (GGT), tumor necrosis factor-related apoptosis-inducing ligand (TRAIL), cytokeratins (CK), serum M65 (the intact form of cytokeratin 18), hypoxia-inducible transcriptional factors (HIF-1α and HIF-1β), VEGF, Von Hippel-Lindau (VHL), prolyl hydroxylase-3 (PHD3), pyruvate kinase isoenzyme type M2 (TuM2-PK), thymidine kinase 1 (TK1), 20S proteasome, Fetuin A, Osteopontin (OPN), Osteoprotegerin, NMP-22, NGAL, KIM-1, MMPs, and PLIN2.

76. The plurality of the three-dimensional, engineered, biological cancer models of claim 56, wherein the tumor(s), tumor fragment(s), tumor cells or immortalized cells are skin cancer tumor(s), tumor fragment(s), tumor cells or immortalized cells.

77. The plurality of the three-dimensional, engineered, biological cancer models of 76, wherein the array comprises at least two skin cancer models selected from the group consisting of skin cancer subtypes actinic keratosis, basal cell carcinoma, melanoma, Karposi sarcoma, merkel cell carcinoma, and squamous cell carcinoma.

78. The plurality of the three-dimensional, engineered, biological cancer models of 77, wherein the melanoma is selected from the group consisting of mutant BRAF, mutant RAS, mutant NF1, and triple-wild type.

79. The plurality of the three-dimensional, engineered, biological cancer models of 76, wherein the array comprises at least two skin cancer models expressing markers selected from the group consisting of mutant BRAF, mutant RAS, mutant NF1, Triple-WT (wild type), BRAF, NRAS, CDKN2A/B, TP53, PTEN, RAC1, MAP2K1, PPP6C, ARID2, F1, IDH1, RB1, DDX3X, RAC1, IDHL MRPS31, RPS27, TERT, phospho-MAP2K1/MAP2K2 (MEK1/2), MAPK1/MAPK3 (ERK1/2), CDK4, and CCND1.

80. The plurality of the three-dimensional, engineered, biological cancer models of claim 56, wherein the tumor(s), tumor fragment(s), tumor cells or immortalized cells are ovarian cancer tumor(s), tumor fragment(s), tumor cells or immortalized cells.

81. The plurality of the three-dimensional, engineered, biological cancer models of 80, wherein the array comprises at least two ovarian cancer models selected from the group consisting of ovarian subtypes serous, endometrioid, clear cell and mucinous.

82. The plurality of the three-dimensional, engineered, biological cancer models of 80, wherein the array comprises at least two ovarian cancer models expressing markers selected from the group consisting of B-RAF, K-RAS, TP53, BRCA1/2, CA125, CA 19.9, CA 15.3, TAG.72, MSH2, MLH1, MLH6, PMS1, PMS2, ESR2, BRIP1, MSH6, RAD51C, RAD51D, CDH1, CHEK2, PALB2, RAD50, OVX1, sFas, CYFRA 21.1, VEGF, human kallikrein 10 (hK10), Alpha-fetoprotein (αFP), M-CSF, and LDH, inhibin α, betaA, and betaB subunits.

83. The plurality of the three-dimensional, engineered, biological cancer models of claim 56, wherein the tumor(s), tumor fragment(s), tumor cells or immortalized cells are cervical cancer tumor(s), tumor fragment(s), tumor cells or immortalized cells.

84. The plurality of the three-dimensional, engineered, biological cancer models of 83, wherein the array comprises at least two cervical cancer models selected from the group consisting of cervical cancer subtypes squamous cell carcinoma and adenocarcinoma.

85. The plurality of the three-dimensional, engineered, biological cancer models of 83, wherein the array comprises at least two cervical cancer models expressing markers selected from the group consisting of p16ink4a, MCM 3 and 5, CDC6, Geminin, Cyclins A-D, TOPO2A, CDCA1, BIRC5, UBE2C, CCNB1, CCNB2, PLOD2, NUP210, MELK, CDC20, IL8, INDO, ISG15, ISG20, AGRN, DTXL, MMP1, MMP3, CCL18, STAT1, ribosomal protein S12, the mitochondrial subunit NADH dehydrogenase 4, 16S ribosomal RNA (rRNA), and capping protein muscle Z-line al.

86. The plurality of the three-dimensional, engineered, biological cancer models of claim 56, wherein the tumor(s), tumor fragment(s), tumor cells or immortalized cells are uterine cancer tumor(s), tumor fragment(s), tumor cells or immortalized cells.

87. The plurality of the three-dimensional, engineered, biological cancer models of 86, wherein the array comprises at least two uterine cancer models selected from the group consisting of uterine cancer subtypes endometrioid, adenocarcinoma, serous adenocarcinoma, adenosquamous carcinoma and carcinomasarcoma.

88. The plurality of the three-dimensional, engineered, biological cancer models of 86, wherein the array comprises at least two uterine cancer models expressing markers selected from the group consisting of MLH1, MSH2, MSH6, PMS2, EPCAM, PTEN, BRCA1, BRCA2, TP53, MUTYH, CDKN2A, PGR, and CHEK2.

89. The plurality of the three-dimensional, engineered, biological cancer models of claim 56, wherein the tumor(s), tumor fragment(s), tumor cells or immortalized cells are liver cancer tumor(s), tumor fragment(s), tumor cells or immortalized cells.

90. The plurality of the three-dimensional, engineered, biological cancer models of 89, wherein the array comprises at least two liver cancer models selected from the group consisting of liver cancer subtypes hepatocellular carcinoma (HCC), cholangiocarcinoma, angiosarcoma, and hepatoblastoma.

91. The plurality of the three-dimensional, engineered, biological cancer models of 89, wherein the array comprises at least two liver cancer models expressing markers selected from the group consisting of AFP-L1, AFP-L2, AFP-L3, HSP70, HSP27, Glypican-3 (GPC3), squamous cell carcinoma antigen (SCCA), Golgi protein 73 (GP73, also known as Golph2 and GOLM1), Tumor-associated glycoprotein 72 (TAG-72), Zinc-α2-glycoprotein (ZAG), Des-γ-carboxyprothrombin (DCP), γ-glutamyl transferase (GGT), α-1-fucosidase (AFU), Transforming growth factor-β1 (TGF-β1), VEGF, microRNAs such as miR-500, miR-122, miR-29, and miR-21; A-like 1 homolog (DLK1), Villin1 (Vil1), TP53, CD34, RGS5, THY1, ADAMTS1, MMP2, MMP14, keratin 17, keratin 19, and mucin 1.

92. The plurality of the three-dimensional, engineered, biological cancer models of claim 56, wherein the tumor(s), tumor fragment(s), tumor cells or immortalized cells are bladder cancer tumor(s), tumor fragment(s), tumor cells or immortalized cells.

93. The plurality of the three-dimensional, engineered, biological cancer models of 92, wherein the array comprises at least two bladder cancer models selected from the group consisting of bladder cancer subtypes urothelial carcinoma, squamous cell carcinoma, adenocarcinoma, sarcoma and small cell anaplastic cancer.

94. The plurality of the three-dimensional, engineered, biological cancer models of 92, wherein the array comprises at least two bladder cancer models expressing markers selected from the group consisting of HRAS, NRAS, KRAS2, FGFR3, ERBB2, CCND1, MDM2, E2F3, RASSF1A, FHIT, CDKN2A, PTCH, DBC1, TSC1, PTEN, RB1, TP53, SULF1, the lysosomal cysteine proteinases cathepsins B, K, and L; RGS1, RGS2, THBS1, THBS2, VEGFC, NRP2, CTSE, MMP2, CCNA2, CDC2, CDC6, TOP2A, SKALP PRKAG1, GAMT, ACOX1, ASAH1, SCD, AF1Q, AREG, DUSP6, LYAR, MAL, and RARRES

95. The plurality of the three-dimensional, engineered, biological cancer models of claim 56, wherein the tumor(s), tumor fragment(s), tumor cells or immortalized cells are esophageal cancer tumor(s), tumor fragment(s), tumor cells or immortalized cells.

96. The plurality of the three-dimensional, engineered, biological cancer models of 95, wherein the array comprises at least two esophageal cancer models selected from the group consisting of esophageal cancer subtypes squamous-cell carcinoma and adenocarcinoma.

97. The plurality of the three-dimensional, engineered, biological cancer models of 95, wherein the array comprises at least two esophageal cancer models expressing markers selected from the group consisting of SMYD3, RUNX1, CTNNA3, RBFOX1, CDKN2A/2B, CDK14, ERBB2, EGFR, RB1, GATA4/6, CCND1, MDM2, TP53, ARID1A, and SMARCA4.

98. The plurality of the three-dimensional, engineered, biological cancer models of claim 56, wherein the tumor(s), tumor fragment(s), tumor cells or immortalized cells are pancreatic cancer tumor(s), tumor fragment(s), tumor cells or immortalized cells.

99. The plurality of the three-dimensional, engineered, biological cancer models of 98, wherein the array comprises at least two pancreatic cancer models selected from the group consisting of pancreatic cancer subtypes exocrine and pancreatic neuroendocrine tumors (PNETs).

100. The plurality of the three-dimensional, engineered, biological cancer models of 98, wherein the array comprises at least two pancreatic cancer models selected from the group consisting of pancreatic cancer subtypes squamous, pancreatic progenitor, immunogenic and aberrantly differentiated endocrine exocrine (ADEX).

101. The plurality of the three-dimensional, engineered, biological cancer models of 98, wherein the array comprises at least two pancreatic cancer models expressing markers selected from the group consisting of TP53, KDM6A, MLL2, MLL3, PDX1, MNX1, GATA6, HNF1B, transcription factors PDX1, MNX1, HNF4G, HNF4A, HNF1B, HNF1A, FOXA2, FOXA3, HES1, NR5A2, MIST1 (also known as BHLHA15A), and RBPJL; INS, NEUROD1, NKX2-2, MAFA, AMY2B, PRSS1, PRSS3, CEL, and INS.

102. The plurality of the three-dimensional, engineered, biological cancer models of claim 56, wherein the tumor(s), tumor fragment(s), tumor cells or immortalized cells are testicular cancer tumor(s), tumor fragment(s), tumor cells or immortalized cells.

103. The plurality of the three-dimensional, engineered, biological cancer models of 102, wherein the array comprises at least two testicular cancer models selected from the group consisting of testicular cancer subtypes germ cell and stromal tumors.

104. The plurality of the three-dimensional, engineered, biological cancer models of 102, wherein the array comprises at least two testicular cancer models expressing markers selected from the group consisting of AFP, HCG, LDH, HMGA1, HMGA2, OCT3/4 (a transcription factor of the family of octamer-binding proteins (also known as the POU homeodomain proteins)), SOX2, SOX17, CDK10 and genetic loci located within KITLG, TERT, SPRY4, BAK1, DMRT1, ATF7IP, HPGDS, SMARCAD1, SEPT4, TEX14, RAD51C, PPM1E, TRIM37, MAD1L1, TEX14, SKA2, SMARCAD1, RFWD3, and RAD51C.

105. The plurality of the three-dimensional, engineered, biological cancer models of any one of claims 52-104, further comprising at least one type of immune cells in culture media that is in contact with and/or within the cancer models.

106. The plurality of the three-dimensional, engineered, biological cancer models of claim 105, wherein the immune cells are myeloid-lineage cells.

107. The plurality of the three-dimensional, engineered, biological cancer models of claim 106, wherein the myeloid cells are selected from the group consisting of monocytes, macrophages, pre-differentiated macrophages, neutrophils, basophils, eosinophils, dendritic cells, megakaryocytes, and combinations thereof.

108. The plurality of the three-dimensional, engineered, biological cancer models of claim 105, wherein the immune cells are lymphocytes.

109. The plurality of the three-dimensional, engineered, biological cancer models of claim 108, wherein the lymphocytes are selected from the group consisting of natural killer (NK) cells, T cells, B cells and combinations thereof.

110. The plurality of the three-dimensional, engineered, biological cancer models of any one of claims 52-109, wherein the cancer models are in culture media under non-static culture conditions.

111. The plurality of the three-dimensional, engineered, biological cancer models of claim 110, wherein non-static culture conditions is lateral flow across the cancer models.

112. The plurality of the three-dimensional, engineered, biological cancer models of any one of claims 52-109, wherein the cancer models are in culture media under static culture conditions.

113. The plurality of the three-dimensional, engineered, biological cancer models of any one of claims 52-112, for use in a high throughput assay.

114. A method of making the three-dimensional, engineered biological cancer model of any one of claims 28-49, comprising
(a) creating an opening in the three-dimensional, engineered tissue construct,
(b) inserting an undissociated, primary tumor, primary tumor fragment(s), primary tumor cells or immortalized cells into the opening, and
(c) maturing the three-dimensional, engineered biological cancer model in cell culture media to allow the opening to close.

115. A method of identifying a therapeutic agent for the treatment of cancer, comprising
(a) contacting a candidate therapeutic agent with the three-dimensional, engineered biological cancer model of any one of claims 28-49 or the plurality of the three-dimensional, engineered, biological cancer models of any one of claims 52-112;
(b) measuring an effect on the primary tumor, primary tumor fragment(s), primary tumor cells or immortalized cells and/or the connective tissue cells derived from the mesoderm; and
(c) selecting the therapeutic agent for treatment of cancer based upon the measured effect.

116. The method of claim 115, wherein the method is for identifying a therapeutic agent for treatment of cancer in an individual and the tumor, tumor fragment(s), tumor cells or immortalized cells derived are from that individual.

117. The method of claim 114 or 115, wherein said cancer is breast cancer, lung cancer, liver cancer, kidney cancer, prostate cancer, intestinal cancer, pancreatic cancer or skin cancer.

118. The method of claim 114 or 115, wherein said cancer is gastric cancer, ovarian cancer, cervical cancer, uterine cancer, bladder cancer, esophageal cancer, or testicular cancer.

119. The method of claim 114 or 115, wherein the stromal cells are breast stromal cells, and the tumor, tumor fragment(s), tumor cells or immortalized cells are derived from an individual having breast cancer.

120. The method of claim 114 or 115, wherein the stromal cells are lung stromal cells, and the tumor, tumor fragment(s), tumor cells or immortalized cells are derived from an individual having lung cancer.

121. The method of claim 114 or 115, wherein the stromal cells are liver stromal cells, and the tumor, tumor fragment(s), tumor cells or immortalized cells are derived from an individual having liver cancer.

122. The method of claim 114 or 115, wherein the stromal cells are kidney stromal cells, and the tumor, tumor fragment(s), tumor cells or immortalized cells are derived from an individual having kidney cancer.

123. The method of claim 114 or 115, wherein the stromal cells are prostate stromal cells, and the tumor, tumor fragment(s), tumor cells or immortalized cells are derived from an individual having prostate cancer.

124. The method of claim 114 or 115, wherein the stromal cells are intestinal stromal cells, and the tumor, tumor fragment(s), tumor cells or immortalized cells are derived from an individual having intestinal cancer.

125. The method of claim 114 or 115, wherein the stromal cells are pancreatic cells, and the tumor, tumor fragment(s), tumor cells or immortalized cells are derived from an individual having pancreatic cancer.

126. The method of claim 114 or 115, wherein the stromal cells are skin cells, and the tumor, tumor fragment(s), tumor cells or immortalized cells are derived from an individual having skin cancer.

127. The method of claim 114 or 115, wherein the stromal cells are gastric stromal cells, and the tumor, tumor fragment(s), tumor cells or immortalized cells are derived from an individual having gastric cancer.

128. The method of claim 114 or 115, wherein the stromal cells are ovarian stromal cells, and the tumor, tumor fragment(s), tumor cells or immortalized cells are derived from an individual having ovarian cancer.

129. The method of claim 114 or 115, wherein the stromal cells are cervical stromal cells, and the tumor, tumor fragment(s), tumor cells or immortalized cells are derived from an individual having cervical cancer.

130. The method of claim 114 or 115, wherein the stromal cells are uterine stromal cells, and the tumor, tumor fragment(s), tumor cells or immortalized cells are derived from an individual having uterine cancer.

131. The method of claim 114 or 115, wherein the stromal cells are bladder stromal cells, and the tumor, tumor fragment(s), tumor cells or immortalized cells are derived from an individual having bladder cancer.

132. The method of claim 114 or 115, wherein the stromal cells are esophageal stromal cells, and the tumor, tumor fragment(s), tumor cells or immortalized cells are derived from an individual having esophageal cancer.

133. The method of claim 114 or 115, wherein the stromal cells are testicular stromal cells, and the tumor, tumor fragment(s), tumor cells or immortalized cells are derived from an individual having testicular cancer.

134. The method of any one of claim 114 or 115, wherein each cancer model of the plurality of cancer models represent subtypes of a particular type of cancer.

135. The method of any one of claims 114-134, wherein the cancer models are in culture media under non-static culture conditions.

136. The method of claim 135, wherein said non-static conditions is lateral flow across the cancer models.

137. The method of any one of claims 114-134, wherein the cancer models are in culture media under static culture conditions.

138. The method of any one of claims 114-137 when carried out in a high throughput assay.

139. The method of any one of claims 114-138, wherein the effect on the primary tumor, primary tumor fragment(s), primary tumor cells or immortalized cells is measured by one or more of
(a) detecting any reduction of the size of the primary tumor or primary tumor fragment(s);
(b) detecting any reduction in the growth of the primary tumor, primary tumor fragment(s), primary tumor cells or immortalized cells;
(c) detecting apoptosis in the primary tumor, primary tumor fragment(s), primary tumor cells or immortalized cells;
(d) detecting the extent of damage of the primary tumor, primary tumor fragment(s), primary tumor cells or immortalized cells;
(e) detecting reduced viability of the primary tumor, primary tumor fragment(s), primary tumor cells or immortalized cells;
(f) detecting the appearance, level or disappearance of cell markers on the primary tumor, primary tumor fragment(s), primary tumor cells or immortalized cells;
(g) detecting a change in the rate of proliferation or growth of the primary tumor, primary tumor fragment(s), primary tumor cells or immortalized cells;
(h) detecting a change in the staining of the primary tumor, primary tumor fragment(s), primary tumor cells or immortalized cells;
(i) detecting a change in RNA or DNA and/or expression thereof in the primary tumor, primary tumor fragment(s), primary tumor cells or immortalized cells;
(j) detecting a change in protein expression in the primary tumor, primary tumor fragment(s), primary tumor cells or immortalized cells;
(k) detecting a change in cytokine expression and/or secretion and/or level in the primary tumor, primary tumor fragment(s), primary tumor cells or immortalized cells; or
(l) detecting T-cell recruitment, myeloid-lineage cell recruitment, infiltration and/or activation in the primary tumor, primary tumor fragment(s), primary tumor cells or immortalized cells.

140. A method of a candidate therapeutic agent for treatment of cancer, comprising:
(a) contacting the non-human animal model of any one of claims 50-51 with the candidate therapeutic agent;
(b) measuring an effect on the primary tumor, primary tumor fragment(s), primary tumor cells or immortalized cells and/or the connective tissue cells derived from the mesoderm; and
(c) selecting the therapeutic agent for treatment of cancer based upon the measured effect.

141. The method of claim 140, wherein the effect on the primary tumor, primary tumor fragment(s), primary tumor cells or immortalized cells is measured by one or more of
(a) detecting any reduction of the size of the primary tumor or primary tumor fragment(s);
(b) detecting any reduction in the growth of the primary tumor, primary tumor fragment(s), primary tumor cells or immortalized cells;
(c) detecting apoptosis in the primary tumor, primary tumor fragment(s), primary tumor cells or immortalized cells;
(d) detecting the extent of damage of the primary tumor, primary tumor fragment(s), primary tumor cells or immortalized cells;
(e) detecting reduced viability of the primary tumor, primary tumor fragment(s), primary tumor cells or immortalized cells;
(f) detecting the appearance, level or disappearance of cell markers on the primary tumor, primary tumor fragment(s), primary tumor cells or immortalized cells;
(g) detecting a change in the rate of proliferation or growth of the primary tumor, primary tumor fragment(s), primary tumor cells or immortalized cells;
(h) detecting a change in the staining of the primary tumor, primary tumor fragment(s), primary tumor cells or immortalized cells;
(i) detecting a change in RNA or DNA and/or expression thereof in the primary tumor, primary tumor fragment(s), primary tumor cells or immortalized cells;
(j) detecting a change in protein expression in the primary tumor, primary tumor fragment(s), primary tumor cells or immortalized cells;
(k) detecting a change in cytokine expression and/or secretion and/or level in the primary tumor, primary tumor fragment(s), primary tumor cells or immortalized cells; or
(l) detecting T-cell recruitment, myeloid-lineage cell recruitment, infiltration and/or activation in the primary tumor, primary tumor fragment(s), primary tumor cells or immortalized cells.

142. A non-human animal model of cancer comprising:
(a) a three-dimensional, engineered, biological cancer model comprising a three-dimensional, engineered tissue construct comprising a stromal tissue and a tumor tissue, wherein the tumor tissue is inside the stromal tissue, and the stromal tissue was bioprinted from a stromal bio-ink; and
(b) a non-human animal comprising the three-dimensional, engineered, biological cancer model, provided that the cancer model is implanted into the non-human animal after the tumor tissue is cohered to the stromal tissue.

143. The non-human animal model of claim 142, wherein the non-human animal is a genetically engineered rodent.

144. The non-human animal model of claim 142 or 143, wherein the non-human animal is an immunodeficient rodent.

145. The non-human animal model of any one of claims 142-144, wherein the three-dimensional, engineered, biological cancer model does not comprise a mature perfusable vascular network, does not comprise mature red blood cells, does not comprise innervation, does not comprise neural tissue, or combinations thereof.

146. The non-human animal model of any one of claims 142-145, wherein the tumor tissue comprises a plurality of undissociated, primary tumor, primary tumor fragments, primary tumor cells or immortalized cells.

147. The non-human animal model of any one of claims 142-146, wherein the stromal tissue comprises stromal cells selected from the group consisting of fibroblasts, endothelial cells, adipocytes, pre-adipocytes, a mixture of adipocytes and preadipocytes, myoblasts, pericytes, osteocytes, chondrocytes and stellates.

148. The non-human animal model of any one of claims 142-147, wherein the stromal tissue comprises breast stromal cells, lung stromal cells, liver stromal cells, kidney stromal cells, prostate stromal cells, intestinal stromal cells, pancreatic stromal cells or skin stromal cells.

149. The non-human animal model of any one of claims 142-148, wherein the tumor tissue comprises a tumor tissue selected from the group consisting of intestinal, lung, gastric, prostate, kidney, skin, ovarian, cervical, uterine, liver, bladder, esophageal, pancreatic and testicular tumor tissue.

150. The non-human animal model of any one of claims 142-148, wherein the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231.

151. The non-human animal model of any one of claims 142-149, wherein the tumor tissue is a pancreatic tumor tissue, and the pancreatic tumor tissue comprises markers from a pancreatic cell line.

152. The non-human animal model of claim 151, wherein the pancreatic cell line is selected from the group consisting of OPTR3099C, CAPAN1, CAPAN2, PANC1, MIAPACA2, CFPAC1, ASPC1, COL0357, PANC89, and HPAFII.

153. The non-human animal model of any one of claims 142-152, wherein the tumor tissue is surrounded on all sides by the stromal tissue.

154. The non-human animal model of any one of claims 142-153, wherein the cancer model is substantially free of pre-formed scaffold.

155. The non-human animal model of any one of claims 142-154, wherein the tumor tissue was bioprinted.

156. The non-human animal model of any one of claims 142-155, wherein the cancer model is about 1 to about 3 mm on each side.

157. The non-human animal model of any one of claims 142-155, wherein the cancer model is about 0.25 to about 1 mm on each side.

158. The non-human animal model of any one of claims 142-157, further comprising at least one type of immune cells.

159. The non-human animal model of claim 158, wherein the immune cells are myeloid-lineage cells.

160. The non-human animal model of claim 159, wherein the myeloid-lineage cells are selected from the group consisting of monocytes, macrophages, pre-differentiated macrophages, neutrophils, basophils, eosinophils, dendritic cells, megakaryocytes and combinations thereof.

161. The non-human animal model of claim 158, wherein the immune cells are lymphocytes.

162. The non-human animal model of claim 161, wherein the lymphocytes are selected from the group consisting of natural killer (NK) cells, T cells, B cells and combinations thereof.

163. The non-human animal model of any one of claims 142-162, wherein the three-dimensional, engineered, biological cancer model of (a) was subcutaneously implanted into the non-human animal.

164. The non-human animal model of any one of claims 142-163, wherein the stromal tissue comprises connective tissue cells derived from a mesoderm.

165. The non-human animal model of any one of claims 142-164, wherein the tumor tissue comprises primary cancer cells from a patient tumor.

166. A method of making a non-human animal model of cancer comprising:
depositing a stromal bio-ink by bioprinting, wherein the stromal bio-ink comprises a stromal tissue;
depositing a tumor tissue inside the stromal tissue;
maturing the deposited stromal tissue and the deposited tumor tissue in a cell culture media to allow the stromal tissue to cohere to the tumor tissue to form a three-dimensional, engineered, biological cancer model; and
implanting the cohered three-dimensional, engineered, biological cancer model into a non-human animal.

167. A method of identifying a therapeutic agent for cancer comprising:
depositing a stromal bio-ink by bioprinting, the stromal bio-ink comprising a stromal tissue;
depositing a tumor tissue inside the stromal tissue, wherein the tumor tissue comprises a plurality of cancer cells;
maturing the deposited stromal tissue and the deposited tumor tissue in a cell culture media to allow the stromal tissue to cohere to the tumor tissue to form a three-dimensional, engineered, biological cancer model;
implanting the cohered three-dimensional, engineered, biological cancer model into a non-human animal;
applying a candidate therapeutic agent to the cancer model;
measuring viability of the cancer cells; and
selecting a therapeutic agent based on the measured viability of the cancer cells.

168. The method of claim 166 or 167, wherein the non-human animal is a genetically engineered rodent.

169. The method of any one of claims 166-168, wherein the non-human animal is an immunodeficient rodent.

170. The method of any one of claims 166-169, wherein the three-dimensional, engineered, biological cancer model does not comprise a mature perfusable vascular network, does not comprise mature red blood cells, does not comprise innervation, does not comprise neural tissue, or combinations thereof.

171. The method of any one of claims 166-170, wherein the tumor tissue comprises a plurality of undissociated, primary tumor, primary tumor fragments, primary tumor cells or immortalized cells.

172. The method of any one of claims 166-171, wherein the stromal tissue comprises stromal cells selected from the group consisting of fibroblasts, endothelial cells, adipocytes, pre-adipocytes, a mixture of adipocytes and preadipocytes, myoblasts, pericytes, osteocytes, chondrocytes and stellates.

173. The method of any one of claims 166-172, wherein the stromal tissue comprises breast stromal cells, lung stromal cells, liver stromal cells, kidney stromal cells, prostate stromal cells, intestinal stromal cells, pancreatic stromal cells or skin stromal cells.

174. The method of any one of claims 166-173, wherein the tumor tissue comprises a tumor tissue selected from the group consisting of intestinal, lung, gastric, prostate, kidney, skin, ovarian, cervical, uterine, liver, bladder, esophageal, pancreatic and testicular tumor tissue.

175. The method of any one of claims 166-173, wherein the tumor tissue is a breast tumor tissue, and the breast tumor tissue comprises cell lines selected from the group consisting of ER+, ER−, PR+, PR−, HER2+, HER2−, ER−/PR−/HER2−, MCF-7, SKBR3, HCC1143, and MDA-MB-231.

176. The method of any one of claims 166-174, wherein the tumor tissue is a pancreatic tumor tissue, and the pancreatic tumor tissue comprises markers from a pancreatic cell line.

177. The method of claim 176, wherein the pancreatic cell line is selected from the group consisting of OPTR3099C, CAPAN1, CAPAN2, PANC1, MIAPACA2, CFPAC1, ASPC1, COL0357, PANC89, and HPAFII.

178. The method of any one of claims 166-177, wherein the tumor tissue is surrounded on all sides by the stromal tissue.

179. The method of any one of claims 166-178, wherein the cancer model is substantially free of pre-formed scaffold.

180. The method of any one of claims 166-179, further comprising the step of depositing the tumor tissue by bioprinting.

181. The method of any one of claims 166-180, wherein the bioprinting is by extrusion.

182. The method of any one of claims 166-181, wherein the cancer model is about 1 to about 3 mm on each side.

183. The method of any one of claims 166-181, wherein the cancer model is about 0.25 to 1 mm on each side.

184. The method of any one of claims 166-183, further comprising the step of depositing at least one type of immune cells.

185. The method of claim 184, wherein the step of depositing the at least one type of immune cells is by bioprinting.

186. The method of claim 185, wherein the bioprinting is by extrusion.

187. The method of any one of claims 184-186, wherein the immune cells are myeloid-lineage cells.

188. The method of claim 187, wherein the myeloid-lineage cells are selected from the group consisting of monocytes, macrophages, pre-differentiated macrophages, neutrophils, basophils, eosinophils, dendritic cells, megakaryocytes and combinations thereof.

189. The method of any one of claims 184-186, wherein the immune cells are lymphocytes.

190. The method of claim 189, wherein the lymphocytes are selected from the group consisting of natural killer (NK) cells, T cells, B cells and combinations thereof.

191. The method of any one of claims 166-190, wherein the step of implanting the cancer model into the non-human animal is by subcutaneous implantation.

192. The method of claim 191, wherein the tumor model is implanted into a flank of the non-human animal.

193. The method of any one of claims 166-192, wherein the stromal tissue comprises connective tissue cells derived from a mesoderm.

194. The methods of any one of claims 166-193, wherein the cancer cells are primary cancer cells from a patient tumor.

195. The method of any one of claims 166-194, wherein the candidate therapeutic agent is applied to the implanted cancer model.

196. The method of any one of claims 166-195, further comprising the step of removing the implanted cancer model from the non-human animal, wherein the candidate therapeutic agent is applied to the cancer model after the cancer model is removed from the non-human animal.

197. The method of any one of claim 115-141 or 167-196, wherein the candidate therapeutic agent is an immunotherapy.

198. The method of claim 197, wherein the immunotherapy is an adoptive T cell transfer, an immune checkpoint inhibitor to activate Tc and NK cells, or an immune cell reprogramming and depletion.

199. A three-dimensional, engineered, biological breast cancer model comprising:
(a) breast stromal tissue, the stromal tissue comprising fibroblasts, endothelial cells, adipocytes, and monocytes; and
(b) breast cancer tumor tissue; the tumor tissue comprising breast cancer cells, fibroblasts, endothelial cells, and monocytes; the tumor tissue surrounded on all sides by the stromal tissue to form the three-dimensional, engineered, biological breast cancer model;
provided that the stromal tissue was bioprinted from a stromal bio-ink, the tumor tissue was bioprinted from a tumor bio-ink, or both the stromal tissue and the tumor tissue were bioprinted from their respective bio-inks.

200. The breast cancer model of claim 199, wherein the model is substantially free of pre-formed scaffold.

201. The breast cancer model of claim 199 or 200, wherein the breast cancer cells are derived from a breast cancer cell line.

202. The breast cancer model of claim 201, wherein the breast cancer cell line is selected from the group consisting of ER+, ER−, PR+, PR−, HER2+, HER2−, and ER−/PR−/HER2−.

203. The breast cancer model of claim 199 or 200, wherein the breast cancer cells are primary cancer cells from a patient tumor.

204. The breast cancer model of any one of claims 199-203, wherein the breast cancer tumor tissue is completely surrounded on all sides by the breast stromal tissue to form the three-dimensional, engineered, biological breast cancer model.

205. The breast cancer model of any one of claims 199-204, further comprising a plurality of macrophages that were differentiated from the monocytes.

206. A method of fabricating a three-dimensional, engineered, biological breast cancer model, the method comprising:
(a) preparing a stromal bio-ink, the stromal bio-ink comprising a plurality of stromal cell types, the stromal cell types comprising: an extrusion compound, fibroblasts, endothelial cells, monocytes, and adipocytes;
(b) preparing a tumor bio-ink, the tumor bio-ink comprising: an extrusion compound, a breast cancer cell type, fibroblasts, and monocytes;
(c) depositing the stromal bio-ink and the tumor bio-ink such that the tumor bio-ink is embedded in the stromal bio-ink and in contact with the stromal bio-ink on all sides; and
(d) maturing the deposited bio-ink in a cell culture media to remove the extrusion compound to allow the cells to cohere to form a three-dimensional, engineered, biological breast cancer model.

207. The method of claim 206, wherein the bio-ink is deposited by bioprinting.

208. The method of claim 206 or 207, wherein the breast cancer cell type comprises a breast cancer cell line.

209. The method of claim 208, wherein the breast cancer cell line is selected from the group consisting of ER+, ER−, PR+, PR−, HER2+, HER2−, and ER−/PR−/HER2−.

210. The method of any one of claims 206-209, wherein the cancer cell type comprises primary breast cancer cells from a patient tumor.

211. The method of any one of claims 206-210, further comprising the step of allowing the monocytes to differentiate into a plurality of macrophages.

212. The method of claim 211, further comprising the step of allowing the macrophages to migrate towards the breast cancer cell types.

213. The method of any one of claims 206-212, further comprising the steps of:
applying a candidate therapeutic agent to the three-dimensional, engineered, biological breast cancer model;
measuring viability of the cancer cells; and
selecting a therapeutic agent for the individual based on the measured viability of the cancer cells.

214. The method of claim 213, wherein the candidate therapeutic agent is an immunotherapy.

215. The method of claim 214, wherein the immunotherapy is an adoptive T cell transfer, an immune checkpoint inhibitor to activate Tc and NK cells, or an immune cell reprogramming and depletion.
